SEQUENCE LISTING (5 sequences)

SEQ ID NO: 1 - DNA, 93358 bp, Pseudomonas protegens
  misc_feature 1..93358: note = Orfamide biosynthesis gene cluster
  source 1..93358: mol_type = genomic DNA
atgctggaaccaagtcataacgacgagctcccggtcattccgggcaagcgctacttcaccattggtgaggtcagtgagctctgtgcggtcaagccgcatgtgctgcgttattgggagcaggagtttcctcaactcaaccccgtcaagcgccgcggaaatcgccggtattatcagcgccaggatgtgctgatgatccggcagatccgcgcgttgctttatgaccaggggttcaccatcggcggggcgcgtctgcgtctttccggtgatgaggccaaagacgatacgacccagtacaagcaactgatccgtcagatgatctctgagctggaagatgtgcttgtggtgctaaaaaagtaattctctgttttaaatacttccacttttcaaaagcttgcggtatattcctcaacgtttcctgagaagtgaaacagatttcacgcctagtcggggcgtagcgcagtccggtagcgcactagcatggggtgctaggggtcgagtgttcgaatcactccgtcccgaccatatttttcaatgacttagccgctttcgagcggctttgttgtttctgnnctgccatagtgacttttcgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactttcttgcgcaactgcgaagcttctgcnnnnnnncgaagcttctccggctttttcacgctcgcgggttcggcctggcttgctaggttatcggctcgatgggggggagggcgcacgggtggcaactactcctgccacgtccatgacttggtgcttgttgcggctggctcactctgcctagtggagggtggcaatgcgccggttttcgccaatcctgattcggtccagcgctcggttgagtgcgtccagggcattgagcaaggcttgtgcctcggtttcccgaccctgaccccagaggcgctcggccatcttgttcaaggcctggatggagcgctcgatatcggcggcagtgactgccttggcatcctccggctcttttctaggcatgggctgaggcctcgattgtggtttgctggcgaactggatgatgggtttgccagtgttcgatggagggctgtttccggttcggtctggcgagattcatggaaggcctgtcactgacgacatttcaaggccgcggcttgtatgtcggaggcgctaccgacgttgacgacgctcatgctgtcggcgccaagttcggctgctttattgaaggccatattcagcgcgtccccattggaggagcccttcacggtaaatgccgtcagaaccttgcaggcctgtgccgaggaggccgtcaccagatcgacctgccttcctgcttccgtcagttgtgtggtgcaagccgagaccgaagccgcagcgacgacaagcaagacaaggccacgcagattacgcatcactgaacactcctgtcgacgaggttgccggcctatcccggccagccagccccgggcaatcatcgcgagtacatcgcccaccagaacacatggcccaggatcgagatctgctgttcctggatttcggcgaagctatagtcttcgtccggatgttcatcccgattgaagctgcgcaggcgcagcccgaccggcaaacggtaaacctgcttcacgcgcaactggccattgtggttgagtgcatacatctccccgtcgactacatccttcagggcattcctgccgacgttcacgccaacggttgcgccgtcgcgcagcaccgggaccatgctgttgccgctgaccgacacgcacttggcgttgttgaactgcaccccgttctgacgcagatctttcttgaagaagcgcagccgggcggtatcgctttcttcgatgacaaaccggcccgagcctgcagccagctccacctcgtgaagaaaggggacgtagacctcgttgtcatcgagcggggtcgcatcgtcccaggtctcgatatcgtccaaatgcacgcttggctgggtgcgtgccgatgcggtatccgcggccttgagcatttctccaacgccttcagcgagccacatggaggaaacgccgcataccgaggcaatctgggcgacaaaggccgtggccttcgatttgccacgttccaggtcggagattgaggtttgggtcaggccggcacgttcagcgagctctgtttgattgagctgggcgtggcgacgagctgttttgagtcgatctttaaattccatccgcccagtattacgggggttcccataagcttgcaaatgggtattcctatattgtactatatgggtgttcccgtaagggtgtcaggcagataatcaaatggatttgcagatgttaaaagaatgcagatgtggaagttgcaatcgacttctcgctcgagtgggcgagtttactgagctccagatcaaatgttcccgctgtggaaccttgaatcatgtaaaggccgcgagtctcgagctatcgccaggagcggccaggtgtgaaagcggcgtcggtcattgagcctgtgatgcaaaggcacgtccgggccagaggcccgaactcctgttgcatgacggttccaggccacgcgtagcgctgtcgcagtcattattggaatgggtggagctctcacgccgtggcggtctgaagctatcgcccccttcgtgaaagccaagagcttcgatttcctcgcctttgagcgacatgcaggcggaatcctccgcatccatcattcgactcaataggtaacaacatggcttggattcgatacgactttaccgacaacggttcttctgtactgccagcgcgctactacatggcgcccaaccagtaccttcagtcccctaacaagcgcttcaagctgctctttcaggcagatggcaacctggcactgtatgacggcgcccagttggcatgggtggctgaccagaacacgccgttcaccaacgtgagcaagggcaacaagaaagatccgatgatggtcttcatgaactacggcttngtcctggatgacccgctgcgtggccgtatctggtcgaccactccgagtgaccccaccatgggttcgcgcgaagatgcctcgctgcgtgcgttcacccagcttcaggacgacgggaacatcgtcactgtcgacaccatcccgcactggtatgcgcccaatggtcgtgtattcaccccggcggtgggtacggcaatgatcatcagcggtccggccgagctggtaccgggccagctctacagcgctggcgatcacgctctggtcttccagggcgatggcaacctggtggtgtacgggccgaactggagcgtggtctgggcgacctatacccagaacaaaggtggtgtgcgatgcgtcatgcaggcagacggcaacctggtgatctacgccgcgaacaatgcggtcgtctggcagagcggcacggctggtcaccccggcgcaaccatccgcctgcaggccaacggcagtttcacgatagtgaccgagcggccgatctgggcccgcttcggctataccccgaccatcaagccgccgcgtgtgttctaccccgatcacaagtggaaaaccggtagctacacctgggacaacgtgttctaacggcctgcaagaggccccgcgacgatgcggggcttctacaggcttcactcaagggcccggcaggcttgttctgccgggctctgcccattggtggcgcagattgaacggggtcacgaacgggcaagcgacagctggccttcaaaggccctgctctcaaccttgaaaaacagattctcctgagtaccgccattcacctgcttcaggacggtgtgcgacaactgcgcgccgattttctccatggctcgctgtgagcggatattcgaacccgcaacatggaaccagacggtgtcgacccactgaaaggcgtgcccgagcatcaggtacttgagttccttgttggcactgcccccccatttttcgcgcgccagaaaggtgtagccgatggcaacttcacgctggtcctcgttgaactcgtagtagcgggagctgcctgcagccttgccgctgtcgcggtcgatcacgatcagggtgctcttggccgccagtgcgtcgtcgtaccattggcgaaataccggctcctgatagcgggtcgagctttggtgctgagcccagatcaagggatcgcaggcgacgccatacagcgccgcgaagtccacggcctcaagtgggcgcagttcaagtgattcaccaacgagcaggggcgagaggggcagcgattgcatcagtgatctccaaaggacgaatgcatctgtttcgttgcgcgaggcttgctggatgagcaatatgctccggttgcatcacggctacgcgggagccggaaccttaacaaggctctggcaggcctgaatagcgcggcacggctgctgttggccgcgagtatccctgatcggcctgtggcgcatcacctggccaaccccgcgcaatactcaacaagcgcgcaatcgctctgagcacatcaatggatggcaacgcaacgatgaaataccccattagcctgttggcatgcacgctcttactggcctcgcccgcgtgggcgcagggcaagcgggtgattccgggtagcgagcaggtcgaggcgggttatctctcgtcgcaattgttgctgaagccctgtacggacgtgacgacgctgggcttcagcgcggacgacagcgccctgcacgatgccgccgccgcgcgccaggccttgcctgcgggaacctgcaacgacgcagcgctggtggagaaactgttcaaggcgcgctttgtgcagtacggctcgatcacgcaggtgttcaccccgtggcaattggcacgacaggtcgataggcagaaccgtgccatcagccgttgcccggatacccaatgcctgggccgcgagctggatgcagtgattgccgcgctggcgccggtgtatctcggcgcccgccgggtctggcccaggggcaaggggctgtgcgccagcgagccggtggacagcccggcgaaccaggccctggcggttctgggggccgacacccgtcagcagatcaatgcgcaatgcgcacaggagggggtgattgccacaacctgccacggcccccatggccaattgctgttcttgagctgtggcatgagcggcaatcaggtcaactcaagccaatggctttatcgtgccggcaaggcacacgccgagccgctgttcgccgtcgaggatggtcccctgggcgtactggcgacttcctgcaacggcatgcccgacctgatgaccagtgcacgggtcagtgccggggagcacaacgacaccttctaccggtacgacggcaaggcctatcggcaggtgtattcctacatcagcacctttgtcggtatcgatgccaacggcaatgacctggccatcgcccaggccggtgcactggccaaggtcgcgtgccgctaggggcgcgcattgctcacagcgcgcggcaaagatcaagattgacctccctggccgacgcccagaagatgcggcccttcgtatccagtctcgggagagggcatgcaggcgcatgacgcttacttcactatcgacgccgaggcgctgcaggaccgctccctggccgagctggccgccgggcagccgctgccgctgcgctggatctgagcggccaggacctgtcccgcatcagcctgcccggggcctggttcgaacgctgcctgctgaccggcgccgatctcaccgcggccaacctgatcaatacccgctggaccagctgccgcggcgcccaggccaacctgcgttctgcagtgcttaccgatgcccgtttcgaacgctgtgacctgaacaacacgcaatggcagcgcagcaaggtggcccatgcaagcttcgatggctgcaagctcaccggcgcccacttcggcgacgtgtcggccctgggcctgagcttttccgactgcctgctcaacagcgccatgctctcgggtatctcgttctacaagtcgcagttgcacaacattgatttcagcgaagccgacctgacctactgcgactttcgcaaggcggtgtttgtcgacggcggcagcctgtccatggcccgggtcaacaatgcccgcttcgatgacgccgacctgcgcgaagccagcctgcatgggctgcgcctggtggatgccaagctgttcaagggggcgatcatttcccgcagccaggccggcatgctcctggccgggctgggcctggccgtgctctgactagagcccggccgctgcgggttgggcctgcagcaattcgtcgagcacccccatgaaggcttccaccaactggctgcggccctgcttgcgggtcagtaccagcaactgggcggtggcgctggggtcggcgagggggcggtaagtcacgccttttttcgccagggtctgggtgcagcggggcaccagggcaatgccctggccggcggcgaccagggcgatgatcgaggtgatctgccggccgctggggcccgggcgcaatggcaggccgttgcgtcggtagagttgttcgatggcttcgttgagccccgagccgtagtcggcgggaaacaggatcagcggatgctcgctaagctcggccagagtcacttgcgcctgcgcggccagggggctgtcgctggagatcgccgccaccagcggttcgtcccccagggacagggactggatatcgctctgctcgctcggcggttgcatgcggctcatgccgatgtcgatgcgcccgtcgacaatctgcgaccactggctgaccgaagcaccttccaccagggtcaattgcacgtccgggaagcgctgggcgaaggcctggatcgcctggctgaacaggtccgagagggcgatggaactggcgtagcccagggccagttgcccggcgcaacctgcagccagcttgccggccatgacctgggccaggtccacctgttccagcacggcccgggcgtacggcaggaaatgccggccctgggcagtgagggtcacggtgcggctggtgcggtcgaacaggcgaaaccccagttcggtttccagggccgaaatctgccgggtcagcggcggttgcgccaggtgcaggcgcagggcggcgcgaccaaaatgcagctcttcggcgaccgtcagaaaataacgcaacttgcgtagatcgagcatcctgttccctgggtattgatcggtcggaattcggtattggtcttggcttggcgacgtattttataaaggcctttggtcataaaacgagaggtttcatgaattcgcgtcagcattgtgcgcgcatcgccctgtttctgtgtggttgtgcggcatttctcaacctgtatgcgacccagggcattctccaggaatttgcccagagctttcaggtgagcatccgggaagccggctggagcatcaccgtcaccaccctggcggttgccctcactgcgccctttgtcagccgctttaccgggcgtttcgatcagcgccgggtgattgtggtggccgcgctgttgctggcggtgccggcggggctggcggcctatgccggcagctttacccaactgctggtctggcgcctggtggagggcatgctgatcccggtggtgttcgccaccagcgtggcctatatcggcgaccgctggagcggcggcaccgtgaccgaagtcaccagtctgtatgtcgccggcaccgtgctgggcggctttgccgggcgttttgtcaccggtttcatgaccgaatacgtgggctggcgcgaagcctttgccttgctcgcgggcctgagcctgctgatcggggtggccattcatttgctgctgccggccaacccgcccctgaccgcgccccgggcatccggcggcctcagtgccctgcgcgagctgtgccggcggcccttgctggcagcctacggcgtgggcttctgcgtgctgttctcgcaagtggccacctttacctacgccgggctctatctgagccaggcgccgttcgacctgggggctgctgccctgggcgcgatctacagcgtgttcctgctggccctggtggtgatccccatcgccgggcgcctgagcaagtcgcggccccaggccgagctgctggtggccgcggcgttgctcggggtctgcggttcggccctgaccctgctgccgtcgctgtggctgatcgtgctgggcctggccctgagttccaccggagtgttcctggcccaggccgccgccaactcctttaccaccgccaccgcccgccacaacaaggccggcgccgtgggcctgtacctgacctgctactacctgggcggcagttttggcgccgtggtcccggccctgctctggagccactggggctggcagggctgcgtggcgctgatcgtgctgttccaactgctgtcgctgctgattgcctggggcggctggaagcccctcgaaccccaaaccccggtcgcggctcgcgaaccgtccaacccgatagattcatcatccaacaaggaatacgtgaggtgaccatgaataacgtactgacccggcagagccagttgctgaagattgtcgaacctgtgttgaacgacatgccggccagtgtcttgaagcatgcgctgttcgaggcgttctggagcgaagagggcgccctggtggacatcgagcatgccttcgagaagctcacctcccgtcgccacgagccggcggtgctgcgcaagttcttcgccagctggtcgaagaccaacaactccgccaccagtgtgtccgggctggccaaccgcatcaccttgctggcccgctccgggcagggctcggaagccgccgacggcctgtaccgggcctgcggcagcctgcagcggatcaccgatgaagacctgggcgccctgggcaataccctgcattcggacctgttctacaacatggccacgccgatctgcggcgatgaccagtggctgctcaaggaaaactgcctgccctcggcccagggcttcaaggactggacggattttcgccgcctgcgtgaccgcgacctgctgcagggcctgctgaccaccctgatccacgaggtctacacccacggcgaagtggagtttatccacccgctgtacaagcagtggttcgtccgcgacatgggggtgccggcagagaaggcgcgcaccaccgtggcctgggtcacggtgcacaccggcggcaccgagagcaatcacttcgcccatgcggtggcggcggtcaacgagttcgtcgatgccatgcaggtgcgggtcgacccgcaggccgcgcgggaaatcttcgccgaatacctgcagcgcaaggctcgggtaatgcgcgactgtgcccagctgttgcgttgatggctccggcccggccccggcaacggggcttgtggctgacccaggctcgcctatcctgttgctgattgacccttccttttcattttgcagcctgcctcgacaatgaatgacctgaaacatcacgcctccccggaggcattgccggcgtccgatgtgtcggtgcgccgccgttccctggtggccggttgcggcgcccacgcggtgcatgacggcctgactgacgtgatctatgtactgctgccgatctggcaggcgcagttcgccatgagctacgcccagatcggcctgctgcgcggcgcctattccgggatgatggccggcttccagttgctggccagccgtggagccaagcgctggggacgcgagcgcctgctggtgggtggcactgccctggccgggctggcctacctgctggcggggcaggccggggggctgggggtgttgctcctggcgctgctgctgggcggcctgggggccagtacccagcacccgctggcgtcctcgatcatcaccgacacccacgaggctggcggcggggtcaaggaagcgctgtcccagtacaacttcgccggggacatcggcaagaccctgattcccgggttggtggggctgctgctgaccgtgatcagctggcgtgccagcgtgaccctgatcggcctcctggggctggccgctgcgctggcgctgtggtggctgatcccgtcccgggcggcgaccctggccatgcaggaaaaggcgccgaagacggtcaagggccagggctcggtcggcggcctgcgggcactgatcctcaccggcaccctggacagtgcggtacgcatgggtttcctgaccttcctgccgttcctgctcaaggccaagggcgccggcaccgccggtatcggcctggccctgaccctgctgttcgtcggtggcgctttcggcaagctgctgtgcggttacctcggggcgcgtatcggcatgatgaagaccgtgtggatcaccgagttcaccaccgccagcctgatcgtgctggcggtgttcctgccctatgtggggttgatggtgatgctgccgctgctggggctggcgctgaacggcacctcgtcggtgctctatggcgcggtgccggacctggcgggggcgggcaagcgcgagcaggcgttctcgctgttctacaccggcaccatcggcggcggggcactggcgccggtgctgttcggcagcctcggcgacgtcaccgggattcccctggcggtgatggccctggccgcgaccctgctgctgaccctgccgctgtcctggtacgtgcagcgtggcctggacgccgacgcccggtccttgcccagccccggctgaggtcccgccaatgcattgaaagcgcaggcccggagcgggattgtccgggcctgctgcttgctggtggagcgctagccgcgcaggcgccattcgggccccgcggcaggggccggcagcggcgctgccagggtcctggccagttcgacgaagcgctccaggcagcgcgaccccaggggcgtgcgccaactgaccgtctgttccagcacatagggctcggccagcggcacatagaccaccccgggacgctgcagggtcgcggtgtagtcgggcaccacggccaccccgcgctccgccgccaccagggacaccagggtggtcatctgtgacgccgtgggccccaggcgcgggatgatcccgctgtgggcgcagaggtcctccagggcggtgttcagggccgatccgtaggcgctcggaaaggtcacgaaggattcccccgccagttcgcaaaaccccaggctgctcctgccggcgaaagcgctgcgcgacggtaccgccaccaccagggaatcgcgcgaactcaccagcgactgcaaaccgtgggccggcaagttgggcgccccccagccaaagccgatatccaggctgccatcgatcaactgctggcgctggctggcggtggacacctcgcgaaacgccagctccacctccggcaggctgcggctggtgttgcggatcatctcggagaaagcgttggacaggggaatcgagctggcatagccgatgacgatacggccgatggcgccatgggcgatcttgcgcgcggtggcctcggcctcgccgacggcactgatcaccccccgggcctgctgcaggaacagttcgccctccggggtcaggcacacggtgcgggtcgagcgttcgaacaggcggatgcccaggcgcgcctccagggcggcgatatgccgggttagcggcggttgggcgatccgcaggcgcagggcggcgcggccgaagtgcaattcatcggcaacggtgacgaaataacgcagacgacggagctctagcatttcttgtttttccagtatcaatcacgacgcaaacagtattggccgctatttgcgactgtcatcagaatgtaagacgaccagcctagaccagctttccaggttgccggaggcgcatgggtacaggctcggttcactctgttttccgattgttttcgcaggtacaaaaatgacaagcagcgaagccacggccgtccgtttttccttgtcccagcgtgcgctgggcctcagtgccccaggcacctcgagcatgcgcagcaaagccaacgccctgaaggaaagcgggatcaaggtgatcaatttcgccgccggcgaattgtccttcgatgcctgcgaggacatgcgtgccggggccatcgaagccatcgctaatgcgcgcaaccgctacaccccgcccatcggcctgccggccctgcgcgagaagctcgcgcaacgggtcagccagcgcaccggggtggagttcgccgccaatgaagtggcggtcaccgccggggccaagcaggcgctctacaacgcctgcatggtgctgctcaaccccggggacgaggtgatagtgccgaccccgtactgggaaacctttcccacccagatccgcctggccggcgccaccccggtgtgcgtccagacccgcgccgaccactaccggctgacggtggacgcggtgtgtggcgcgctcaccgagcgcacccggatgatcgtcatcaacacccccaacaaccccaccggcaccgtctatgaacgcgagcaactgctggccatcgcccagttggcccaggagcggcaactgtgggtgatgttcgacgagtgctatcgcggcctggtgcgcgaaggccacgagcaccacaacatcctggcgctgtgcccggcattgaaagggcagacggtgctgatcgattccttctccaagagccaggcggtgaccggctggcgggtcggctacgcctgcgccccggcgccggtgatcgcggccatgcacaacctgcaggggcacaccacctcaaaccccagcagcctgtcccagtacgccgcgctcaataccctgaacgccgattcgtcgcactttgccgacctggtcaacccgttcctgcaacgccagctgcacgccgcacgcacctacctcgaccgcctggaaggggtgagctatgcgccgccggaaggcgccttctacctgtatatcgatgtctcttcccggctcggcggctactaccagggcgaagcggtgcgcgatgtggatcacctggccgacctgctgctgaccgaggcgcacatcgcggtggtgcccggttccggttgtggcgatccccactgcatccgcatttcctacgccgtggaatatgaggaactgatcgaaggcctgggccgtttcgcccagttcatctcggcgatcgtcatcgactgaagcggctcccggcagcccagcccccagggggatcaagcccgttcccgagcccgccgtggcccgggccggcgtcgttcatcttgttgtccgtgaagtgaagaacaattacaggagaaaaacatggagagttcccgcaaggcgttcatcggctacgtgcaccaggccctggtggatgtcgaggaccgcaacctggtggaggccctgctgacgggctttgaaaaccaccccgacaagctggacggctactgcctgacctaccagcgcatgacctcgcgcaagtggtccgaggactcgctgtgcacctttttctgcggctggcgcagcccggatggcgcggctcatgcggtgtccagcatcatcgtgcggctgctgcaggagtccgaggacctgcccggtgatgacaacaagctcaagctgctggaggcggcccggcactgcggcgagatcatcgtcgaggacgtcggcctgggcgagatgcacggccatccccaccattccaagctctaccaccgcatggcctcggccatctgcggctcggacaactggcgcctgcaggacaagtacctgaaccccatcaccaaggagttctccacctgggtcggcgagaagcgcccgctggccccgaacctggtggaagccctggagatgatggccctgaccgagctgttcaacaccggcgagtacaacctgatgacaccgctgtggaagaactggctgcgcgaatcctgcggttaccccgccggggaagccaaccgcatcgccggcttcctcagcgttcactgcggcgccgtcgaggcccggcatttccgccacgccaccgacgccctgaagctctacacccaggccaccggccagcaggtcaactaccggcgcatcgctgcgctgtccgatgagtacgtgctgcgcgcctgccagcacctggagaagatggcctccgtactcaaggaataagcgcctgccaaccaggccgggcagggcgcctgcccggcaccgcaggccggaccctcattcatttattggagaactacccacatgcgtcttgaatccgactcgatgggttccatttcggtaccgtccaacaagtactggggagcccagaccgaacgctccatccacaacttccccattggccgtgcacgcttccagtggggggcgccaatgatccgcagcatgggcatcctgaagaaggccgcggccctggccaacctcaagctcggcgagctgccggaagccattgcgctgcccatcgtcaaggcggcggatgaggtgatcagcgggcaactggacgagcatttcccgctggtggtgttccagaccggttccggcacccagtccaacatgaacgccaacgaagtgatcgccaaccgggccatcgagatgctcggcggcgagctgggcagcaagaagccgatccaccccaacgaccacgtcaaccgtggccagtcgtccaacgacaccttccccacggcgatgtacatggtggtggtgacggagatcttcgaaaccctgctgccgggcgtctcggtgctgcgcgacacgctgttcgagaaggccgagcagtaccgcgatgtggtcaaggtcggacgcacccacttgcaggacgccacccccatcaccctgggccaggagatcggtggctgggtcgagcagatcgactatgccctggcggccatccagcacaacctcaccggcctctacgacctggccatcggcggcacggcggtgggcaccgggctcaactcccacccgcagttcggcgacgagtgcgcacgaatcatcgccgagctcaccggctacccgttcaagtcctgcgccaacaagttcttcgccctgtcggcccatgacgcgctgatcaacacctcggcggcgatccgcaccctggccatggccctgatgaagatcgccaacgacgtgcgctggctggccagcgggccgcgctgcggcatcggcgagatcgacattccagagaacgagccgggctcctcgatcatgccgggcaaggtcaacccgacccagtgcgaggcgctgaccatggtctgtacccaggtcttcggcaacgacgccaccgtggccttcgccggcagccagggcaatttccagctcaacgtctacaagccggtgatggtgcacaacgtgctggaaagcatcaccttgctggccgagtcctgccgggccttcaacgaccactgcgcggtgggcatcgagccgaaccgggcgcgcatcgaggccaatatcgagaagaacctgatgctggtgaccgccctcaaccgccacatcggctatgacaaggccgcgatcatcgccaagaccgcccaccacgagggcaagtcgctgcgggaagtggcccagcagctgggctatgtctcggcggcggatttcgaccgctacgtggtggccctggacatgacccatccctgagcgaacccggtgttccccaaccacaagaggcgtgtgaatcccatgcaggaagctgatattcgccgaaccaacctgagccagttgctgcgccgcaagcgctgccttcgcgtgctcgaagcccacagcccgatctccgccctgctggcggagcaatccaggctggagcggggcagcggaccgagcctggtctacgacgccatctggtccagctcccttaccgactcgacccacaagggcctgccggacatcgagatcctctcgccgagcaaccgcttgcacggcatccgcgagatcttcgatgtgtgttcgttgccgatgattttcgacggcgataccggaggcaaggccgagcacttcgccatccacgtgaagatgctggaccgggccggggtctcggcggtggtgatcgaggacaagtgcgggctgaagaagaactcgctgttcggcaacgcggtgagccagctccaggactccatcgaggagttctgcgaaaagatccgcgtgggctgcgccaaccgcagccacgaggatctgctgatcatcgcccgttgtgaaagcctgatcctcgacaagggcatggacgatgccatcgagcgctgcctggcctatgtcgcggccggcgccgacgggatcatgatccacagccgcaagaaggacggcctggagatcctcgagttcgcccggttgttccgccgccactgcccgcgggtgccgctgatctgtgtgcccaccagctacgctcacttgagtttcgacgagctggagggggccggcttcaacgcggtgatctacgccaaccacatgctgcgcagtgcctacatggccatgcgtgatgtggcggtggggattctcgagcatggccggaccctggaggtggagccgcggtgcctgggcatcgacgagatcctcgacctggtgccgggcacccgttaggcactcgcggccaacccccttcgcagggccgtggaacgggcgtgctttatccgattccactccgagattcggaacgttctgaattcagaattttcctacagacgctgtggggggcgctgtctagactcgctccccgttcgcccggccagctaccacaggcaacacgctgaatggcggtggttggtcagggcgggctgcaacacggctgtacctgcctgcccggtccatgtttcgcctactgcccaggctgaccgggttggttctacagcgtggggcactcatggccccggtccccgcatccccccaacctctcatcgccccagccccctgatacccggccatcgccagtggctcagggcaaggtctgggcgcgggccaggctcggtgcctgcgcgccatcctcccaggcaccacccagggcccggaacagatttacctcggccagcaactgcgccaggcggtcgttgatcagcccttgctgggcgctgaacagcgagcgctgggcgtcgaggaacaccaggttgctgtcgacgccgatgcggtagcggtgctcggccaggttgtagtagtcctggctggcctgcaccaggctgtgctgggcactcagttgctgctggaaggtctggcgcgcggccaggccgtcggcgacttcctggaaggcggtctggatggccttttcatactgggcgacgctgatgtccttctgcagccgggcgtaatccaggctggcccgcaggctgccggcattgaagatcggcaggttgatctgcggctggaaggtccaggccccggcaccacccttgaacagcccggacagctccttgctggcgctgccggcgttggcggtcaggctgacgctggggaagaacgctgcacgggccgcgcctatattggcgttggcggcctgcaactggtactcggcctggagaatgtccgggcgccgctgcagcaggtccgagggcaggcccgcgggcacctgggccagcatgccctcggccagcccgcgccggggcagggcgtcgggcagcggcgcccccaccagcagcgtcaggttgttcaggtcctgggccacctggcgctggtagcgggccaggctggcgcgggcactgtcgacgctggtgcgggcctgggccacatccagggccgagatcttgccggcgctgccattgcgggcggtcaggcgcaggctctgttcgtccgcggccagggtcttgcgggtcagctccagcaactcctggtcggcacaccagctcaggtaggccgtggcgacattggccaccaggctcaactgcgcgctgcgccgtgcctcttcgctggacaggtaggtcatcagcgcctgctggctgaggctgcggatgcgcccgaagaagtccagctcataggcactgatgcccaggttggcggagtagctggagctgatcagtgccttgctgccggtcatctgttccggcaggcgctggcgcttgcccgagacgccggcgctcaccgccgggaacaggtcggcacgctggatgcggtactgggcctggtaggccgcgacattcagcgccgccactcgcaggtcgcggttgttctgcagggcgctgtgaatcaattgctgcagcaacgggtcctggaacagtccctgccagtcggcccccgggttgccggcagcgggggcgaaggctgcgccctgtgggtactggggcgccacgggagcggtcgggcgctggtagtccggggccagggagcagccgccgagggccagggccagggccgccagggtcagtggggagttacgcattgatcagcctcgtgacaaggacatcctgatggcggacagccaggggcgtgcgggcggtttcaggcgccgaccatccacttggccagcccgtgacggccgctgaccccgagcttggcggcggcgcgcttgaggtaggtttccaccgagctgttcttgacgctcaggcgctgggccatttccggcacggtgccgccggtcagcaaccccaggcagacttccttttcccgcgccgagagggtgatgtcacccagggacaggcgctcatcgaactcccgttgcagcggtgtctgctgcaaggcatcggcgggcttggcactccagctgcgcaggttcttcagggcctgttgccggtgcagctgggcgtggcgttccagcaacggcagcagggtctgcgacaggctcttgagaaacgacagttcggacagggaaaagccgccctggtcgggagcccggtagagggcgatgacacagcggcgattggctttgcgcgagaccaggtggcattggtgcgcggtggtgcgggaagggccgctgcgggcctgggcattcatttgaatcagcagcgagtcgtccatttccagcatttgctgcagcagcgggtcatggcgtcgcggcggctccggcagtggcaggtccttgtgcagcccggcgcgccccagcagggtaatggccagggggctggcctgacgttcgtccagggtccattcgctgagcaccagttgggtcagtggcaccagcttgtccaccagttgcagcatttcgctggcgaactgcgggtggccggagctggaaatcaactgccccagttcgaagtagaagtgcggggtttccaggttcgcaatactgctggtcagactcatatccttatcatccttgatcgagacaaatcgctgatcgatgcgcgcttgccgcggcatgccgtggcagattcccgatgaacctgatccagttcccgggcgttgatttaagcctatggctttgcctggcgtctgtagggggaaaccgggacatcatggccctgccagggcctcgccaaggctctgggtcaagcccttgcgacggccccgtcgcgcccgccggcggggtgtttcccggcgtcaaggttgcccgaggggcaagcctgcggtggaacgcaaaagccctccgcaggccatgagcgtcgttcacgcgtagccaatttcctacagccgattcagctgatcagcagagctgctgcaacagcccggcgcggtgctgcccgaggtggccgaccggggccggaactgtccgggtttcgggtgacatacaggccctggggccgatgcttaaatccgcggacgccggtgcccgggcccatcacatgagaaggatcttatgacgcatttttccgccgcagccgtgggcgctttcgtcgcgccagtcaccccagccagccactgcccgccatgggccgggcaccccgcccgggggcgcgcatgaacgccggtccggtacaggcccggggcctgccgctgaccgccgggcagcgcgatatctggctcgaccaactgagccggggcgactcgccgctgtacaacatcggcggctatgccgtgctcaaggggccgttccggcccgagctgatgcagcgcaccgtggagctgctggtggacaagcacgacgtgctgcgcacggtgctgtctctggagggcagcgtcgacggcctgcccttgcagcacttcgaccggcagtggccggtggagctggtgttccatgacctgtccgcggcggccgatccgctggcggcggcccaggcgcagatccgccggcagatgcagcaggcgttcaagctcgacggcgaacggttgtgccgcttctcgctgttccgggtgggccccgagcatcacctgatggaagtcgaggcccatcacctgatcctcgacggctgggggttcgcccagctgtcccagtccctgggcgggctctacagcgccctggccagcggcgaggagccccaggccacggcgccgtcctttgtcgacttcatcgaggacgacgcccgctaccaccagtccagccgctatgcccgggaccgcgactactggctggacaagtaccagagcctgccggagccggtgctgcgcccgcgctaccggcccgccgccgacgccgcgaccccgggggcgaccctggtccaggcgttccccgcggccttgcacgaacgcatgaaggccctgggcaagagcaccggggcctcggcctttcatgtgctgctggcggcgctgcatgtgtacttcacccgcaccctgcaacgggacgaatgggtcctgggcatgccagtgctgaaccgccccaatgcggccttcaaggtgaccctgggttccttcacccaagtcagcgcggtgcgcatggacttcggccgcgaactgagcttcgaggccctggtgggggcggtgcgcgatgtactgaaacaggacttccgccaccagcgctttcccctgagcgaactcaaccgcgccctcggcctgctgcgcgaagaccgcgcgcagctgttcgagctgtcgctgtcctacgaattggaagacctggactaccgctacggcgaggcggcggcctcgtcggtgaaagtctccaaccgccacgagcccacgcccctggccatccacctgcgcagcaacagcttcgatgacagcgcctggatgcactacgtctacgactcggcgtatttccagcccgaggaaatcgaggccctggccgcgcgcctgctgcatgtgctggagcaggggctggacaatgtgcaactgccggtggcccgcttctccctgctgcctgccgccgagctgcgccgcttgcaggcctggaacgccaacgaacagccagccgccgccgaacagcggattcacgcccgggtcgcggcctgggcccggcgcacgccccaggccattgccctggtcgcccagggccaggccctcagctatgaacagctcgaccgccgggccaacgccctggccctgcacctgcgcgagctgggagtggggccggatgcgcgggtggccattgtcgcccggcgcagcccggaaaccctggtggggctgctggcgattctcaaggccggcgcgggctatgtgcccctggacccggcccatccggctgagcgcctggccttcctgctgcacgacagcgcaccgcgggcggtgttgacccagggcgccctgcgccagtgcctgccgacgctgaacgtgccgctgatcgacctcgacaacggccagtggctggacggcccgggcgagattgccgaggccgccgggctgaccgcggagcacctggcctacgtgatctacacctccggctccaccggccagcccaagggcgtgatggtggagcaccagagcctgtcgaacctggtggactggcatggccgggctttcgacctccaggctggcagccacacctcatgccttgccggtttcggcttcgatgccatggcctgggaagtctggccggccctgtgcgccggggcgaccctgcacctggcgccggtcagcgacggccccgaggagctggaccgcctgctggcctggtggcgcgcccagccgctggaggtgagctttctgccgaccccggtggccgagtacgccctcagccagcaactgggccacccgaccctgcgcaccttgctgatcggcggcgaccgcctgcgccagttgcctgctgaccccgggtttgccgtgatcaacaattacggccccaccgaggccacggtggtcgccacttccggtgccatcgaggccgggcaggccctgcatatcggccggcccatcgccaatacccggatctacctgctggatgaacagcagcaactgctgcccatcggcgtgcccggcgagctctatgtgggcggtggcggggtggcccggggttacctcaaccgcgatgggatgaatgccggacgcttccttgcggaccccttcagcgaccgtcccggcgcgcgcatgtaccgcagcggcgacctggctcgctggcgggaggacggcagcctggaatacctggggcgcaacgacgatcaggtgaagatccgcggcctgcgcatcgagctgggggaaatcgagacccgcctgggggatcacccggcagtgcgcgaggcgctggtccaggcccgggacggccagttgctggcctggttcattccgcgccaggcggtcacggccctgcaactgcgggaatttctgcgccagcagttgccggagtacatgttgcccatggcctacgtaccgctcgacgcctggccgctgaccggcaacggcaagctggaccgccgggcgctgccggcgccggggcccggggacctgatcagccgcgcctacgaggcgccccagggcccgctggaaatcaccctggccgggctctggagcgagctgctgcaggtggagcaggtgggccgccacgatcacttcttcgaactgggcgggcattcgctgctggccctgcaactgatccagcgcatggcccaggccggcctgcaggccgatgtgcgggtgctgttcggccagccgaccctggccagtctcgctgcggcggtgagcaccgggcaggtcgtcgaggtcccggccaaccgcgtcccgcccgactgccagcgcatcaccccggcgatgctgcccttggtggagctggaccaggacagcatcgaccgggtggtggcctgcatccccggcggcgctgccaatgtgcaggacatctaccccctggcgccgttgcaggagggcctgctctatcaccacatgaccgccgcccgcgatccctaccagcagcacgcgctgttcgccttcgccgggcgcgaggacctggacgccttcgccctggcgctgcaggcagtgatcgagcgccacgacatcctgcgcaccagcctggtctgggacgtgctggagcagccgctgcaggtggtctggcgccaggcccggctgacggtgcaagagtggcaggccgggcccggcggccatgtagcggagcgcctgcgcgagcacttcgacccgcagcacaacccgctggatatccgccaggcgccgatgctggccctggcctgggccgaggaccgggccaatgggcgctggatcggcctgctgcgcttccatcacctggtcaacgacgccaccagcaccgcggtgctgctggcggaaatcgccgcccatgtgcaaggccggcaagccagcctggcgcctgcctatgcctaccgtgactacgtggcccgcacgcgcctggggcaggccgcccacaaggcgttcttcgaggccggcctgggcacggtggacgagccgaccctggccttcgaactgcaggaacggcccggggaacaactggacctgcagcacgccagcggcctgctggaagccgagcttggccagcgcctgcggacccaggtgcgccagctcggggtcagcgccgccagcctgtttcacctggcctgggccctggtactggggcgtaccagcggccgtgaggacgtgctcttcggcagtgtattgctgggccgcctgcaagccggagcgggggccgaccgggccctgggcatgttcatcaacaccttgccactgcgcctggagctggcggggctgacggtggccgaggggctgggccaggcccagcagcgcctcagtgccttgctcgcccacgagcaggcgccgctgtccctggcccagcgctgcagcgcggtggcggcgccgaccccgctgttcaatgccttgctcaactatcgccacaacctcggcggcgacctgttgcagggtttgcctggggtcgagttgctgagcagcgaagaggtgctcagctacccgctgatgctggcggtggacgacctggaaccggggttccgcctgggctgccgggccccgcggcggatcggcgcgcaacgcctgctgggctacctgagcaccgctctcgaagcgctggtgctggccctggaacaggcgccgcagacgccgttgcacagcctgtcgatcctgccgccggtggagtcgcgctacctgctgcaaacgctcaatgccacccgttcgcaatacccgcgggagctgaccctgcatgccctgttcgaagcccaggtgcggcgcacgcccgaggccattgcactgcaggccggcgcgcggcaactgagctaccggcagctcaaccagcgcgccaaccagctggcccattacctgcgcgagcagggtgtggggccggatgtgcgagtcgggctctgcgtgcagcgcagcccggaactgctgatcggcctgttgggcatcctcaaggccggcggtgcctatgtgcccctggacccggactacccgctggagcgcctgcgctacctggtgcaggacagccagccactggcggtgctggtgcaccagccgacccgcgagctgctgggcccgctgcccctgtcgctgatcgacttcgaccgcagtacctgggagcaggcgccgcagcatgatcccgtggtggcagggctgacggtttcgcacctggcctacgtgatctacacctcgggttccaccggtacccccaagggggtgatggtggagcaccgcggcctgggcaacctgatgcactggagttcgcagctgtgtggcgagcgggcccggggcaccctgctgcaaaaggcgccgttcagtttcgacggctcggtctgggagctgttctggcccctggtcacgggcatgcgcctgttgctggcgcggcccgacggccagcgcgatccgctgtatctggcgcaattggtgcgcgaggagcaggtgagcatgatcaagttcgtgccggccatgctcctgcaatttctgcagctggaagaggccggccagtgccacagcctgaccgatgtgttctgcggtggcggcgagctgaccgaggcgattgcccggctgttccgccagcgcctgcccggagcccgtttgcataatgtctacggccccaccgaggccaccgtggacagcagcgcctggaccctggagcccggggcagcggtgccgccggtacaactgccgatcggcaaggccatcaccaacacccggctgtatgtgctggatgcccatgaccagccggtgccccagggcgtcagcggccagctgcacattggcggggtcggggttgcccggggttacctgggcttgccgcagttgcaggccgagcgctttatcgacagcccctttgtcgccggcgaccggctgtaccgcagcggtgaccgggtgcgctacaacgctgacggcgacctggagttcctcgggcgcaacgacttccaggtcaagctgcgggggttgcgcctggagctgggggaaatcgaggcgcggctggccagccacccggccctgcgcgaggtggcggtgctgatgcgtggcgagcgcctggtggcctatttcagcctgcaccccggggtggcggcgccggggatcgaggcgctgcgggcccatgtcctggagcgtctgccggagtacatggtgccggcagcctatgtgcaactggcggcgctgcccctgagcgccaacggcaagctggcgcgcgatgcactgccggagccgggcctggaagcggtgctgagccgcgatttcgagccgccccaggggccgcttgaaaccaccctggcgcggctctgggccgaggtactgcaagtggagcgggtagggcgccacgaccacttcttcgaactgggcgggcattccctgctggcggtcagcctggtggcgcggatgcgccaggaaggcctgcacgccgatgcccgcctgttgttcagccagccgaccctggcggcgctggcggcccataccctgggccagttgcagcgcctggaaatccccgccaccaccatcccccagctcaagcgccagcggcgtctctgaggccgctgcaagtgcttttccccgcgccggccccagggccggcgcgggccgtggtgcctcagcgggacaggccccgcgccatgtcccggcttcccatgtcagccccgctgcggcggatgttttagcgtctgcgagctaccagcaagcctggcgggctcactgtccctggtgctttttgcgcaacggcgcgacagccccgcgcggtgcctggcagcggcgcttgtcgcccgtccgggcagtggggcgccggtgaaaggttgcatctgaaacaaggacgttctcttcaaccgtttccgaattctcaggcaggttacaagatgcagttcagcgaattgatggcagtgctttcgaccctggcgatccgtcttcaacgggaacaggaggacctgctcgtccagggcgatgacgaggcgctggacgacgggctctgggacagcctggtgcagcacaaggcccggctcctggaactgctcgacggccagggcggcagctggctgagcccggccttcaggatcaccccggacatgctgcccctggtgcagctggacccgcccgccatcgagcgtatcgtcgccgcagtgcccggtggcgccgccaacgtgcaggacatctaccccctggcgccgctgcaggagggcatgctctatcaccatttgtccgccgcccagggcgacccctatgtgctgcaggcgcgcttcgcctttgccagtgccgagcgacggcaagccttcgccgaagccttgcaatgggtcatcgaccgccacgacatcctgcgtaccgccatcgcctgggaaagcctggatgagccgttgcaggtggtctggcgccaggcgccgctggtgatcgaggccgtggcgctggacccggccgacggcgatgtgctcggccagttgcaggcccgctacgactcccggcactttcgcctggacctgcgtcaggcgccgctgctgcgcctggtgcacgccgaggacccggccaaccagcgccaggtggcgctgctgctgttccatcacctggccctggatcactccgcgctggacctggtacgccaggaaatccacgcccgcctgcaagggcacagcgagcgcctgccggccccggtaccgtttcgcaaccatctggcccaggccctggcaagccgcagcgaggcggcccacgaacgcttctttcgcgagatgctcggcgatatcgacgagccgaccctgccgtgcggcctgggggatgtgcagggcgccgggcgggtgatcgaagaggcccggctgaccctggaccgcgagctcagccagcgcctgcgggcccaggcccggcagctgggggtgagcagcgccagcctgatgcacctggccctggcccggatgctgggccagttgtcggggcgcgaagcggtggtgttcggcaccgtgctgctgggccgcatggaggccggcgaaggcggcgagcgggccctgggcatgttcatcaacaccttgccgctgcgggtggatgtcggcgcccagggcgtgcgcgccggggtcctggccacccatcagcggctcagtgccttgctcgcccatgagcatgcgtccctggccctggcccagcgctgcagcggggtcagtgcgccgacgccgctgttcagcgccatgctcaactaccgccacagccacgccgccgatggcgcccaggtgcaggaggtggccccgggcatccaggtgctgggcgccgaagaacgcaccaactacccgctgaccatcaacatcgatgacctgggagaggatttttgcatcaccgccctggtggatcggcaactgggcgcagagcgcatcgccggctacctgctgaccgccctggagagcctggccctggccttgcagagcactccgcaggcaccgctgtacagcctggagatgctcccggccagcgagcgccgctacctgttgcagggcctgaacacgccgctggggcactacccggacagcccgctgatccaccagcaggtggaagcccaggcccgggtccagcccgaggccccggcgctgctgttcggcgagctgcggctgagctacggcgagctcaaccggcgcgccaaccaggtggcccaccgtttgctggcgctgggcgtacgcccggaccagcgggtggcgatctgtgtcgagcgcggcgtggaaatgatcgtcggcctgctgggcatcctcaaggccggcggcgcctacgtgcccatcgacccggcctacccccgcgagcgcatcgcctacaccctgcaggacagcgacccggtggcgctgctggtgcaggccggcacccagtccctggtggccgacctgcgggtgccgctgatcgacctcgacagccgcaccctggcccatgaagcccaggacgaccccgaagtgccggggctgaccccggcccacctggcctatgtgatctacacctcgggctccaccggcctgcccaagggggtgatggtggagcaccgcaacgtggcccggctgttcagcgccacccgcgactggttcgacttcaactggcgggacgtgtgggcgctgttccattccttcgccttcgatttctcggtgtgggagatctggggggcgctggtgcatggcggccagttgctggtggtgccccaggccgtcagccgttcgccggatgactgctaccggctgctctgcgaagcccgggtgagcatcctcaaccagacccccagcgccttccgttcgctgatcgcggcccaggaccagagccccctcaagcactccctgcgccaggtgatcttcggcggcgaggccctggaaccggggatgctcaagccctggtatgcccacctggaaaacgtcggcacccagctggtgaacatgtacggcatcaccgaaaccacggtgcacgtgacctatcgcccgttgcaggcggcggatgcccagctggtgggcagcagccccatcggccggcgcattcccgacctgcaactgtatgtgctcgatgcccaccgcgagccgctgcccagcggcgtggtgggtgagttgtatgtgggcggcgccggggttgcccggggctacctcaaccgcgatcagctgaccgccgagcgctttatcgccgaccccttcagccacgagccgggggcgcggctgtacaagaccggcgacctggcgcgctggcgcagcgacggcagcctggagtacctggggcgcaacgatgaccaggtgaagatccgcggctttcgcatcgagctgggggagatcgaggcccgcctggcggcctgcgacggcgtgcgcgaagcggtagtgatcgcccgggaagacaccccgggggacaagcgcctggtggcctatgtgatcccccggccgggggccgctgccagcgctgcgcaactgcgcgagcaactgcaacagagcctggccgagcacatgctgcccagcgcctttgtgacgttgcaggcctggcccctgacccccaatggcaagctcgaccgcaaggccctgccggcgccggacagccaggccctggcccggcgtgaatatgcagcgccccagggcgaggtggaacaggccatcgcgacgatctggcaagagctgctgggcttgccgcaggtcgggcgccaggacaacttcttcgagctgggcgggcactcgctgctggcggtcaagctgatggagcgcatgcgccaggtggacctgtgcgccgatgtgcgggtgctgttcggccagccgaccctggccgcgctggcggcgaccgtgggcgggcagcgcgaggtgcaggtgccagccaaccgcatcgccgccgattgccgccacatcaccccggacctgctgcccctggcggacctcgatcaggccgccatcgaccggctgctggcgcgggtgcccggcggcgccgccaacgtccaggacatctatgctctggcgcccttgcaggcggggatcctctatcaccacctgagcagcgccgagggcgatccctatgtgctccaggtgcagttcgaatttgccggcgaagacgccctgcaaaccttcaccctggccttgcagcaggtcatcgcgcgccacgacatcctgcgcagcagcatggcctgggaaggcctggaacagccggtgcaggtggtctggcgccaggcgcccctggacattcaggtggtcgagacggacccggcccagggcccggtgctggagcagctgcaggcgcgtttcgacccccgtcgctaccgcctggacctgagccgggcgccgctgctgcaactggcctgcgccgcggacccggggcagcagcgctggctgggcctgctgttgttccaccacctggccctggaccacagctccctggaggtgttggtggaggagatcagcgcggtgctgcagggcgccgccgcgcagttgccggcgccggcaccgtaccgcaactatgtggcccaggcgcgcctgggccatggcgaggagcagcaccaggcgttcttccgcgagatgctggcggacatcgacgagccgaccctagccttcggcctgcaggacgtgcagcgcgacggcagcggcatccgggagcgtcaactgcgcctcgacccggcgctgtgccggcgcctgcgagagcaggcgcggcacctgggagtgagcctggccagcctgctgcacctggcctggggccgggtgctggggcaactggcgggccgcgatgacgtggtgttcggcaccgtgctgctgggaaggatgcagggcggcgccggtgccgaccgggccctgggcatgttcatcaacaccttgccgttgcgggtcagtgtcggggagcagggtgtggccgaggccttgcgcgccacccacgcccggctggcccgattgctgggccacgagcacgcctccctggccctggcccagcgctgcagcggcgtgccggcatcgctgcccctgttcaactgcctgctcaactaccgccacagcgctgccgacaccgcgcagcaggggccggcgagctgggaggggatccgcgtgctcgactcccgggagcgcagcaattaccccctggcgctgaatatcgacgatgacgcccagggcctgcgcctgaccgtgcaggccgtggcgcaggtggacggcgatcgggtctgtggctacctgcagtgcgtgctggaacacctcgcccaggccttggagcagaccccggccctggccctggagaacatcccggtactgccggcggccgagcgccgccaggtattgctggagttcaatgccagcacccgcgactacccgcgccagcacaccgtgcaccggctgttcgaggcccaggcccaggcccacccgcagcgggtggcggcggtcgaaggccaggcgcagctcagctacggcgagctcaatacccgggccaaccagttggcccggcacctgctggagctcgggctgcagcccggcgaccgggtggcgatcctgctgccccggtccctggacctgctggtgagccaactggccgtctgcaaatgcgccgcggcctatgtgcccctggatatcaacgccccggcccagcgccaggccttcatggtggaagacagcgccagcgtgctgctgctcagccgcagcgaccaggtgcttgcgtgcccggccagaagggtcgacctggaccgcctgcaactggcgccattgcccgggcacaacccggacctggcgcagtcgtccgagaccgtggcctacatcatgtacacctccggctccaccggcgtgcccaagggcgtgcaggtgccgcaccgggccatcagccgcctggtgctcaacaacggctatgccgatttcaaccccgaggaccgggtggcgttcgcctccaacccggccttcgacgccagcaccatggatgtctggggcgccttgctcaatggcggccgggtgctggtgatcgatcactacaccctgctggaacctgcgcgtttcggccgggccctgagcacggccggggccacggtgctgtttgtcaccacggcgctgttcaaccagtacgtgcaactgattcccgaggccctcaagggcctgcggattctcctctgcggcggcgagcgtgccgacccggcggcctttcgccgcctgctggcgctggcgccgcggctgcgcctggtgcactgctacggccccaccgaaaccaccacctacgccaccacccatgaagtcaccgccctggccgatgacgccgagcacgtgcccatcggccggccgatcggcaacacccgggtgtatgtcctggacgcccagcagcggccgctgcccatcggtgccccgggggagatcgtgatcggcggcgacggcgtggccctgggttatctcaaccgccccgaactcaacgccgagaaatttatccgcgaccctttcagcgaacagcccggggccttgctgtaccgcaccggggacattggccgctggctgggcaacggcctgctcgaatgcctggggcgcaacgacgatcaggtgaagatccgtggcctgcgcatcgagctgggggaaatcgaggcgcgcctcactgcctgcgccggggtcaaggaggccgtggtcctggcccgggaagacgagcccggggacaagcgcctggtggcctactacaccctgcaggcggacgccgcgccgctgccggccgaagccctgcgcgcggccctgcagcagcaactgccggactacatggtgcccctggcctacgtgcagttgcaggccttgcccctgaccaacaacggcaagctggaccgcaaggcgctgccggcgccggccccgtcggccctgctcagccgtgaattcgtcgccccccggggcgaggtggaaatcgccctggcgcggatctggagcgagctgctcaaggtggagcaggtggggcgccacgaccacttcttcgaactgggcgggcactcgctgctggcggtgagcctgatcgagcgcatgcgccagatcggcctgagcgccgatgtgcgggtgctgttcagccagccgaccctggccgccctggccgatgccgtgggcagcggccgggaagtgcaggtgccggccaacctcatcagcgctgattgcccgcgcatcaccccggacctgctgcccctggtggaactggaccaggaggccatcgatcgggtgctggccagcgtgccgggcggcgtggccaatgtgcaggacatctaccccctggcgccgttgcaggagggcattctctatcaccacctgagcgccgggcagggcgacccctacctgctgcaatcgcgcctggccttcgacagcctggagcgcctgcagcgtttcgccggggccctgcagcaggtcatcacgcgccatgacatcctgcgcaccgcggtgctctgggaaggcctgccgcaaccgctgcaagtggtctggcgccatgccgagctccgggtcgaggaacagcacctggacccggccgagggcgacctgctggcgcagttgcagcagcgcttcgacgcccgtcactggcgcctggacctggcccaggcgccgctgatccgcctggtctatgcgcgagagcccgggcagcagcgggtgccggcgatcctgctgttccaccacctggccctggatcacaccgccatggaggtgatcggcgaggaaatgcgcgacctgctgttcgaccgcgcgcagcacctgggcacaccggtgccgtaccgcaactatgtggcccaggcacgcctgggtgccgggcaggcggaacacgaggcgttctttcgcgacatgctcggggacatcgacgagccgaccctggccttcggcgtagaggatgtacagggtgacggcctgggtatcgaagaggccgagctggtgctgcccgaggccctgagccggcgcctgcggcaacaggcgcggcacctgggggtgagcgccgcggccctgatgcacctggcctggggcctggtgctggggcagttggccaaccgtcgccaggtggtgttcggcaccgtgctgatgggccgcatgcagggcggcgcgggcgccgaccgcgccctgggggtgttcatcaataccctgccgctgcgggtcgatgtgcagggcagcgtgcgtgccgcggtcaaggccacccacgcccggctcagcgccttgctcggccatgagcacgcctccctggccctggcccagcgctgcagcgcggtggcggccggcgcgccgctgttcaacaccttgctcaactaccgccacagcgtgccgaacacggcggcgccggacggcctggatatctggcagggcgtcgaactgctggggggcgaggagcgcagcaactacccgctgagcctcagcgtcgatgacctgggcgaaggcttcagcctggcgctgctggcccaggccggaatcggtgcgcagcgtgtcggcgcttatatgcaaagtgccctggagcaactggcccaggccctggaacacaacccgcaggcggccctcgaacagtgctcggtgctgccgccacaagagcgcgagcaactgctggagggctgcaatgccagcaccgccgactacccccgcgggcagaccctgcacgggctgttcgaggcgcgagcggcacaggctcccgacgccgtggcgctggtgcaggggccgttgcgcctgagctaccgccagctcaaccagcaggccaatcagctggcccggcaactgctggagttgggcgtgcacccagacgatcgcgtggccctgtgcctgcaacgcgggccgcacttgttgcggggcatgctggcggtcctcaaggccggggcggcctatgtgcccatcgacccgagcctgccggccgaacgcatcgcctacctgctgcaggacagcgcgccgctggcggtgctggtacaaagcgccacccgcgagctgccgggcagccttgcggtggtgtcgatcgatctcgatggggttgcctggcaggaggccgagctgggcaacccgcttctgccgcacctgaccccggcgcacctggcctatgtgatctacacctccggttccactggcctgcccaagggcgtgatggtggagcaccagagcctggaaaacctggtgcactggcactgcgcgagtttcgacctgggcccggggcggcacagctccagcgtcgccggcctgggcttcgacgccatggcctgggaggtctggccgaccctgtgcagcggcgcgaccctgcacctgccgcccgccgatgtgggcagccaggatatcgaagcgctgctgcactggtggcgcgcccagcccctggatgtcagcttcctgccgaccccggtggccgaatacgccttcagccaggggctcggccaccccacactggacaccttgctgatcggtggcgaccgcctgcgccagttcgccgccgacccgggttttgcagtgatcaacaactacggccccaccgaagccacggtggtggccagctccgggcggatcgaggccggtagcgccctgcatatcggccggcccgtggccaatgcgcggctctacctgctggacgaactgcagcgcccggtgccccagggggtcagtggcgagctgtatgtggccggcgccggggtggcccggggttacctcaaccgcccgcaaatgaccgctgaacgctttctcaacgaccccttcagcgccgagcccgaggcacgcatgtaccgcaccggtgacctggcccgctggcgcgccgacggcaacctcgactacctggggcgcaacgacgaccaggtgaaggtgcgcggcatgcgcatcgaacctggggaaatcgaggcggcgctgctcacccatccggccctcaaggaagccctggtgctggtccgcgaggggcgcctgctggcctatttcacttcccgtaccgagggtgtgcaagccgccgccgaagacctgcgcgaccacctgcaaggccggctgccggactacatgctgccggtggcctatgtgcgcctgccggccatgcccctgaccgccaacggcaagctcgaccgcaaggccctgccaccgccgggggaagaggcctggctgaaccgggaattcgtggcccccgaaggcgaggtggaacaggccctggcgcggatctggagcgaggtgctgcaggtggaggcggtggggcgccatgatcacttcttcgaactgggcgggcattcgctgctggcggtgagcctgatcgaacgcatgcgccagatcggcctggacgccgacgtgcgggtgctgttcggccagccgaccctggctgcgctggcggctgcggtgggcagtggccgcgaggtcgcggtcccggccaaccggatcaccgcggattgcccgcgcatcaccccggacctgctgccgctggtggaactggagcaggaagccatcgaccgagtggtggccagcgtgcccggcggcgtggccaacgtgcaggacatctaccccctggcgccgttgcaggagggcatcctctatcaccacctgagtgcccggcagggcgacccctacctgctgcaatcgcagctggccttcgccagccggcaacgcctggatgatttcgccgcagccctgcagcgggtggtcgagcgccacgacatcctgcgcaccgcggtgctctgggaaggcctgaagcagccgctgcaagtggtctggcgccaggcaggtgtgcaggtgcaagaggtgcaagcgaaccccgcccagggcgaggtcctggagcaactgcaggcgcgcttcgatgcccggcacctgcgcctggacctgacccgggcgccgctgatgcgcctggtgtatgcccaggacccggcccgccagcgcatcgtcgccatcctgctgttccatcacatggccctggaccacaccgccctggaggtggtacgcgaggaaatccaggcctgcctgctggggcagtcgcccgcgggcacggcgattccctatcgcaactacgtggcccaggcgcgcctgggtgtgagccgtgaagagcacgaggcattctttcgcgagatgctcggcgatatcgatgagccgaccctgccgttcggcctgcaggacgtgcagggcgatggcgatgccatcgaagagcgccagcaggccctggagccgtcactgagtgcgcgcctgcggacccaggcccggttgctgggggtgagtgcggcgagcctgttccacctggcctgggcccgggtcctgagcgtcacctcgggccaggaccgggtggtgttcggcaccgtgctgctggggcgcctgagcgccgggcagggcgccgaccgggccctgggcatgttcatcaacaccttgccgctgcgggtcgacctggatgcgcgtggcagccgtgccgcggtcaaggacacccacgcccggctcagtgccttgctgggtcatgaacacgcctccctggccctggcccagcgttgcagcggggtggccgcgccgctgccgctgttcagtgccatgctcaactaccgccacggcagcgacggggtgccgagccaggccgtgcaacaggcctggcagggcatcgagaccctgcacagcgaggagcgcaccaactaccccctgagcctcaacgtcgacgacctgggccaggggttccgtctcaccgccatgaccctggcgcggatcggtgccgagcgtatctgcggctacatgcagcaggccctgctggccctggtggagaacctggagacggcgccgcagcggcccctgcgcgaggtgccgatcctgccgccgcaggagcgtcagcacctgctgcaagggttcaacgccacggccgtggattaccccctggagcagaccctgcacgggctgttcgaggcccaggtacggcgcagccccgaggccactgcggtacaggccggcgagcagcagctgagctacggcgaactcaaccggcgcgccaaccagctggccgggcacctgctgcaactgggcgtcggccccgacagccgggtggcgatctgtgtcgagcgcgggctggaaatggtggtcggcctgctggcgatcctcaaggccggcggcgcctatgtgcccatcgacccgggctaccccgccgaacgcatcgcctacatgctcgacgacagcgcgcccctggcggtgctggcccagggcgcgacccgggcattgctcggcgaactggcgcggcccctggtggacctcgaccagctggcctggagcgggccggcgccgggcaacccgcaagtagaggggctgaccccggggcacctggcctatgtgatctacacctccggttccaccggccagcccaagggcgcgatgaacgagcaccgggccgtggtcaaccgcctgttgtggatgcaggcgcagtaccgccttggcaccgaggacgcggtgttgcagaagaccccgttcagtttcgacgtctcggtctgggagttcttctggccgctgttcaccggcgcgcgcctggtgatggcgcgccccgacgggcacaaggacccggcctacctgcgccaggttatccgcgagcagggcatcagcaccttgcacttcgtgccctcgatgctcgatgtgttcctggcccagggcgagggcgccgaggacctgggcctgcgccaggtgatgtgcagcggcgaggcattgccgggcagcctggtgcggcgcttcaagcagcaactgccacaggtggcattgcacaacctctacggccccacggaggccgcggtggacgtcactgcctgggattgcagcggcccgctggccgataccccggaccacacgcccatcggcaagcccatcgccaatacctgcatctacctgctggacgcgcagatgcagccggtgcccctgggggtggtgggcgagctgtacatcggcggggtgcaggtggcccggggttacctgaaccgcgagcagttgagcgccgaacgtttcctcaaggacccgttcagccaggaaccgggggcgcgcctgtaccgcaccggcgacctcgggcgctatcaggccgacggcactatcgagtacctggggcgcaacgacgaccaggtgaagatccgcggcctgcgcatcgaactgggggagatccaggcgcgcctgacccagctggaggaggtcaaggaggcggtggtactggcccgcgaggatgtgcccggcgaccagcgcctggtggcctactacaccacccacgatagcgcccggcgcctgcccgtcgagcacctgcgcacgcaactgctgcagcacctgccggagttcatggtgccggcgctgttcgtgcacctggcggcgctgcccctgagcgccaacggcaagctggcgcgcaaggaactgccggccccggggctggaggcggcccaggtgcgcgagtacgaggccccggtgggcgacaccgaaatcgccctggcgcggctgtgggccgaactgctcaatgtcgagcgggtggggcgtcacgatcatttctttgaactgggtgggcactcactgctggcggtcagcctgatcagccgcatgcgtgagctaggcatggaagccgatgtccgggcgttgttcgaacagccgaccctggcggcctacgcggccatgaccgaacgaatggagatcgtcctgtgagcgtgatcgaactgttggcgacactgaaggaaaaggatgtgcaactggtgctcaaggacgaccagctggtggtccagggcaacaagcaggccctgagcgaaccgcagctgctggcccgcttgcgcgagcacaagccggagctgatcgagctgatccgcgccggccagtattcgccgagcaaggccgggcaggtgcaggtgccggccaatggcatcaccccgggcatcagccgcatcaccccggcgatgctgcccctggccaacctcgaccaggaggccatcgagcgcattgtcgccagcgtcccgggcggggtcgccaacgtgcaggacatctaccccctggcgcccctgcaggagggcattctctaccaccacgtcagtgcgcagcagggcgacccctatgtgatgcaggcgcagttcgccttcgccagcgaggaacgcctggaagcctttgccgaggccctgcgcggggtcattgcgcggcacgacatcctgcgcaccgcggtgctctgggatggcctggagcagccgatgcaggtggtctggcgcgaggccgggctggagctgcaagaggtcgagaccgaccctgcggccggtgaggttctggcccagttgcatgcgcgtttcgacgcccgccactatcgcctggacgtcagccaggcgccgctgctgcgcctggtgcatgcccgggatgaagccgggcagcgcatcgtcgccatgctgctgttccaccacatggccctggaccacagcgccctggatgtggtgcgccacgagatgcaggccttcctcaccgggcaggccgagcgcctggggccggccatgccgtttcgcaactacgtggcccaggcgcggctgggcatcagcgaacaggagcacgaagcgttcttccgccagatgctcggcgatatcgacgagccgaccctgccttacggcctgcaggatgtgcagggcgatggccgggccatcgaggaatgcacccaggccctgccaaccgagttgagccagcgcctgcggacccgggcccggctggccggggtcagcgcggccagcctgttccacctggcctggggccgggtgctgggcaccctggcgggcaagcacaaggtggtgttcggcactgtgctgatgggccgcctgcaaggcgccgaagccaccgagcgggccctggggatcttcatcaacaccttgccgtttcgcctggatgtggacagccagggcctgggcgaggccctcaaggccacccacgcccggctcaccaccttgctgcgccacgaacacgcggccctggccctggcccagcgttgcagcggcgtgagcgcaccgaccccgctgttcagcgccttgctcaactaccggcacagtgctgccggggccagtgccgcggcccaggccgcctgggccgggatcagcaccctgagctccgaggagcgcaccaactaccccctgaccctgagcgtcgacgacctcgggcaggatttcagcctgaccctgctggccagcacccaggtcgacccgcggcgcatcctcggctacctgctgtgcaccctggagaacctggcccaggccctggagcagacgccgcaactggccctggagcagttgccgatcctgccggtggcggaacgcgagcaggtgctggaaggcttcaaccgcagcgcggtggactaccccccggggcaggctatccatggccgcatcgaggcccaggcgcagcgcaccccagacgccctggcggcctgctaccagggccgctcgctgagctatgccgagctcaaccaacaggccaatgtcctggcccggcaactgcgcgggctgggggtgcagcccgatgaccgggtggccatcgttgcccggcgcagcctggaaacggtggtgggcctattggcgattctcaaggccggggcctgctatgtgcccatcgacccggcccacccggccgagcgtctgaactacctgctccaggactgcggcccgcgggcggtgctgacccaggccgagctgctgggacgcctgccggccctggcggtgccggtgatcgaactcaaccagcggttgtggctcgatcagacggcggacaatacccaggtaccggggctgagcgcggccaacctggcctatgtgatctacacctccggctccaccggcctgcccaagggggtgatggtggagcaccggaccctgggcaacctggtggactggcactgccaggccttcgacctgcgtcccggcagccaggcctcgtgcctggccggcttcggtttcgatgccatggcctgggaggtctggccggcgctgtgtgtgggcgcgaccctgcacctggcaccggcccaggacggcagcgaggacctggatgccttgctcgcctggtggcgtgcccagcccctggacgtgagcttcctgccgaccccggtggccgagtacgccttcagccaggagcagggccacccgagcctgcgcaccctgctgatcggcggcgaccgcctgcgccagttcagccacgaccagggctttgccctgatcaacaactacggccccaccgaagccacggtggtggccagttccgggccgatccacgccggtggcgagctggacatcggccggccggtggccaacgcgcgcatttacctgctggattcccagcagcgcccggtgcccatcggcgtcgccggtgagctgtatgtgggcggcgccggggtggcccgggggtacctcaaccgcccgcaattgaccgccgaacgctttctcgacgatcccttcagcgaccagcctgaggcgcgcatgtaccgcagcggcgacctggcgcgctggctggcggacggacgcatcgactacctggggcgcaacgacgatcaggtgaagatccgcggcgtgcgcatcgagctgggggaaatcgagacccgcctctgccagttccccggtatccaggaagccgtgctgctggcccgcgaggatcagccgggcaacccgcggctggtggcctatttcacccagcagcaggacgtcgccctggacgtggcgcagttgcgcgcccacctgctggcgcagttgcctgactacatggtgccggtggcttatgtacggctcgacgccttgcccctgaccgccaacggcaagctcgaccgcaaggccctgccggcgccggatcaggcggcgctgtttggccgtgaataccaggccccgcaaggcgccaccgaaaccaccctggccgcgatttggcaggaggtgctgcacctgccgcgggtcgggcgccaggatcactttttcgagctgggcggccattcgctgctggccatgcgcatggtgtcccaggtacgccagcgcctgggggtggaactggccctgggcgagctgttcgccaatgccgagctgagcgcggtggccgcggtgctggagcgtgccgggcgcagccatctgccggagatcttcccggcggtgcaggatcacgacctgccgctgtccttcgcccagcagcgcctgtggttcctggcgcagatggacggtgccgccagcgcctacaacattcccatcggcctgggcctgcgcgggcagctcgatcgctcggcgctgcgccaggcgctgcaggccatcgtcagccgccacgccaccttgcgcagccgtttcgtgcgcgtcgaggaccagccccaggtgctgatcgggcccctggacagcgctctggacctgcacgaggaagacctgcgccaggcgccgctgaccctggccgggcgggtgcgtgccgaagccgcccaggccttcgacctggagcaggggccggtgatccgcgcgcgcctgctggccctggcggacgatcaccatgtgctgctcctgaccctgcaccacatcgttgccgatggttggtccatgggcgtgctgacccgggagctggtggccctgtaccaggccttcagccagggccggcccgatcccttggctccgctggcggtgcagtacagcgactttgccctgtggcaacggcgctggctcagcggcgaggtgctgcagcagcagagcgactactggcgccaggccctggccggcgcgcccagcctgctgatgctgcccaccgaccggccgcggccgcagcagcaggatttcagcggtggcagcatcgagctgctgctggaccgggccctgagcgaccagctcaaggccctgagccagcgccatggctgcaccctctacatgaccctgctggccggctgggggctgctgctgagccgcctttcggggcaggacgatctgctgatcggcagcccggtggccaaccgcatgcgcgccgaggtggaagggctgatcgggctcttcgtcaacaccctggccctgcgcctggacctggacccggggcagagcgtgagcgggctgctggcccaggtccgcgcccgcagcctggaggcccaggggcaccaggacctgccgttcgagcaggtggtggaaatcgtccggccccagcgcagcctgtcccatagcccgctgttccaggtggtgctgacctgggtcgacaacttcgcccaggacctgcaactgggggacctgaaactcgaaggtgtggccggggccagcgcggtggccaagttcgacctgaccctgagcctgggagaaagccaggggcagatccgcggaaccctggactacgccaccgcgctgttcgatgaggccacggtgcagcgctatgccgcctacctggtgcaggtgctgcgggccatggtggccgacgatcagcaatgcctggcccaggtgcagtggctggatgagaccgagcgccggcaactgctggaggatttcaacgccagcgccgtggactacccacggggccggaccctggcccagcgcttcgaggcctttgccgctcggcagcccgaggccacggccctgcaggtgggcgcgcagcgcctgagctatggccagttgaatgcccgggccaaccaactggcctggcacttgcgggagctgggggtgggcccggatcagcgggtggcgatctgcgtcgagcgcgggccggggatggtgatcggcctgctggggattctcaaggccggtggcgcctatgtgcccatcgacccgggtcaccccgcggaacgcatcgcctacctgctgcaggacagcgcgccgctggcgctgctggtgcagggcagcacccgggccctggtgggccaaccggccatggcccgggtggacctcgaccagcccgagtggcaggcccgccccgagggcaacctgcaggtgccgggcatgagctgcgcgcacctggcctatgtgatctacacctccggttccaccggcctgcccaagggcgtgatggtggagcatgccagcctggaaaacctgctggactggcattgccaggccttcgacctggggccggggcgacatgcctccagcgtcgccggtttcggcttcgacgccatggcctgggaactctggccgacgctctgcggcggcgccaccctgcacctgccgccggccggtatcggccaccaggacctcgaccaactgctgcactggtggcaggcccagcccctggacctgagctttctgccgaccccggtggccgaatacgccttcagccagggcctgggccacccgaccttgcgcaccctgctgatcggcggcgaccgcctgcgccagttcagccaggagccgagctttgccgtggtcaacaactacggccccaccgaaaccacggtggtggccagctccgggcggctgctggccggcgggcccctggacatcggccggcccatcgccaatgcacggatctacctgctggacccgcagcagcggccggtgcccatcggcgtcgccggtgagctgtatgtgggcggcgccggggtggcccggggttacctcaaccgcccggagctgacggccgaacgcttccttgaagaccccttcagcaccgtgccgggcgcacgcatgtaccgcagcggcgacctggcgcgctggcgggcggacgggcgcatcgactacctggggcgcaacgacgaccaggtgaaggtgcgtggcgtgcgcatcgagctgggggagatcgagacctgcctcaaccgccttccgggcatccgcgaggcggtgctgctggcccgggaagaccagccggggcagacccggctggtggcctatttcagtgcaacccccggcgccgagccgagcccggccgagctgcgcaggcgcctgctggagcaactgccggagtacatggtgccggcggcctatgtgcacctcgctgccttgcccctgaccgccaacggcaagctcgaccgccgggcgttgccgcaaccgccgctggaggcgctgctgagccgggactatgtggcccccgagggcgacagcgagatcgccctggcgcagatctggagcgagctgctgcaggtcgagcgggtagggcgccacgatcacttcttcgaactgggcggccattcgctgctggccatgcgcatgctgtcccaggtgcggcagcgcctgggggtggagctggccctgggcgagctgttcgccaatgccgaactgtgcgccgtggccgcggccctggcccgggccgagcgcagcagcctgccggacctgctgccggcgccgcgcaaccagccgctggcgctgtccttcgcccagcagcgcctgtggttcctggcgcagatggaaggggccaacaccgcctacaacattcccatcggcctgcgcctgcgcgggccactggacggcgcggccctgcagcaggccctggagcgcatcgtcgcgcgccatgaaaccctgcgcagccgctttgcccagtacggtgacgaagcccaggtgctgatcgccccggccgaatccgggctggagctgctgctggaaaacctgcggggccacccccaggccagcgacgccttgcaggccctggtgcagggcgaggcctcggcgccgttcgacctgcaacgcgggccgctgatccgcgggcgcctggtcagcctggcggacgaccaccatgtgctgttgctgaccctgcaccatatcgtcagcgacggctggtccatgggcgtgctgacccgggaactggtggcgctgtaccaggccttcagtcgtggcctgcccgaccccctggcgccgctggcggtgcagtacagcgactttgcccactggcagcggcactggctcagtggcgcggtgctggagcagcaggcgcagtactggcgccaggccctggagggggcgccgccgttgctcatgctgcccaccgaccggccacggccggcccagcaggactacgccggcagcagcgtcgaggtgcgcctggatgcgcaattgaccgccggcctgcgggccctgagccagcgccatggcaccaccttgtacatgaccctgatgggcgcctgggcgctgctgctggggcggctctcggggcagtccgaggtggtgatcggcaccccggtggccaaccgcatgcgcgccgaggtggaagggttgatcgggctgttcgtcaacaccctggccctgcgcctggacctggaggcggcgccccgggtccaggcgctgctggcccaggtccgcgcccgcaccctggaggcccaggctcaccagcacctgccgttcgagcaggtggtggagatcctgcggccgttgcgcagcctgtcccacagcccgctgttccagaccctgttcacctggcagaacggcgacggcccgcagctggagctgggggacctgcaactggagggcatccacgaggccagccacttcgccaagttcgacctgtccctgagcctgggggaggtgcaggaccgcatcgaaggcagcctggaatacgccacggcgctgttcgatgaaaccaccatgctgcggtatgccggctacctgcagcgggtgctgcaggccatggtggccgatgaacagatgctgctggcacaggtgccgctgctggatgccgccgagcgcgaacagctgctgcacggcttcaatgccaccgcccgcgactaccccctggaacagaccctgcacgggctgttcgagacccaggtgctgcgcagccccgaggccatcgccgtgcaggccggcgagcagcagctgagctaccgcgagctcaaccagcaggccaaccagctggccgggcacctgctgcacctgggcgtcggccccgacagccgggtggcgatctgcgtcgagcgcgggctgcccatggtggtgggcctgctgggcatcctcaaggccggcggcgcctacgtgcccatcgacccgggctaccccgccgaacgcattgcctacatgctcgacgacagcgcgccccaggcgctgctggccgatagcgcgacccggccgctgctgggcaacctggcgctgcccctggtggatctcgaccagccgcaatggcaccgccagccccgggtcaacccgcggctcaacggcctgaccccgagccacctggcctatgtgatctacacctccggttccaccggccggcccaagggcgcgatgaacgagcaccgggccgtggtcaaccgcctgctgtggatgcaggagcagtaccgtctcactgccgaggacgcggtattgcagaagaccccgttcagtttcgacgtctcggtctgggagttcttctggccgttgttcaccggcgcacggctggtgatggcgcgccccgatgggcacaaggacccggcctacctgcgccaggtgatccgtgatgaacgcatcagcaccttgcacttcgtgccctcgatgctcgatgtattcctggcctatggcgacacccgcgaatgcgccggcctgcgccaggtgatgtgcagcggcgaggcgctgccgggcagcctggtgcggcgcttcaagcagcagttgccgcaggtggcgctgcacaacctctacggccccacggaagcggcggtggacgtcaccgcctgggattgcgccggcccgctggcacagaccccggacaacacccccatcggcaagcccatcgccaatacccgcatctacctgctggacgcgcagatgcagccggtgccccggggcgtggtgggcgagctgtacatcggtggggtgcaggtggcccggggttacctgaaccgcgaacaactgagcgccgaacgcttcctcaaggacccgttcagccaggaaccgggcgcgcgcctgtaccgcaccggcgacctggcccgctacctggccgacggcactatcgagtacctggggcgcaacgacgaccaggtgaagatccgtggcctgcgcatcgagctgggggaaatccaggcgcgcctgacccagctcgaaggggtcaaggaagcggtggtgctggcccgggaagatgtgccgggcgaccagcgcctggtggcctactacaccaccgttgccgggcagccggccctggcggtggaacaactgcgccgggcgctgctggaacacttgccggagttcatggtgccggcgctgttcatgcacctggcggcgctgcccctgagccccaatggcaagctggaacgcaaggccctgccggccccggggctggaggcggcccaggtgcgcgaatacgaggccccggtgggcgacaccgagatcctcctggcgcaactctgggccgaactgctcaaggtcgagcgggtggggcgccacgaccacttcttcgagctgggcggccattcgctgctggcggtgagcctgatcggccgcatgcgccgggccgggctctcggcggatgtgcgggtgctgttcggccagccgaccctggcggccctggccgctgccgtgggccgcggtcgcgaggtgcaggtaccggccaacctgatctaccgcgactgcccgcgcatcaccccggacctgctgccgctgctggcactggaccaggccgccatcgaccgggtggtggccacggtgccgggcggtaccgccaatgtgcaggacatctacccactggcgccgttgcaggccggcatcctgttccaccatctggcggctggagcgggtgacccctacgtgttgcaggcgcagttcgcctttgccagcaccgagcgcctgcaggccttcgcccaggccttgcagggggtgatcgaacgcaatgacatcctgcgcagcgcggtgctctgggaaggcctggagcagccgctgcaagtggtctggcgccaggcgccgctgtgctgcgaagagatcgccctggagcctggcgacggcgaggtgctgggccagttgcaggcgcgcttcgacagccgtcgctaccgcctggacatcgcccaggcgccgctgctgcgcctggtgcatgccgccgacccggtgaaccagcgagtggtggccctgctgctgttccatcacctggtgatggaccacgtggccctggaggtgctgcagcacgaactgcaagccttcctcctgggccagcagcagcgcctgggggaagcggtgccgtaccgcaactacgtggcccaggcgcgcctgggcatcggcgaggccgagcatgaggcgttcttccgccagatgctcggcgatatcgaccagccgaccctgccgctgggcctgcaggaagtaccgggaggcagcgccgaactcagcgaagcgcgccagcccctggaccgcctgctgagccggcgcctgcgcttgcaggcgcggcaactgggggtcagtgccgcaagcctgatgcacctggcctggggccgggtgctgggcagcctggcggggcagcagcaggtggtgttcggcaccgtgctgctggggcggatgcagggcggcgagggcgccgaacgggccctgggagtgttcatcaacaccttgccgctgcgggtcgatctgggcgagttgccggtgcgcgaggccctgctggccacccatgagcggctggcgcagctgcttggccacgaacaggcgcctctggccctggtccagcgttgcagtggggtggagccgggtacaccgttgttcagcagcctgctcaactaccgccacagtgccccggcggcaaaccaggcggggcaggccgacagtgcctgggagggcatgcagttgctcaatgcccaggagcgcagcaactacccgctgaccctgagcatcgacgacctgggggacggcttcatgctcaccgccgtggccgccgggatcgatgcccggcgcatttgcgactacctgcacggcaccctggaacagctgctgctggccctggagcagcaaccggaacaggccatcggccaggtgccggtgctgccgcaggccgagcgccggcaggtactggagggcttcaacgacacggtgcgcgactacccgcgcgagcaggtcctgcaccagtgtttcgaggagcaggtgctggcccggccgcagcaggtggcagcggtgcagggcgccgaacagctcagctatatccagctcaacacccgggccaaccaactggcccagcatctgctgcaactgggggtgcagcccggggatcatgtggcgctgctgctgccgcgctccctggacctgctggtgagccaactggcggtgagcaagtgcggcgccgcctatgtaccgctggacgtcaatgccccggccgagcgcctggggttcatgctggccgacagcggggcgccggtgctgctcagccacagcgagcgggtgctggaagctgcggtgcaacgggtcgacctggaccgtctgcgcttcgaccggctggccgggcacaacccgaacctggcgttgtcgtccgaggcggtggcctacgtcatgtacacctccggttccactggagcgcccaagggcgtgcgggtgccgcaccgggccatcacccggctggtgatcaacaacggctatgccgatttcaaccccgaggaccgggtggccttcgcctccaacccggccttcgacgccagcaccctggaggtctgggggccgctgctcaacggtggccgggtcgtggtggtggaccacgcgacattgctggacccgcacgccttcggctcgctgctggaacgcaccggggtcagcctgctgttcctcaccaccagcctgttcaaccagtacgtgcaactggttccccaggccttcaaggggctgcgcatgctgctctgtggcggcgaacgggcggatgccacggccttccggcggatccaggccgagctgccgcacttgcgcctggtcaacggctacgggcccaccgagaccaccacctttgccgtgacccatgagcccggtgagctggcggcggatgccgacagcgtgcccattggccggccgctgtccaacacccgggtgtacgtgctcgatgccctgggccagccgctgccggtgggggtggtgggggagctgtacatcgggggtgatggcgtggccctgggctacctcaaccgtccggacctgtcggcgcagaagttcctcatcgacccgtttcaccagcaaacccagggcaccgctgcgccggccctgatgtaccgcaccggcgacctgggccgctggctggaaaatggtcttctggaatgcatcgggcgcaatgacgagcaagtgaagatccgcggcctgcgcatcgaacccggggaaatccaggcgcagttggcccgcttccccggcctgcgggacggcgcggtcgtggtgcgtgaagacagccccggggacaagcgcctagtggcctactacaccctgcacgagcacaccccggcgccggatgccgagcaactgcgcagccacctgcaacagcagttgccggactacatggtgcccctggcctatgtgcacttgcaggccttgcccctgacccgcaacggcaagctggaccgccgtgcgctgccggcgcccggcagcgcggcccagctcgaccaggcctatgccgcgccccagggcgcgctggagcaggccctggccgggcattgggcggcggtgctgaagctcgagcaagtagggcgccacgaccacttcttcgaacagggcgggcattcgctctcggccatccagttgctcaaccgcctgcaacaggcggcgttcgacgtgaccctggcggagctgttccagcacgccacggtggcggccatggcgcgcctgttgagcgagcgcacggccgcgccgcgctccaccgagctgatcccggtgcgcaccggcggcagtggcccggcgctgttcctggtccatgagttcaccgggctggacgtgtatttcccggccctgggccagcacctggaaggcgatttcccgatctacggcctgcccggcgtggcggtcggcgagccgcagttgcgcaccctggaatgcctggccacacgcttgcttgacgtgatgcgcaaggcccagccgcaggggccgtatcgcctggccggctggtccttcggtggggtcctggcctatgagatcgcccagcagttgctgggcctggaccaggaggtggagttcctcggcctgatcgacagctacgtgccgcgcctgaccgaccagggcaaggctcgctggtctggcgagcacgcgcacaagcgccacctgctgttgcagtgccgcgcctactggagcgcccagggcgcggccggggtgcagcccctggcgcacctggaacaactggaggcgctgctggagccactggactttgccgacctgctgcaacgctgccgcgaccaggggctgctgttcgagcaactggccacggcgccgccgcaagcgctcggccattacctggaccgcgaggtcgcccacggccatgccctggcccattaccgggtccatcccctgggcatgccggtgcatctgttctgcgccgccgaacggcccaccgagctgtcccggcgcagcccgaccctgggctggggcgagatcctgccggcgggcgaactgcactgcatcagcgtgcccggggaccacctgagcatgatgaaggcgccgcatatccaggccctgggtcacgccctgggccaggccctggcggcggccagggccaagggcgcggtgagcgctgtgccgcaccagcccttgctgcggatccagagcgggcgccccgggcacacgccgatcttctgcgtgcccggggccggtgacagcatcaccggctttatcggcctgagcgaggccctgggggccgaatggccgatcctcggcctgcaggcccggggcctggacggctgcggggtgccccatggccaggtcgaagtggcggcccggcattacctggaggccatcaccgccgagtaccccgacgggccgctgcacctgatcggccattccttcggcggctgggtggcattcgagatggccggggccctgcaggccgccggtcgcgaagtggtgtcgctgaccctgatcgacagtgaggctcccggaggcaatggcgcgctagggcggccctataccgccaccgcggcgctgtggcgcttgatcgaatccatgcaactggctgcaggccaggacatgggcatcgaccccgagcgcttcgccgccgaggacgacggcatgcagatgcacctgctgcatgccggcatggtgcgggtcgggctgttgtccccgcgcgccgacccgcgggccatgcacggcccggcgcgaaccttcgccagcgccctgcgtacccgctaccagcctcggcgcccgtaccagggcaaggtgcgcctggtgctggcggatgacccgagcctggacgccgccggcaaccgccgggagcagcaggggatggtccagggctggcgccggcaggctgcgcacctgcaggtgtggtacggcccgggcaaccacttcagcctgctcaaggcgcccaacgtctaccacctggcggcctggtggcatgacggcctggcgctgccccagggggaggtcatgtcatagggcgcggggcaacacaggcgctggccctgccggcgcctggacacccggtttcattcatctggaacaccgctcactacggcagcacggtgttctgtaagggggctcgccgctggccggtgcagcccgccctcaacggacttgtggtcatctatggaaaagtcgaagtttcgcaaaatcggtatggggctgttgctggtggttgtggccgggttgattttctatacggtgcaggcgccggcggagccgccgcagtacctgaccgccaaggtcgaacggggggatatcgagaacgcggtcctggcctcgggcctgctcgaaggcatcaagcaggtggatgtcggggcccaggtctccgggcagttgaagtcgctcaaggtcaaggtcggggacaaggtgaaaaagggccagtggctggcggaaatcgacccgctggtgctgcagaacaccctgcgccaggcccaggtggacgaggagaacctgcaggccaagcgccgcgccaccgcggcccagctcaaggaaaccaaggccatctacgagcgctaccgtgacttgcagtccgacgcctcgatttcccagcaggattacgagaccgccgaatccaactacgaggtgcagcgggccaacctgctgtccctggacgcgcagatcaagagcgcgcacatccagatcgacaccgccaaggtcaacctggcctacacccgcatcgtcgcgccgatcgatggcgatgtggtgggggtggtgacccaggaaggccagacggtgatcgccaaccagctggcgccggtgctgctcaaactggcggacctggacaccatgaccgtcaaggcccaggtttcggaggccgatgtgatccatatcgcccccggccaacaggtgtacttcaccatcctcggcgaggcggaaaaacgctactacgccaaactgcggggcactgagccggcgccgcagaactttctcgaaacccagaccgccggcacccccaagcagaacaccgcggtgttctacaacgcgctgttcgacgtgcccaacccggaccatcggctgcgcatttccatgactgcccaggtgcgcatcgtgctggataccgccaaggacgtgctgatggtaccggtggcggccctgggcccgcgcaatgccgacggcagcttcgcggtgcgggtgctggacgccaagggccatgcccaggcgcgcaacgtcagcaccgggatcaacaacaacgtcaaggtgcagatcaaggacggcctggcggagggtgaccgggtggtgattggtgatccgctgcccggcacggcaggggcctgagcatgagcgagcccctgctgcacctcaccggcatcagccgcagcttcaccgccggggatcgggagttcctggccctcaagcacatcgacctgagcatccaggccggggaaatggtggcgatcaccggggcctcggggtcgggcaagtcgaccctgatgaacatcctcggctgcctggactacgccaccgccggcagctacaaggtcaacggccgggaaacccgcgacctcgacgaccaggccctggcggaactgcgccgcgactacttcggcttcatcttccagcgctaccacttgctgccgcacctgagcgccatgcacaacgtcgagatgccggcgatctacgccggtaccccgcaagtcaggcgccatggccgggcccgggaactgctggcgcgcctgggtttgtccgggcacctgggccaccggcccagccagctctccggcggccagcagcagcgagtgagcatcgcccgggcgctgatgaacggcggcgaagtgatactcgccgacgaacccaccggtgccctggacaccgccagcggcaaggaggtgatgaacatcctccaggaactgcacggcctggggcacacggtgatcatcgtcacccacgaccccaaggtggcggccaacgcccagcgcatcatcgaagtgcgcgacggcgagatcgtcagcgaccgggccaacccgcgcccggcggacgaggcgccgagcgagccaccggtcagcgtgcggcccgccggtgcgcggcgcctggtggccagcctggggctgttcaaggaggccttcgtgatggcctgggtggcgctggtctcgcaccgtatgcgcaccctgctgaccatgctcgggatcgtcatcggcatcacctcggtggtgtccatcgtggccatcggcgagggcgccaagcgctacgtgctcaaggacatccaggccattggcagcaacaccatcgacatcttccccggggccagtttcggcgacagccgggcggcggcgatccagaccctgatgcccgccgacgtgacggccctgaaccagctgtactacgtcgacagtgccacgcccatggtcggccgcagcctgttgctgcgctacggcaacatcgacctcaacgccacggtcaacggcgtcagccacctgtatttccaggttcgcgacatcaagctggccagcggcatttccttcagcgagaacgacgcgcggcgccaggcccaggtggtggtgatcgaccacaacacccgcaaccgcctgttcgggccggatgtcgacccgctggggcaggtgatcctggtgggcaacctgccgtgcacggtgatcggcgtgacccgggagaacaagaacatgttcgccgccagcaacctgctgaacgtctggctgccctacgagaccgccgccgggcgggtgctcggccagcgccacctggacagcatcagcgtgcggatcaaggacggccagccgagcaaagtggtggaggagcatgtgaagaagctgatggagcagcgccacggcaccaaggatttcttcaccaacaacctcgacagcatcatgcagacggtgcagcgcaccagccgctccctggccctgctgctgtcgctgatcgcggtgatttccctggtggtggggggaatcggggtgatgaacatcatgctggtgtcggtcaccgagaggacccgggagatcggcatccgcatggcggtgggggcccggcagtcggatatccgccagcagttcctggtggaggcggtgatggtctgcctgatcggcggggccatcggcatcagcctgtcgttcgccatcggctacctgttcaccctgttcatcaaggagtgggagatggtgttttccatggggtcgatcattaccgcgttcgcctgttcgaccctgatcggcatcgtcttcggcttcgttccagcgcgcaacgccgcgcgcctggatcccatcgaggccctggcccgggattgacaggccgtagaccgaaccgccggcgctggggccggcgggggttacagggccaaggccggatcaggccgtcggggtttcgctgctgtgcggcgcttgttcctcgggcgagtacatccagcgcagcagcgagtgccggccactgatgccgagcttgatggcggcgcgcttcaagtagctttcgacggtattgaccttgagcttgagctgctcggccagctgcggcgcggtgcgcccggccagcagcccgacacagacctccagctcgcggttggacaggcgcagcccggactgttccaagcgcgcggcaaagcgcaggcgcaggtttgccaggccctcgatggcggcactgtcctgctcggcggcatcgccccggggctgcagggcgctgatgtgtttttccaccatgggcagcagcaacggcgagatgtcctccagcagcaggcgttcctggggggaaaagctctggtcctggctgctgcggtacaccgagagcacgtagtggtaggagtccttgcgcccggtcaggtgcagttgtgccgggtccgcgtggccgcgctggcgttgcagcgacgggcccggggccggttcgccgggttgttcgggaaagccggtgagcagcagcgggtcattgctggcatgggcatcgctgtacagcgggtggcgggtctgcatcagggtgttgccggttctgccgatggcctgttccgggtcggcccgcagttgggtgatatgggtggcgtccacagccagctgggtgagtatcaggtcgtgcagcatgcgcggaaagttgcggctgccggtgctggcgatgaccttaccaatgtgtggaaacagtatttgtgagttcatcatttcatccatgaatgtgaaggtgtggaggggggctgccttatagcaacgagtttgggtcagggttgatttacctcctgtttgctctgggcgacaaagggtcgcctcgtatcctagtacagggttgcggtggctgtttgatgaagtgcgcaaaatctttcccggcgggcgcagcggtgcggtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccccggngttgcnnnncntgcngcgttggcttgcaggtgggccgggttacgggcgcttctcgcaattgaccatccatggcacaccaaagcggtccaccagcatgccgaagcgctcggcccagaaggtggcttccaggggcatctgcacgttgccctgttcggccagggcggcgaatacccgttcggcttcggcggcgttgtcgatgttcagcgagatcgaacagccgtggatgcccaggtagggcagctcaggggtggtgtccgagcccatcagcacctggtcgccgacgctcaggcgggtgtgcatgatgcgttcgtgacagtcggcagagatgtgatcacgggccgggctttcaccgaaggtcatcatcatttccagggtgccgttcaggcattgggcatagaaggtgaaggcggccttgcaggtgccgttgaacaccaggtaggggttgattttcatgctgtgctccggtcgcaggtaacgggcaggcacgcgggcctgtggacgaacttccatagcaaagccgctatcggccctgcgcggaggtttttttatagatggtttttccatgctcgcccgtggcgatataaccgtcgccgcgccattgggggcgcggcggcggtgcgggcgggggttacaggcgcggctgctcctggggcgtgccgagcaactggtgcagggtgttgatcagcgcctgctggcgcagccggtcttcctggtgccgggcttcggcggcggcaacatcggcgttgcagtgcgggcaggtgctttcatgggcgtggatgtactccaggcaactgcgacacagggccaggcgcggcgggatgcggccttcctcgggcgacgcctggccctggttgacccaggccagcttgatcacctggccgctgtcgttgacgctcgataccgcctcgccggtgtcgatgcgttcggtgatcaggtcgaaacggcccaccgagaacgacgccgggccggcgtcttcgagcttgacgatgcgttcgcgcaggccctttttcgccaggtccagggtccggtagtggcagtagggattgttccccggcttgcccagcagagagtgcgaggtccaggtgcagccgccgcggcagacatcgttgtagtaacaggtgcggcagtagccccacaggtcgtccaccgagcgcaggcggccgaagtggatgccttcgctgtagtgccagatgtcgtgcaggctcatgctgcgcacgttgccaccggagaaacccacggtggccagggacgggcagcccttgaccgtgccgtcggcctccagggccaggaccgtctgcccggcggcgcagccgctccagtgcacgcggtcgtcgccgaagccgcgccacatgtgctcgtaggggccgtagtagccgatgttgttgcccacattcatcagcagcccgcgctcggcgccttcgcggtacaggcgcgccagcagcggcatcacctccagcaactggtagggctgcagcagcagctcggggtgatccaccgcattgcccatggccacggtcagctggatctgccagtggctggcgccggcatcgatgatcagttccatcagggccggcaggtcgggcagggtggcggcgccgatctgcgtgttgacgctgaccttgagcccggcctgcttggcccggcgcagggtatccagggccttgtcgaaggagccggggacattgcgcaccgcgtcatgcagcggcgccaggccatccagagagacgccgacgccattgagcccggcatccaccgcggcctgcatcttggccggggtcaggttgcgcccgccggtctggatcgccacgtacatgccgtggtcgtggatggcctggatcagccgcgtccagtccttgcgcagataggcttcgccaccgatcagggtgacttcccgggtgcccagggcggcgatcgaatcgattaccgacaggcattcctgggtattgagttcgcccgggcgccgatgcccggcgcgggagccgcaatgcaggcacttgaggtcgcaggcgagggtgatttcccagaccacatgcaccgggacaaagcgcttgaggtcggtttcgctgaggtagcgggcggggagacggtctgacatgagagttccttgcgcacccggcggtgcgcggcaccaacccatcgaaataggcgcgccttccttgtgtcgcaatggcgcggtcgttttgtggcagcccccggggccgcggcgggcggccccgggtgctggtggtcagcgctgttccaggcggctgatttctgcctgcaactgctgcaccgcctgggtcaggttgtcagcgttggccagttgctgctcgccctgggccaccagtgccttgagccgcgccaggtcgccgctggcctgggcctgctgcagcgccacggcatacagcgggtggcgcacttcggcggccgggccggcctggctggcgtagtccgctgcctgcgcctgctccaccggggccgggctcacggcggcgtgttgcaccacgtgccagtgcccctggaactggtacttgtagctggcgaagccgctggcccagtccaggccgaggatgccctgcaggtggaaggtctcggcgatctggctggtggggccgctggggttgccggccaggctgaggatgatgtgcccctcgctggaggggtcgagcttgacctgcgagtagtcgccccacacgtcggcatggaactgcaccggtggcgaggtgctctgggtcacgcgggcaacgccgctgacgcgtttcttcggggtatcgaccagcaggtcgagggtcagcaccggcccaccgatcacggggttggcgacgatgaggcgggtatggaacagtccgattgtcatgctgcaactccttatcttcttgaagacatcaggttcgatggacggccgggctcagcgccgctcctgctggctgatctcggccttggcggccaccagtgcggtgcggatcagcggcagctgttccagttgctgggtggcgaccttgtgcaggctcttcatctgcgccaggtcaccgctggcgatggcaccatggatcggcgccccgtacagcgccgagatcggcggcagcggatggttgggctggccgcggtgcagaggggccgacagggtgctggactgcacggtttcctgcaggtgggcgggcacctggttgaccgtcacccagctgtggccgttgtagtactgatagttggccacgccttccttccagtcgtcgctgaccaccaggtgcagcttgaagttgatcaccgagttcgaccccgggccgccctggttgccctgggccgtgatgaggatcttgctggtgctgggcggcatgacggtgaggtaggtgtactggccccagacatcgctgtgcaggtccaggggcgggttgaccgcctgggtgatgctggaagtgccgcgcacactgtgttccggggtataggccagcaggttgaggatcagggtgggtgcgccgggttgcgaggtgccgatgcggtagctcagggggaacaggccttcggcttgcgagtagttgccggacatttgctttgcctccattcaatgacaggggaaccgcgatcagcggcgttccaggcgggcgacttcctgggccagctgttcgtaggcgttgtgcagatccttgggttcgttcctggccggatcgcgcttggccagcagggctttcatctccggcagcttgccttcgctgatggccgactggatggcgactccgtagggggggatcacatgttgggatgcattgctcatggcgtttgccttccgtgggtgtggatggggtgctgcgctactcggcagcttttgcagttaagcagcgcgggtggaattggcagggtaatggcacattgattttgtgggtttccgaccggtggattcacggtcggtaaagggttgattttggagacattccaaagggcgatatgcaggcgggaggtgattgcggatggcgataggagggggtgagaaaggggggcgagcgtaggcaccggctgccggcgaagaggcccgcgacagccatgcatcgccctggccggcgccttcgctggcaagccagcccctacggggtgtcagtgcgctcggaccacccccaactcctccagccggcgccgggtctcagccgccgtcacatgatgaatcgcatgccaccccagcgccgttgcggcgacgatattgggcgcatggtcgtcaatgaacaccagttcctcgggggccacccctggcaaatgcgcacgaatgcgccccagggtcgtgtgatagatctcggcatccggcttgatctggcgctcttgccccgacacgacgatgtcgcgaaagcgctgcagaaacgggtaattgtcccaggcatagggaaaggtttcccgggaccagttggtcagcccgaacaggggcacctgcgcctggtgcagttcttccagcagggctacgccctgatccagtggccccttgagcatctccggccagcgcccgtaataggcctcgatcaagggggcatggtcggggtgctcggccaccaggctgcgggtcgcctcggccaggctgcggccggcgtcctgccgggtattccaggcctgggtgcagatggtgtcgaggaaccactggcggcgctcatcgtcggggatcagctggcggtacaggtggtgcgggctccagtcgaacagcacgccaccaaaatcgaaaaccacggcacgaatcgtcatgaacacgtccttttgagcggttatgggccaggcgccatcctgcgacgcccggcagcacaatgcaatttgccgctggccgctggtccgctcgccggcaccctggccacgaatcgtgatccaacccctgtatccggccttttccaggctggcacaggagctgcccatgactgccaagaaccccacggggccgcgctcgtccgagcatgcctcgaatcatcatgacccgggtttcgaccccgattcgccagacctggccgacccccaggtagacccggtaggcccggccagggcacccctggaccgccctgaacccgagcaccggcgcaatcgctcgcgggcctacgatcccctggccaacctgaagaagtgacccggcgcgccgacatgcggcagtcgccgggcggtgtggcgggtgtaaggtaaaacgcccaagccccaggctcgccgaggaaaaaggaggtgtccatgagcacgccgctgctgaacaaactgcagatcaacggctatcaggtcttgagcgtcaaccacgggccctggaaggtctgcacgccgggtgaccggcttgctaccttcggcacccgggaagaggccatggcctttgccgccgccctgccggcgcgcaggcaacgaccggccgccaccggtggctagcgcccaggcaggaaaaaagccccgaccggttcggggcttttttatgcctggccgggggcatggccggacggggcgagggcgcagcggggcgcaattacggctgcatgaaactcagcagcgggaggatgaaggctgctgccagggccagtgcccacagggtctgcagaaacagctcatcggcgttgtcggcggcgggccgcggggcgaggcggcggcgccagtagaagtacaggccgctgaccacgaacacacaggtgatgcccatcatgtactccagctcccacttctgggggctgatggccaggctgccgtcctggtcatccgggtagtcgagccgccaccaggcgcccagcagcagcggcaccagtccggccaggagccaggccaggcgcgcgccgggcatccgggtcagcagggtcacggccaggccgatcaggcacagcacccagacaaagaggtacagctgaaagcccatggccccaccgaacagcgtgctgctgacatcccgcggctgcatcatctgcacgtcgcacagcagcatcagcgacaagggcgcggcaacgacgatccagcacagagggcggtagagccacaaccacagggtaaacaggcgagacatgggcacactcatcgaggtttggtgaagcaagaaagaggcggggttgacggtcagtcgacaatgacccgggtgccagggcccaggcacaggctcaactgttcgccggcccggttgaaggcgcagtacggcaggggcgccttgctggcattgaccaagggggggatgaagaagccccccagcaacaccgcccatgcaatccgcaacgacagctcacggagggtggcgggggctctcggcttgagccgctgctggtgaatcaggtagatgccgctgtagaccagggcgaaaccgaagaccatcacatagtccagctcccgggcctggatgctgaagatcaggttgccgtcgaaatcatccgggtagtggatccgccaccacaggcccagcagcaactgcaccagcccaccgatgatccaggccaggcgggcgccgggcacacgggtcagcacggccaggcccaggccgatgacgctgaaggcccagccgcccaggtacagcatgaaacccatggagccgccgaacagcgtgccgccgacttcccgggggacgatcaggtgcaggttgcaacggatcatggtggccaattgtgcggccacggcgatccagcacaggaggtagtagagccgggaccagaaggtgtgcaggggcgacatgcagagactcgaaaaaaacagggccgatgagagtgtgtggtgggctgggttcaaaccaacggcaaaccgcaggcaaccgcgaacagcgccagggcgatgctgatgcccgccaggggaatgcgaaaccagtaacggatggccagcacccacaggctggcgagaaaccccaggccgatcagcagcaagccacgggcctccagcagcagggccgggtgcagccaggcgaggtagccatagaccaggccgaacagcatggcgcccaggctgtggctggcgttgaagccgacccaggcccgccacaggctggtctggcgggtgatcatcggtgacacctggcgcatgcgttcggccagggccgggtcccggggctggaatttgtcggtggcgaaggtgtagatcaggtgcagggtgccgagcagcagcacgatggctgagctggcgatgatcagggcgggggccaacacgtttcttccttgggcggtgctcgggtgcgcggagtatgccgggccgcccggggttttgcacctgcgggcggtggttttttgccagcacggggcagggcgccgggcgccctgcccaggggctcagaagtgctcggcgtcgaggccgaacaaggaggcgctgccggcgcggatgctctgctccaggctgaggatgcgcggcagcatgcggctgaagtagaagcgcgcggtggccagcttggcaccgtagaaaccgtcgtcctcggaacgcttgtgctcggccacctgggccatgcgcgcccacagataggcataggccacgtagccgaacagttgcaggtattcgacgcaggcgctgcccaccgcgtgggggttctgcgccgcctgttcacgcagccagtcgctgaggtcttccaggcgctgcaccgcatccagcagctcggcggcgtaggcggcgccgggcaggtaggcgtagtcgcggatctcgccggtgaacaggcgcagggccacgccgccgttgcccaccaccttgcgccccaacaggtccagggcctggatgccgttggtgccctcgtagatctgggcgatgcgcacatcccgcaccagttgctcctggccccattcgcggatgtagccgtggccgccgaagatctgctggcccagcacgcaactgtccaggccggtgtcggtgaagaacgccttggccaccggggtcagcagcgccaccagggcctcggcgttgtgccgctcatggggctggtcggcgaacttggccaggtccagctgctggccgacgtagctggcgaaggcgcggccgccttcggtcatggccttcatggtcagcagcatgcgccgcacatcggggtggacgatgatcgggtcggcgatcttgtccttggccaccgggccggccggggagcggctctggatgcgctcgcgggcgtaggttcgggcgttctggtaggagttctcggcgcagccgataccctggatgccgatggacaggcgctcgtagttcatcatggtgaacatcgccgccaggcccttgttggcctcgccgatcagccagccgctggcgccgtcgaagttcagcacgcaggtggccgaggccttgatgcccatcttgtgctcgatggaaccgcagctcaccgcgttggccgcgccgagattgccgttggcgtccacatggatcttcggcaccaggaacagcgagatgcccttgggcccggccggggcgtccggcaacttggccagcaccaggtggatgatgttctcggtcaggtcctgctcgccgccggtgatgaagatcttgctgccgctgatgcggtaactgccgttggcctggggctcggcgcgggtgcggatgatccccaggtcggtgccggcgtgggcctcggtgaggcacatggagccggcccagcggccttcgtacatcggcggcaggtacagctgtttcagcgcttcgctggcgtgggcgtcgatggccaggcaggccccggaactcagggccgagtacagggcgaaactggaattggcggcgtagagcatttcctcgaactgcaccgcgagcatcttcggcatgcccatgccgccgtaggccgggttgccgctcaggccgacccagccgccctggatataggtgctgtaggcctccttgaaaccggccggggtgcgcacctgcccggcgtgccagctggcgccttcctcgtcgccgctgcggttgagcggggcgatcaggttgccggtgaccttggccgcttcttcgagaatggcgtcggcggtgtcgccgtccacggtatcggccagggccggcaggcgcgcccagagcttgggtgcgttgaacacttcatgcaggacaaagcgcatgtcgcgcagcggagcgttgaattcgggcattgcaggaacctcgggcaaaagtggaggttgagcggcctcggccgcatgaatcgacaacgaaaaccccgccaccttgtaggagccggcttgccggcgaagaggcccgcaagccatgcatcgccctggccgacgccttcgctggcaagcgaagcgccgcccggccagctcctacaagggctgggtaggtgtattcaggctagaggtttttcgccatgtcgcgcaggacgaacttctggatcttgccggtggaggtcttgggcaacgggctgaagaccacggtacgcggcaccttgaagcctgccaggtgttcgcgacagaaggcgatgatctcggcttcgcgcacgtcctgatgatcggccttgagggtgatgaaggcacagggcgtctcaccccatttttcatcgggccgggccaccaccgccgcttccagcaccgccgggtggcgatagagcacgccttccagttcgatggtggaaatgttctcgccgccggaaatgatgatgtccttgagccggtcgcggatctccacgtagccgtccgggtgggttacccccaggtcgccggtgtggaaccagccgccctcgaaggcttcggcggtggccgtggggttcttcaggtagcccttcatcacggtgttgccgcgcatgaagatctcgccgatggtctggccgtcccggggcgtgggttcgagggttttcgggtcggccaccatcaccccttccagggtcgggtagcgcacgccctggcgcgacttgatctgcgcccgctgttccaggggcagttcatcccaggcggcgtgccaggcgcacagggtcaccgggccatagacctcggtcaggccgtagacgtgggtcaccttgatgcccatttcctccactgcgccgatcaccttggccggcggcgcggccccggccaccatggcgttgaccgggtggtcgatggccgccttggcggtgtcgggcatgttcaccagggcattgagcacgatgggcgcgccgcacaggtgcgtgacctgatgctcgcggatcaggtcgaggatcttctgcgggtcgacccggcgcaaaaacacatgcactccggccagggcggtgacggtccaggggtagcaccagccgttgcagtggaacattggcagggtccacaggtacaccgggtggttgcccatggcccaggtcatctggttgcccagggcattgaggtaggcgccgcggtggtgatagaccacgcccttggggttgccggtggtgcccgaggtgtagttcagggcgatggcctgccactcgtcatccggccactgccaggcaaactcgggatcgccctcggcgagaaaggcctcgtaatccagatcgctgaccggctggccttcgccgtattccgggtcgtcgaggtcgatcaccaggggcgggtgatcgagcatggccacggcggcgtggatcacctcatggaactcgcggtcggcgatcagcaccttggcttcgccgtgggccagcatgaaggcgatggcctcggcgtccaggcgcacattgagggcattgagcacggcgccgatcatcggcacgccgaagtgtgcttcgagcattgccgggatgttgggcagcatcaccgccaccgtgtcgttcttgccgatgccgcgcccggccagggccgaggccaggcgccggcagcggctgtaggtctgggcccaggtgcgacggatcgaaccgtggatcaccgccgggtaatcggggtaaaccgcagcggtacgctcgatgaagctcaagggggagagggcgatatggttgacggcagtcggcgccaggccttgctcgtagatggacatggttcgggtacccggtggctgattattagctctattggccgaggcttgggtggcgtgtgctgtgtacctgggggcactgactgtaggagccggcttgccggcgaaagcgatcccacgggcctcttcgctggcaagccagctcctacgttcggcgttgcttacaggtacgcagcacacccagcgaaagctcggtgacttatccggggtcttttatatactgtttgtcgattgatatggaagtactacctgagttgtatagtggttgtactataaaaagaatctacccttccggccgcgagactgatcgaccatgcccgactctacccacctgcgtcactggctgcgcctgcagcgtgaaggccagttgccggactcgccctggctcggccacgacccgcaacccagcctgctgctggacgcccaggcgcagccgctggacctcaacccggcactgcgccagtggctggacgatgctgccactggcgatctcgcggccctgctgccggtcaaccacccggccctggtacgggcctgcctggagcagcagcgggccatcgagcaggtggaagcccagtggggggagcgcatcctgctctggagctttatccccgatcctcatcgccagcgggtactggcgcgctgccaggaagccacggaccaggtccgggccgagcgcgagtcggccaaggcccggcgcctgtaccgcctgatcaccgaaaacaccactgacctgatttcccggcacaccccggacggcagcttcctcgacgcctcaccggcctcctggaccctgctcggctactggcccgagcaactgcgcgggcgcctggcccgcagcctgttccacagccaggacctggccgggctgatgcagcgcacccgcgatgccctggagcaggacggctaccacaccatgacctatcgcatccgccatcgcgacggccactacctgtggttcgaaaccgcctgccgcggcattcgcgacacctacaccggcaccgtggtggaagtggtggcggtgtcccgggacatcaccgcccgggtccaggccgaagagaacaagcggcgcctggcggaagtggtagaggccaaccccgacccggtgctgttcatccagcccggcggcgcggtcacctacctcaacccggcggcgcggcgcaccctggggctcggcgacgggcaggcaatgccgccactggcggccattctcagcgctgcggtactcgacagcctgcaacgcgaaggctgggaccgcgctgaacacagcggccgctggaacatcgaggcgcgcctgcaaccgccggccggcggcgcctcggtgccggtctcgctgatgctcctggcccaccgcgcggccagtggcgagcgtttctattccctggtggcccacgaccagagcgagcgcgaactgcgcgaagcccagcagcgccatcaccaggacgaactggcccacaccgcgcgcctggtgaccctgggcgaactggcctcgggtatcgcccacgagatcaaccagccgctggccgcagtggtcaactacgccaatgccagccagcgctacttgcaggccctgggcacccagcccgaggccgcccgcaaggtggcccagggcctggaacggattgccgagcaggcgacccacgccgccgaagtgatcaagcgcctgcgggcctttttgcgcaagggcgggcgccgggtgcaggccttggacatcgccgaagtggcccgggaaaccgtgcgcctgtgtgcctgggaagcccaggccagccaggtgacgatcgacttgcagctggcggataatctgccgccggtgtacgccgaccgggtgctgttggagcaggtgctgctgaacctgctgcgcaacgccatcgaggccaaccgcgaagtgcatgggcagcagggctcgcacattgtgctggccgccgaggcgacggccgaaggcggggtgcagatcagcgtcagcgaccagggccccggtgtctgcgccgagcaactgccgcagctgttcaccccgttctacaccagcaaggccaatggcctggggctcgggctgtccatgagccgcagcatcgtcgagggctttggcggcgacttgcaggcccagccccaggccgtcggcctgcgcatgtgctgccggctgccggccagtgccccggccgacccctcggcaacgtcccaggcaacttcccccgcaacgacaggagcaaggcaatgacgagtgtggcgcagcaactggtgtatgtggtcgacgacgacccgggcatgctcgactccaccgtctggctcttggagtcggtggggctcaaggcgctgcccttcaccagtggccgcgagtttctcgaacactgcgacccgagcctcaatgcctgcgtgctgctggatgtgcgcatgcccggcatgggcgggctgaacgtccaggaagagctgcgccagcgcgatatccgcctcccgctgatctttgtcagcggccacgccgacgtgcccatcgtggtccgcgcgttcaaggccggggcggtggatttcattgaaaagccctacaacgaacagctgctgctggacagcgtgcagcaggccctgagccgcgccgacaaccatcagaaccagagtgccgggcaggcccgggtgcaggcgctgctggaaagcctgacgccacgggagaaagacgtgttgctgccgctggtgcagggctacaccaaccgggaaattgccgagcaactgggggtcagcatcaagaccatcgacctgtatcgctcacgggtgatgaaacgcatgcaggccgaacacctgccggaattggtgggcatggctattgccgccgggctggtagacccgctgcacctgcgctgaggctcaggccaccgcgctgcgcacgggcacgccgcccagcagccgctcgatggcgccgctcagcaggttttccaacagggcttcgcgttcacgctcggccaggggttgttcgctgaaccagctgggggcgctgttgagcaggttggccaaagcatgggccgcggcgcgctgcgcctgtgggccaaggccggtcggggtgcccagcaactgcaccagctgttgttcgtagcgctcgcgcaattgcccgacccgcagttgctggtcgggattgaggcagccgctgtcgcgctccaccaggcggaaatgccgcggcaattcccggtgcagcttcaggtgggcgcggatcagcgccggcaggcagtcgccctgcaacttgcgccgttgcaggcgcttgagggtgctgtgcagctcatcgaagaactcctcgatcaggtccagcaacaggtgctgcttgctcgggtaatggtggtacagcgagcccggggtcagccccagatggctcgccagctcacgcatgccgacctggccgaagcccttgctggcgaacagctccagggccttgtcgcggctttcggcaaagcgtgagcaacgctcagccatagtgatggaaaccccgaccggtcttgcgccccaggtagccggcggcaaccatttccttgagcagcggcgcggggcggtacttgctgtcgttgaagccgtcgtagaaggcttcaaggatcgccagcagggtgtccaggccgatcaggtcggccagggccagcgggccgatcggctggttgcagcccaggcgcatgccggcgtcgatgtcctcggcgctggccaggccttcctggaacaccaggatcgcctcgttgatcatcggcaccaggatgcggttgaccacaaaacccgggcggttgccggcggtgatcgcggtcttgcccaggcgctgggccatgtccatggccagggcgtgggtcgcgtcgctggtctgcaggccgcggatcacttcgatcaggcccatcaccggcaccgggttgaagaagtgcaggccgatgaaccgctcgggcgcgctgacgctggcggccagttgggtgatcgacagcgacgaggtgttggaggcgatcacgcactcggcgctgacctgggcggcgatctgctgcagcaccttgagcttgaggtcgaggttctcggtggcggcttcgatcaccagttgcgcatccttgagcacgccgtagtcggtgctgatgcggatccggcccagggcggcgagcttgtcgtcttcgccgagggtgcccttgctcacctggcggtcgaggttcttgccgatggtggccagggccttttccagggcgccctgggcgatgtccagcagggtcacgtcaaagcccgcctgggcgcagacctgggcgatgccgttgcccatggtgccggcgccgatcaccccgatattcgcaagattcatcttcagctcttcctttgttcattgccttcgcggagcatggcgcggttgccttgcacagcgcgttatccagcgcgagtctagagcccggcgcggtggccggcctatgccgaaagcggtcaagcgtgctggcgttttttgccataggtccagctacgggagcacagcctctacatgcgtgaatcggattcggtcgcggtctactttgtccgggtgatgacccacgccctgcgccagcagccggcgcggctggcggccgtgctgcgcgaggccggcatcgacccggcgttgctcgaccagccccgggcgcgggtgcccggcagcgcctttgccgccctgtggctgatccagatccgcgagttgcaggacgaattcttccagctcgactcccggggcatgccgccgggggcctttgccctgatctgccgcgggctgatccaggagcccaacctggaaaaggccctgcgccagtgcctgaacaacttcggcctgttcctgcgggacgtcggcgccagcctcagcctgcggggcaagcgtgcggtgatcagcctcgtcagccgctgcgccgacccgctgcgcagccactacgccgaggaaaccctgctggtgctgatcatcagcctgttgtgctggctcggcgggcgacggattcccatcgaccgcgccgattttcgtcattcacgcctgagcctcagcgatgacccgctgctgtggggcagcaacctgacctggaacgccgggcgcaccgagatcgaattcgccagccgcttcctgcgcctgccggtggtccaggacctggcctcgctcaaggtgttcctgcgcagcgccccgcaatggctggtgatccgctttcgcaaccagcacgggctgaccacccaggtgcaccagcgcctgcggggcagccactacagccagtggccgaccctggaagccttcgcccgcgaagtgcagatgagcgccagcaccctgcgccggcgcctggagcgcgaaggcggctcctatcaagagatcaaggacgaagtacgccgcggcgtcgccgtcgaactgctacgccgcaccagcgtcagcatcagcgagattgccgaactcaccggctttcaggagcccagcgccttccatcgcgcgttcaagaaatggaccggcgaaagccccgggcgctaccgcgcgcgtttccagccagcccccgcctgattccacgacatcccggctcctacggatttcgccacatcacacagccccccccgtaggagctggcttgccagcgaaggcgcccgcacccccgtaggagctggcttgccagcgaaggcgcccgcaagatcaccgcctgatccgagggcctgctcgccggcaagccggcgcctacggttggtgtggattaggccgggctgcggccgggcatctggatgccgtgctgctgcacgcggcggtacagggtggcgcgggaaatgcccagggctttggctgccgccacgggtttccagcggtggcgcaccagggcatccaacagggcctggcgctcggggctggtggcggtttccagggcgggcagggcgccttcggcctcggcgccgcgcagttgttcgggcaagtcatgcagttggatcagcggtccttcgcacaccgcgcaggcgtagcgcagcacatggcgcaattgccgcacgttgcccggccagcggtagcccagcaggcattccagcgccgcattgcccagttgcaccggcgcaccgcagcgcttggattcctcttcgacaatccgggtgatcagcgccagccggtcgctgcgctcgcgcagcggcggcagctgaaaacgcgcgccccccaggcggaaatacaggtcctcgcgaaagctgccttcggccaccagcgcttccaggtcgcggtgggtagcgcagatcacctggatatccaccgccttgcgttgcgcggcccccaggggcgccacttcaccctcggccagcacccgcagcaggcgggtctgcaaggccaggggcatgtcgccgatctcatcgaggaacagggtgccgccatcggcctgttgcagcaggccctgcatgcccttgttcgaagcgccggtaaaggccccggcgacgtagccgaacagttcgctttcgatcaggttctcgggaatcgccgcgcagttcaccgcgacaaagggccggtcccgacgctggctggcctggtgcagctggcgggcgaagacttccttgccggaaccggtctcgccctggatcagcaccggcaggtggcggtccttgacccgcaccgccaggcgcaggctctgttccacccgcgggtccagcggttcgccggtcaggctcaggcgcgggcgctgtgccgggcgccttggcgcttgcaggcggccgtgcaggttgtcggaaagggggcacagggcttcgtctcgggcgcgctgcaacagctgcgggtcgaacacctgggtgatgtgctgcggcaagcggccatagcgccgcagcaggtaatggcgcgccgccgggttgagcgcgcgcaggcagccgtcgtcgtcccaggccagcaggtaatccggctggctgtcgacataacccgggctggcatgggcccgcagcacccagtaaccctgggcgctgttcatgaagaatgcctgttcgatctcccgcgcgctctgcaccaccatctgccggatcaggtgctgggagcgacggtcatccggggagcgcaccgccgacacgtccagcacccccagcaactcgccctgggggtcgaacaccggggcggcggaacaggtcaggccgatgaacgcggcgcgaaagtggtcgcgcttgtgcacggtgatcggcgtgcgcgcggtgagcaccgccgccacgccgcaggtgccttcttcaccctccgaccagcaggtgcccaggtacagcccggccttgcggcagtcgttgcgcagggtggactccacccggtagtcgatggtctggccttgggcgtcggtgagcagcacgcagtagtcggcgtcgcgcacccggccatgcaggcgggccacttcttcgctggcgatgcgcaggaacagctcggaacgctcgcggcattccttgagcacgttttcggacaggatgcgcggcccctgcagcgagccggggtccagttggtgctgctccatggagcggcgccaggaatcgaggatcaggctcggcaccggcgcctggggcaggtgatcggcgtttttcaggacccggctgacgcagtccacatgggcttttgaatgtgcggaaagcatgaaggcctccggttgcagcttcttatcgttgtgggcgcattaagcgccgatccgcggctcccgacaagcaccgggcggcgccgtgggggcgctgagacgcaacgtctcagggtctcgccaaggccccgtgcagcctgacatgcaacgtctcaggcgcagggcttcccgacacctgcaggcgagtgcaacagccgctctgccacgggcctttgcagcggtttttccataactggcaccggccttgctctggctcttggcaaccagttctcggcaacccgcgtcgactggcccatcgaagaataagaacgaggtgcccgatgttttctcctgcctgtgctccgcccgcggtgagcccatgaaccgccgccccctcaccgtcggcatcatcgccaacccggcttcgggccgtgatgtgcgccgcctcaccgccaacgccggcctgttctccagcaccgacaaggtctcggtgatccagcgcctgctggcggccttcggcgccaccggcatcgagcaggtgctgatgcccaccgacatgaccggcatcgccgccgcggtgcagaagaacagccgcagccgccaggcccgggagcagcactggccggccctggaattcctcgacctgaccctgcgccagagcgtcgaggacacccgccaggcagcgcgctggatggccgagcgcggggtgtcgctgatcgccgtgctcggtggcgatggcacccacaaggcggtggctgccgaggttggcgacattccgctgctgaccctgtccaccggcaccaacaacgcctttccggagctgcgcgaagccaccagcgccgggctggccggtgggctctacgccaacagccgggtgcccgccgagatcgccctgcggcgcaacaagcgcctgctggtgcgcgaaccgacccgaggcctgtgcgaggtggccctggtggatgtggcggtgtcgcccctggcctttgtcggtgcccgggccatcagccgcgccagtgatctggccgaggtattcgtgaccttcgccgaaccgcagtccatcggcatttccgcgctctgcgggctgtggctgccggtgactcgccaggaaccccacggcgcctggatgcgcctggacccgcaatcccccgaggcgctgctggtgcccctggcccccggcctgctgcagggctgcggggtgctggacgccggctacctgcaacccgatgtgccccagcccctgtgcctgaacgccggcaccctggccctggatggcgagcgcgagatcgaattcaacccccacgaccggcccacggtcaccctggatgccggcggcccgctgagcatcgacgtcaacgcggtcctggaacacgcggcgcgccagcgcctgctggccatcgaccgcgatcaccggcaacaccccgtgaaccttcagtccgaagacaccctggagaataaaaatgtcgacacccctgaccactgaccagttgctccacgcctaccgggtgatgcgcaccatccgcgcattcgaagaacgcctgcacgtggaattcgctaccggcgagatccccggtttcgtccacctctatgccggggaagaagcctcggccgccggggtcatggcccacctgcgcgacgacgactgcatcgcctccaaccaccgtggccacggccattgcatcgccaagggcgtggacgtctacgggatgatggcggagatctacggcaagaagaccggggtctgccagggcaagggcggctccatgcacatcgccgatttcgagaagggcatgctcggcgccaacggcatcgtcggtgccggcgcgccgctggtggtgggcgcggccctggccgcccggctgcaaggcaccgacggcgtgtcggtggtgttcttcggcgacggcggctccaacgaaggcgcggtgttcgaggccatgaacatggcctcggtgtggaacctgccgtgcctgttcatcgccgagaacaacggctacgccgaagccacggcctccaactggtcggtggcgtgcgaccacatcgccgaccgcgccgccggtttcggcatgcccggagtgacggtggacggttttgatttcttcgccgtgcacgaagctgccggcgccgccgtggagcgggcccgggccggggaggggccgtcgctgatcgaggtcaagctgacccgctactacggccacttcgagggcgacgcccagacctaccgcgcccccgacgaggtcaagcattaccgcgagcacaacgactgcctgatgcagttccgcgagcgcaccacccgttcgggcctggtgcaggccagccagctggaccagatcgatgccgacgtggaagcgctgatcgaagacgcggtgcgcaaggccaagtccgaccccaagccgagccccgccgacctgctcagcgacgtgtacgtcgcttatccataacgccccacagaacaacaagagaccaccatcatggcgagaaaaatcagttaccagcaggccatcaacgaagccctggcccaggaaatgcggcgcgaccccagcgtgttcatcatgggtgaagacgtggcgggcggcgccggcgcccccggcgacaacgacgcctggggcggcgtgctcggggtgaccaagggcctctaccatcaattccccgggcgggtgctggacaccccgctgtcggagatcggctacgtcggcgccgcggtcggcgccgccacccgtggcgtgcgcccggtgtgcgagctgatgttcgtcgacttcgccggctgctgcctggaccagatcctcaatcaggcggcgaagtttcgctacatgttcggcggcaaggcccagaccccgctggtgatccgcaccatggtcggcgccggcctgcgcgccgcggcccagcactcgcaaatgctgacctcgctgtggacccacatccccgggctgaaagtggtctgcccctcgtcgccctacgacgccaagggcctgctgatccaggccatccgcgataacgacccggtgatcttctgcgagcacaagctgctctacagcctgcagggcgaagtgccggaggagtcctacgcgattcccttcggcgaggccaacttcctgcgcgacggcaaggacgtgaccctggtgtcctacgggcgcacggtgaataccgccctggacgcggcccgcagcttggccgggcgcggtatcgactgcgaggtgatcgacctgcgcaccaccagcccgctggatgaagacagcatcctggaaagcgtggagaagaccgggcgcctggtggtgatcgacgaagccaacccgcgctgctccatggccaccgacatctcggccctggtggcgcaaaaagccttcgcttccctcaaggcgccgatcgaaatggtcaccgcgccccacaccccagtgccgttctccgacgccctggaagacctgtacatccccgacgcggcgaagatcgagaaagccgtgctcactgtgatcgagtggagccgctgatgagccagatccataccctgaccatgcccaagtggggcctgtcgatgaccgagggccgggtcgatgcctggctcaaggaagaaggccagagcatcagcaagggcgatgaagtgctggacgtggagaccgacaagatctccagcagcgtcgaggcgccgttctccggcatcctgcgtcggcagatcgcccgccaggacgagaccctggcggtgggcgcgctgctgggcatcgtggtcgatggcgaggccagtgacgccgagatcgacgcggtgatcgagcagttccaggccagcttcgtgcccggcgacagcgccgatgaagatagcggcccggcgccgcagaaggtcgaactgggcggccgcctgatccgctatttcgagcgcggtgaaggcggtacgccgctgctgctggtgcacggctttggcggcgacctcaacaactggctgttcaaccacgaagccctggccgccgggcgccgggtgatcgccctcgacctgccgggccatggtgagtcggccaaggccctgcaacggggcgacctggacgagttgagccaggtgctgctggcgctgctggatcacctggagattcccgtggcccatctggtgggccactccatgggcggcgcggtgtccctcaacaccgcgcggctggcgccggaccgcgtgcgcaccctgaccctgatcggcagcgccgggctgggccgcgagatcaacggcgactacctgcaaggttttgtcgaggccagcaaccgcaatgcgctcaagccgcagctggtgcagctgttctccaacgccgagctggtgaaccggcagatgctcgacgacatgctcaagtacaagcgcctggaaggggtgcaggcggccctcgggcagttggccggcaacctgtttgccgacggccgccagcacgccgacctgcgcccggtggtgcaggacggcccgcagccggtgctggtgatctggggcagcgacgaccggatcattccggtgagccacagcgccgatctcaaggcgcagatcgaggtgctgccggggcaggggcacatgctgcagatggaagcggcggagcaggtcaaccgcctgatcctcgactttatccagcagcactgaggtgttctgcggcggccagggggccgtcgcagctctacaagaataaggagaagccaagatgagtgtttccctcaagcctgtttccagcatgcgtgccgcggtctggcacggccgccacgatatccgcgtcgaagacgtaccgctgcccgacgcgccgcctgccggctgggtgcagatccgcgttgactggtgcggcatctgcggctccgacctgcacgaatacgtggccgggccggtgttcattccggtggacgcaccgcacccgctgaccgggatcaagggccagtgcatcctcggccacgagttctgcggcgaaatcgtcgccctgggcgaaggggtccagggtttcagcgttggccagccggtggccgccgacgcctgccagcactgcggcacctgctactactgcacccacggcctgtacaacatctgcgaaaacctggcctttaccgggctgatgaacaacggcgccttcgccgaattggtcaacgtgccggccaacctgctgtacgccttgccggccaactttcccgccgaggccggagcgctgatcgagccgctggcggtgggcatgcacgcggtgaaaaaagccggcagtttgttggggcagaacgtggtggtggtcggcgccggcaccatcggcctgtgcaccatcatgtgcgccaaggccgccggcgcggcccaggtgatcgccctggaaatgtccggcgcgcgcaaggccaaggccctggaggtgggcgcgacccacgtgctcgaccccaaggagtgcgatgccctggccgaggtcaagcgcctgaccggcgggctgggggccgatgtcagctttgagtgcataggcaacaagcacaccgccaagctggccatcgacctgatccgcaaggccggcaagtgcgtgctggtggggatattcgaagagcccagcgagttcaacttcttcgagctggtggccaccgagaaaacggtgctgggggccctggcctacaacggtgagttcgccgatgtgatcgcctttatcgccgatggccggctggatatttcgcccctggtgaccgggcgcatccagctggaagagattgtcggccagggcttcgaggaactggtcaacaacaaggagcacaacgtcaagatcatcgtgtcccccggtcagttgcgacgctcgtaaacgagcgatttggcgatggccggccgcggccatcgcacttttttacagttttgcgttctatcacctggcctggccgcgccgatccccgccgcagcccgtgaattccgacccggtgccgatggcctacgctccctcttcttgcatgatgcaaacccgctttcagcgcctgttcctggtggcgctgctgatgcttggcgccacggcctgtacccaacagcaagggcgcgattttgccaaacagtttcgtgaaggcaagcctgacgaattcttccagaccagcgtcgatcgcatggccaccatcggcatgcgcgacaacctgcagagcctgtacctgctgatgggcaagctctacctgcgcaaccccagccagtggcgcaagtcgggtttccccgatggcaccacggcccagcgcgagatccgcaatgccatcgaaaagcgccagccgctgccggccctgggggatcgtcgcgacctggcggccctgagctattccctgagcccggagttccagggcgatcgggtgggggcgttcatctatgccattggcagcatgctggtgaccgcccatggcgggcgcaccgagttctacatgaccgataccatcgacccgcagttcatcagcaacgccgcgcgcaatatcgagaaggccacctggctgctgagccagcgccaggatgccaatggggtgctgttgctgttctccaacgagatttccgaggagggcagcaacctgagttttgccgtggagttcggcaagatcgtcgcgcgcctggatttgctcacgcagattctcgatgagcgctaccggcgggtggggttcaattacgcgcagagcctgctgctgatgaatttcttgccggtgcagtaggagctggcttgccagcgaaggcgcccgtccaggcgctgcaaggcccaaggacctgttcgccggcagccggctcctacacggcgtttggcttggccctctctcaggtcattccgagttttttacgcgccatcaacagggctacgacgctcaacgctgccgccagcatcaggtagaaactcggtgccagctgtgagccggtcagggcaatcaatccggtagcgatcaccggggcaaaaccgccgaagatcgtgacccccaggttgtagctcagggacatgccggtgacccgggtgcgcaccgggaagatgtccgccatcagcgacggcagcggggcgaagtacaccgccttgatcagggccatcagcgccaccaccagcagcagcgaactggcactggggcggctgttgagccagaagaacgccggcaggatcgccacggcatagaccaccgccgctgccagcatgattttcaggcgccctatatggccggacaggtagccgacgatgggcgtgatgatggtcaggatcaggccgccggtaagggtcgccaaaaagcccgaggactgggcgatgttcaggctggtggcggcgtaggagggcatgtactggatcatgtagttcgaggcggtcgacaccaccagggtgcccatggcgatcagcagcagggtcttctgggtcaccagcacttcgcgcaaagggcgttcccccggttcgctggccttgaagtccggcgactcgtccaccgtacggcggatgtacaggcccaccgggcccagcagcaggccgaagaagaacggcacgcgccagccccagtcgagcatctgctgtgcatcgaggtgcttggtcaggaagtagccgaaggtcgaggcgatgaccacgccaaagccctggctggcgaattgcaggctggcgacaaagtgccgggtgcgcggcggcgcgtgttccagcatgaaggcggtggaggtgccgaactcgccaccggcagagaagccttgcagcagccgggccaggaggatgccgatcggcgcggcgaggcccagggtggcgtagctgggcatgatggcgatcagcagggtgcccgtcagcatgatcaggatcgacagggtcagggccttcttgcggcccttgcggtcggcgtaggagcccagtaccgccgcgcccagggggcggatcaggaacgagatgccgaaggtggcgaaggccagcagcagggagaaggtcgggtccgcggtgggaaagaacacctggctgatggtcagggcgaagtaggcgtagatcgacaggtcgaaccactccagggtgttgccgatcagggcggcgcctatgggtttccacagggccttgggttcgacccggggcgcggtcgaggtggtggctaacgctgtcatacaggacgctcctgcttattgttggattttgagcgacaggtgcccggccagggccgggcggggtaagcgatggcctcaggtccggggcaggtcccccaggtcccagaacagggccgtcatgatctccaggccttcacgaatcaccggcagcagcaggtgttcgtccggggcgtgctgcgagcagccggggtaggagtggggaatccacagggtcggcagccccagcacttcggcgaagatgtcgttgggcagggtgcccgccaggttcggcaacagcgccaggcgctgcgcactgctacctgcaatcgaggtcttggcgaacctgacccaggggttgtccgggtccaggcgggtggccggggagcagcgctccatgacgatctccacctggctgaaaccttcacggtccaggtgcgcccgcagcaggctttccagctgctgccagggcgtgccgaggacaaagcgcaattggcagaaggccacggcccggggcggaatcgcgttcaccggcttttgcgggttgccggcgctgaaggcgaggatttccagggtgttccaggcgaacaggcgctcgcccagggtcagccccggttcgccccagtgttcgtccagagggcggcccagggactcgcgctggatctccaggtcgacgatggcggcgcgcacgttgtcgggaatgttcgccggcagcagttcgcggcagcggatgcggccgttctggtccaccaggctggccagggcgttggccagtaccacgccggggttgctcatcaccccgccccagttgcccgagtgcaggcccttgtcccgggtgttgacctccaggcggaactgcgccgagccgcgggagccgaggaacagcgtcggccgggcctgattcagccgtgggccgtcggaggcgatgaacacatcggcccgcaacagttcgcgctgctcctggcacagctggcgcagccccggggaaccggcttcctcacccatctcgaacagcagcttgaggttgaagcccaggcgcccgtcgcgggcctttagcacttgctccagggcggtcaggttgatgctgtgctggcccttgttgtcggcggtgccacggccgtaccagcgttcaccctccagggtcagctgccagggattcaggcccgtgcgccactgttcggcgtaacccggcaccacgtcgccgtggccgtagctgagcactgtgtgcagggctgggtcttccaggcgcgtggcaatcaggaacggcccaccggcctccgggttggccagcagctgacagtcgaagcccaggcgctgcagtagcggctggatttcctccagcaggtagcgcggcaactcgctggcgtgggccgggtcctggctgtcactgcgaaacgccacccggcgtcgcaggtcctgttcaaagtggccgtcgtccaggtactggcgaacgccggcgagggcagcttgcttggtcattgcaactccggtcgggcaaggggaacggacccatggtggagttggcaaaggcatctgtatattgctgttttttgaaggcatgattctaaaaattggatgcctggcaaggggacgcacatggactatcggtattttctggcggtgatcgatgcgggctccctggccgcggcgggggagcgtttgcacatcgcccccagcgcggtgagccggcagatcgccctgctggaagagagcgttggcgtggcgctgttcgagcggcgcccgcggggcatgcaactcaccgccgctggcgaagccctggccgaacacgcccggcgcacctggctggaacaggagaacatcctcgccgaactgcggggcgagggttacccgggggccaaggtgatccgcttggtatccaccgagggcctgtcgcggtatttcctgccccgggtgctgctggagcattaccgcctgcggcccggagccgagtaccgcctggacgtgatctcgccccacgagtgcgtgcagcgcattcgccggggcgaggcggacgtggggctgaccttcagcaccgagcccgtggagggggtcaacgtgtgctactcgatccgctcgccgattcgcgccctgatgcgcgccgggcatccgctggccagcgccggggtgctgtcactggagcaggtgcaggcgtttcccgtggccttgacgcccaagggcaccacccagcggcaactgctggacctggtgtgccagatggacggcctgagcttcgatcaggtgatgacctgcaacttctccggggccctgcatgagtttgtgcgccagtccgacacggtggcctttgccagtgccatcagcggcgaagggcaggggatggatgggctggtatcggtgcccctgtccaacccgcgcttgggcgatcgggacattcaggtgctgaccatgcccaagcgggtgttgccggaaagcctggcgttctttatcgacaccttgattgcgcggttgcagaaagagggctgagggaggctgctgaccgttctcgccgagccggacacgctcagggcgctgggcgagttcctgatcaaggcttcagccgacatggcggccaatggcctggagcatgtgcacctgcaagacgtgatcgagcacttctcgcaccagcagcatgtggatgtgattgccctcgaccgggcgctgatcaagcctgcctgaggcagcgccgtgcccggcccggcttacatgttgcgaatcctctcgtcgaccctgagctcgaactcgaagccaatgcccacgtcattcagggtttcggtgacccgggccatgaagtactcggcctgcgcgggcaattcatacaagccaatgacccggatgatcgggtacttgccataggcgccggggatctcggtgtagatctcgccactttcgatgaaggcgatgtagctgttgattttctcttgcagcacgagcaagtgctcgccttgttccgccttgtcgccccattccaggtggtcggtcatcaccagtagcaggtcgttgggagcctccttggagatcccccagaaatcgatgactttgggattggtgacggacatggctgatccttcaggcggcgggggcggggattgtattacgtgccccggggttcgtggcggtgctgttgcgcgtgcggcatggatgggcatgcagcgcacgatgcaggaactgttctagtgaccgggccgggccctgaaggcgagaaccgccccggcgattttccaacaaccgttgcgtaatggatagttggccataggatgtgcgcgttcccgatgatgatcgggcccccatcgtcgtgatcctttactcagtttcaaggttgctgcatgaatccggatgacttcaagaaattcaatatcggctccattgttcgggccactcaaagctctgctgcgctcaaacaccagctcggattcctgatcagggttctggaggaactggatatccagttctccgactgcgagcaggatcctctgcgcctggcgaaagcctatgttgccggggaaattcctgctgaacgctgcaaggcgcaggctgcactctggtgggcaaaaatcgatgagcaaggggcaatacgagggttcaaggatcgggaggtattgaaggcacgactggccatttgtctgctgtcgaatgatgaggacgatatttccgtgttgggagataacctctcgtggtttcttgaggtgttgggtttcttgaaagtggaaccgatgaagccaattgaaatgatgagagagtatttcgagttcaaatagaagttcattcgcgcccggctcgctctcggtgccctggccggcagcctgacgacaggcaagaccccatgcgttttgctggctccccccgtcacgctggtggggtgctcgacgcgttttccatgagccgcatgtggatttcattgcgcttgatcgggcgctgatcctgcctgcacaaggtgttcctgcgcgcggctcaatcggcgagcgcgcagggggcgagctacaggctgggagcctcttcgttcactttcagctcgaaatcgaagccgatgctccgttcatggaggatctcggtaatgcgtttgaaaaaccaatcgacttgctcgggcagttcgtgcaaacccagtacccggataatcaggtgcttgccgcaggcatcgggaatttcgctgtagatctcttcactttcgatgaaggtgatgtaggtgttgatcttctcttgcagcaggagcaggtgttcgctgtgtgccgcttcgtcaccccattcgagggggtcgaccatgatcaacagcaggctgtcgggcgcggatttggggatggcccagatatcgatgacttttggattggtgatggacat

SEQ ID NO: 2 - DNA, 52750 bp, Pseudomonas protegens
  misc_feature 1..52750: note = Pyoluteorin biosynthesis gene cluster
  source 1..52750: mol_type = genomic DNA
agtgttcaacggcgttcgggtcacccgccttttgcgcgagaacgggcgggtctgcggggtgcagatcgaggacagcgaaggcgggcagccgcagcaggtgcgctgcgcggtcctggcggtcgctaccggcgcctgggccgagcgcctgcgcccggcggaccgcgtgcgccaactgcggcccttgcgcggcagccatctgctgctgccgggctggcgcttgccggtggcccaggccttcagcttcatgcatgccgaggatggccggccggtgtttgtcttcccctgggagggcgccaccgtggtcggcaccaccgacctggatcaccgcgatgacctggaccagagcgccagcatcagccaggaagagcttgagtacctgctggccgcctgcgcccagcagtttccccaggccctgatcactgccggcgacgtgctgtcgacctgggccggggtgcgcccggtggtggccagcagcgacggccagcagggcaagccttccaatgaaacccgcgagcatgtgctgtggcaggaacccggctgcgtgaccctggccggcggcaagctcaccaccttccggccccaggccatcgaagtgctgcaggcctgcggggcgatgctcgggcgtgaagtggtggacgacggcgccccggtcttcacgcctgtggcgcccctggagatccccggtctgagcggcgcccaggtgcggcgcctggctggccgccacggccgccatctgccgcggctggcgcagtgggtggcgaccctgggcggtgattgcgtcggcgccagcgacaccctgtgggccgaactggcgtttgccgccgaggcggaaatggtcctgcacctggacgacctgctgctacgccgcacccgcatcggcttgctgctggccgagggcgccgcccagtacctgccggccatccgccgcctgtgccagccgcagctgggctgggacgacgagcgctggcaggccgaggaacagcgctaccgggccctgtggcaacgccatcacagcctgccgccactcgcctaacgatgctcaaccaagagagccccatggataaccacaacaacaataagcaggactacctgctggccatcgacaacggcacccagagcgttcgtgcgctgctgttcgacctcaaaggcaacctgctgggcaagggcaaggtggagcttgaagcctatttttccagccggcccggctgggccgaacagcacccggactactactgggccaagctcggcgaagcctgccaactgctctggcagcagaccggcatcgaccgttcgcagatccgcggcgtgtccctgaccacccagcgcggcaccctgatcaacgtcgatgcccagggccgcgcgctgcgcccggcgatcctctggctcgaccagcgccaggccgaggtcgaaggcaagatcaaggggccctggggctggctgttcaagttgatcggtgccgaggccacggtggattattttcgcgcccaggccgaggccaactgggtggcacaggagcagcccgaggtgtgggccgccaccgacaagttcctgttgctctcgggctttctcacccatcgcctgaccggcaatttcgtcgactcggtgggctgctgcgtggcctacctgccgttcgactacaaacgcctgcgctgggccgcccccagtgactggaagtggcaggccctggcggtgcgccaggagcagttgccgaccctgctcaagcccggggaaaccctgggccggatcaccgccgaggccagccgccataccggcatacccgaaggcctgccgctgattgccgcgggtgccgacaaggcctgcgaagtgctgggctcgggcgtccaggactccaccaccgcctgcctgtcctacggcaccacggccaccatcaccaccacccgttcgcgctacctggaagtggtgcctttgataccgccgtacccggcggcggtgccggaccactacaactgcgaggtgatgatctttcgcggctactggatggtcagctggttcaagcagcagttcggcctgcgcgaagtgcagcaggcggcggagcaggggctggagccggagcagctgttcgacgccctggtccaggcggtgccgcccgggtccatgggcctgaccctgcaaccctactggtcgccgggcatccgcgagccgggcatggaagccaagggggcgatgatcggctttggcgacgtgcacacccgggcgcacatctaccgggccattctcgaaggcctggcttatgccctgcgccagggcatggagcgcatcgaaaagcgctccaggctgaagatcagccgcctgcgggtggccggcggcggctcccagagcgacgcggccatgcaactgacggcggacatcttcggcctgccggtggagcggccccatgtctatgaagcctcggggctgggtgcggtgatcgcctgcgcggtgggcctggggctgtacccggacttcgccacggccatcagcgccatgacccgggttggcgcagtgttcgagccccagccccaggcgcagcagatgtaccagcggctgtacagcgaggtctacctgcgcatgtaccgccagctcaagccgctgtaccagagcattcgcgagattaccggttacccggcttagcgaaagcgccggccagcggccggcagggggggcgaacctctgcggggtaaaaagttcttccaggcgccagggctgatctatcgtgatcaaggggcagggccccttcgaccccccaacgcctgcgagacgagcatgaaccgccaggaagcgctgaacatcctgtggaagcgactgttcctcatctttgtgctgctattggcggcatccctggtgtcggtggccatggccgatggctacaccattccctcggtggtgttcattgccggcaacatcggcggctatgtcggttttcaccgccagctttccagcctcagcgaggaggaaatcattgggctgtgcagttcctggttcagcctggtactgccgtcattcatcggcggcatcctcgcggggctgctgtatatcctgttcatttccggggtggtgcaaggggagctgtttccaaagatcgtcgccgatgaaacctgtcgctacagcgagaacagcttctatgtgatcttctgccagcacgccggtggctatgccgcctacgccaagctattgttctggtcctttgtcgcggggttcaaccagaactacgtggtggacctgatcgacaacatcaagggcagccagaagacggagaatcagccatgagtgaacagagcaaagggccggcgtcgtacttcccctccattgaaaagcactacggccagcccatcgggcactggctggagctgctggcaacggtcgacggcaaaaagcatatggagctggtggcctggctcaagaccgaacacggcatgggccatggccacgccaatgccctggtggcgcacttcctggcggccagcaagggccgctagtccccggggcgccattcttcgccagtgaaagcctgactgccgacatccgctttcctggcgaaaaaagccaaattcccctgtgttctcttcgttctttcctacgtcgcaccttgcattgccctctttctgcctgtcggagttttctcgatgtcttcgccaaaatccttgcccacggccctgttgggcctggccctggcctgcccggcatttgccgaaacccgtgccgtggagctggctccggcgcaggtcctggggcaggaacagggcggtgaggggcagccggcgcaggaggccgcggcgcggctggcccaggtgcccggcggcaccaatgtggtggacatgcgccgcccgctgcagggccgggtggccagcaaccaggatgtgctggcctaccagcccggggtctatgcccagtcggcgggcaacgaaggggtgaagatctcgatccgcggctcgggcatcaaccgcgcgccgggtgcccatgcctcggggctgtatacgatgctcgacggcttgcccctgaccggccccggtggcacgccctatgaattgctggagcccttgtgggtcgaccacgtggaagtgctgcgcggcgccaacgggttcgaccgtggcgccctggccctgggtggcgccatcgattacgtcagccacaccggctatgacgcaccgctgttgcaggtacgctacgccaccggtagccacggttatcagcagcgtcaggtcagttccgggcaagtgctgggggatttcgactattacgtgtcactgaccgactccaatgccgacggctaccaggatcacacccgcagccagagcaaggggctgatcggcaacttcggctaccgcttcaacccgaacctggaaacccgcttctacctgcgctaccgcgagaccgacaacgatctggccgggcgcgtgaccaagcgctccatcgaacacgacccgcgggcggccaacccggcctacgtcagccgcgacgaccggcgcaagcagccgggcagtacctttgtcggcaacaagaccacctattacctggacgacgattcgagtatccaggccgggctggtgtaccacgactaccccatggacctgcgcgaaggccccaaccgcctgaaggtggcctacagcgacgtcagcggcaccctggactacaagcgccgcgacagcctctggggcctggaaagccgcagcaccctgggcctgcgggtgaccaagcacctgcccaatgacggtgccagcgagttcgtgcggattcccaccggcaataccgcgaactacgcgccgggcacgcggatccgcaacttcacttaccagggctcggacaccgtcctgcacgttggcaatgacctggagatcgccgacgacctgtggctgaccaccgggctggcggccatctacacccgccgcgaaagcgcggtgacctaccccgagggcggcggcaagaccagccttggcgactgggactacgcgccgcgcctgggcctgcgctaccagctgaacccggatctgcaactgttcgccaacctcagccgctcggtggaagcgccacacccctggtcgctgatctacagcgccaatcagcgttttccggccggcagcggcgtcgccaccggcgcccagcgcgacccggtcaagctgcgcaatcagaccgctaccaccctggaactgggcgggcgtggcgatagcagcctgggccagtggagcctggcctggtactacgcccaggtgcgccacgaactgctttcggtgctgccggatgccaacgccatcacgccttacgaactcaacgccagccccaccgtgcaccagggcgtggaagccagcctgcagagcgagctgtggtcgcgcccgggcgtcggccgcctgagcctgcgccaggcctataccttcagtgatttccactaccgcgacgatcagcgtttcgccgacaaccgcctgccgggcctgcccatgcactactaccagggcgagctgcgctacgactggcccctgggcttcttcgccgcggtcaatacccagctggtgtccaaggtggcggtggattacgccaacagctactacgccgatccctatgcgctgttcggcgccaccctgggctacaacgcgccgaagaacgactggcagacctgggtcgatgtgcgcaacctgaccgacaagcgctacgccgccaccgtgaccccgggctatgacgacaaggggctggacgcggcgcgttcaacgccgggggaagggctgggggtgtacctgggggtgtcctggagcctgcgctgaaagcagggccgcgagcgagccgacaccccccaatgaaagtgcccgcagcagcgggcacctggttcatgccgggcggtgtcagcccgggcggcagtgatttaccactggcccgagaacacgcaggtcagcgcttcgatggcgctgaacttccacagcgagtcgccgcagtcgctggcaaaggcgtgggcgttggaggcggtgagggccagggcgatggccgaggcatagacgattttcttgagcgacatgggaaaactccttgttatgaatccgcacgttcttgtaggcgccgtcctgaagggggggggctgctgcggactataggcccgtgctgcccccggcactattagcccgagtgtgctaatggcccatgcgcatcactgtgccgcccggcccaggctcagttgcgaaaatacagctcgtccagctgccgccggcgtatcaccaggtactcctcccagaccttggggaaatcctcgtcccgggtcagggggaaggtcaggtagtaagtccgaccgctgacactgatgtactcccgcacccgcttgtacgccggctgttccagcagcgagatgtattcgtcataggcgtagtggtagtgggaaatcgtcacccgatggggcgcgatcatgcagcgggagaagtcgatcacccagccatggctcatgtcgctgcccaggtagccgtagaccccatcctcggagaggctgatggcttttcgtgcctgtaccccatcctcatcgttgaccagcacgatctggctttcgaagtagcggatggcctgttgctcggggccgcgcaactgcaggttccagccgacggcgcccatgcggaactcgccctgctcggtatattcggcagtccagccgttggcaaggctaatgcggtgtgtggtcattgtcgtcctggtgatggctgtccctgaaggctggggattgtacgtcagccgctgtgcgaagcggcagggcccgtggcgcccggtcagtcatgccagcatgcgagctgtcctgtgactgtggccgtgccctgtggctagttcgccagtgcgtggcgcagatgcgccagggcccgggtttcgacctggccctggtcgctccccggactgaagaccgcgcagcaggcgctggtgccggcgacgttggcgcgatagaccgccagcgtgctgccggggccgctgccggtgtggccgtagagcgtcaggccagactcgacagtgcccaccatcaggcccatggcatagccgggagcgacccagggccggccggccatcggcccgcccagaacctgggcctgctgcatgccagccagcagtgccgggggcaatagcgcgccgctcaacagccgatccagcagcagcgccgcctccgccaggggccccaccagcaggccgtggtagacccagccggggtggtagtcgcccaactggcccaggtccacatcccgcaggtcttcgcgggtcacagcgaggcgcgtgcgagacaagcccaggggcgccagtacccgttgttgcagggcaaggtgcaggggcaggtccgtcgcctgctggatcagctgcgccacgaacaggtagcccacattggagtagcgccactggctgccgggggcgtagcgcaggcggctggcgtccaggcgctgcagcatctgtgcaccgggccatgcatcctcgttgcgcaaaaccgcgtcgtggtagtcgctcagctcgctgtagtccgccagcccggcctgatgccgcagcaactggcgcagggtgaaggggccctggggcagcggctcatccagccccagcaggccgtcgcgcaccagggtcagggccgctgcgctaagcacggtcttggtgaagctccaccagggcaccagtggctggggttgctggggggccagtggttggccgttgtggatcagggcatgaagcacggaggggatgtccttgatgggctgtgttgccgcgggctgttgggtcaatcaggccgtgagcggccctacagggtacggcataatgcgcggccgttttggctaaacctaggaattgatcaggaggatcaggctggctatgtacagacttgggcacatgatagacaaccagtgggtcgagcacagttatccgccggtattcgtggtggcccccgttggcgaggggcagcgcgtggtggcgggcgttccaggctcggacccgcaggtcctgctcagcctgctgggctgcctggccgaaccgctggtgctgctgttcgtgctgcacacgccccgggacgaatcgcaagccggacgctactggagcccgcagctgtcgcgtgaggacgttcaggatttcatctacgacttcaagccactgctctgtggcgacagccgtttcgacctgtgggtccactcgcccgagcaacaggccaccgtggtctgggaccggcacaacctgatctatggctacgggccgctggaagactacgccacggccctgcgcgcactgggcttcggccatggcgagccgcagttgcccgtgccccatacccaccactaccacccgcagttggacgacctgtgccggcagttgctcacccacttcgattggcagtactcgccattgcgacctgaggatgagcagtgatggtgcaatcaagcccannnnnctcatgctcggtcaatcttttgaattnnnnnnnnnnnnnnnnnnnnnnnnnnnnnnnnnnnnnnnnnnnnnnnnnnnnnnnnnnnnnnnnnnnnnnnnnnnnnnnnnnnnnnnngcncatgcgggactcgnaggacnggcggcgtttaggaacggggccgttgcggcccccgatccggtttgtcactggccgctgtgcaaggccggcgtgcgcggtggaatcaggcgcttgctgccggtggcctcgaaggccgaagccaggcgcagcaggctcgagtcgtcataggcgcgcccggcgaaggtcagccccaccggcatgccgatgtccgccatcacccccatgggcacggtaacggtgggcacccccaggtggcggatggcgaggttgccgttggccacccacacgccattgctccaggcgatgtctgccgactcggggttgatatcggcatcggccgggccaacgtcggccacggtggggaacagcacggcgtccagccccaggcggtccatccagtcttccaggtcgatgcgccgggtctgctccaggccgcgcaggccgtcggccagggtgttgatctggtcccaggtcttgagcccgcgcttggccatgttcacgtattcgtccatgccggcggccaggtcgtcttcgcggttgggcagggtgccggggtcgtgggggaagatctgcggcccgtcgacatccgccaggcggttgagcttggggtcgtcgttggcccgcaggaagtcgtcgaaggcccagcccgagagctcccacagctcgtcgtggaggaactccttgctgaccaggccgcggttgtacacggtcggcgcgccggggcggtcgccctcgcagttggacaccagggggaagtccacttccagcacttgcgcgccggcggcttccagggcctggcgcgcctgctcccaaaggctgatcaccgagggccgggtatggatgcgctggccggtggggccgccgatgcccggtttctcgctggtgccggcctcagggtcggcattgatgtacatgcgcggcacgccaaggcgcttgcccttgagcgccgcggcgccggccgccagttgcgggtaggaggccgggcgcaccgaagcgacgctggggatcggcacccagggttgcaggcgccacaggtcgccgcgggtgtccgggtcttcggccaccaccacgtccagcacttccaggaggtcggccatggtccgggcaaaaggcaccaccacgtccatggtcggggtcagcggccagttgccgcgcaccgagatcaccccgcgcgacggggtgtaggcgcacagcccgttgttggaggccgggccacggccgctggaccaggtttcctcggccaggccgaaggcggcgaagctggcggcggtggcggtgccggcgccattggacgagcccgaggcgaagggcgcggtcaggtaagcggcgttataggggctttcggcgcggccatacacgccacgctgcatgccgccgttggccatgggcggcatgttggtcttgcccaggcagatggccccggcagcccgcaggcgctcgatggtgaaggcgtcgcggtaggccaccagctcggcgaaggccgggctgccggaagcagccgtcaggcccttgaccaggtagctgtccttggcggtgtagggaatgccgtccagcgggcccagggtctggcccttggcgcggcgctcatcggcggcccgcgcctcgttcagcgcctcggggttgcgcaccaccacggcgttgagggcggtggcggtgttcgggccgtcataggcctcgatccgcgccagataggcctggaccagttccaccgaggtggtctgcccggattccagggcagcgcgcaggtgggcaatggagacttcggtgacttcgatcatgctgctaccgccgttggctggtgggtgatgaggtgcagagggcgcaacggggcgcgggttttcgggtattggctgtgcatggtgattctcgtggtacggctggcacgacaagggtggcatgggaacgctgggggcctttttagcatctagtgcggggcagaggaatcggcttgatggattattgcttcaggccagctgcgccagggcctgcgggctccacgttgaactcgccaaggtagtccagggttgccgtgcttgcgggctggcgttgattggcgccgccggagatcgatttgtggtcaccgtaaatcgactcgcagccaacgatgatgaaatggctgtagccggagtcgacgcgcgtgcgcagctgctttttttcggcgtcgctgcggatcagcgattccaggtattgcgggccgtcttcactgccgatcagctcgacgaaatcggagtccatgtagtaccccagctcctgtttgaggccccagctggggttgcgtgcttcccaagcgctgaactcggcatcgctggcgtcggccgcgggttcgggctcccactgttcgaaggcaaaccgttcggcctctgcctggtccgggaaccgggcagcgaacaggtagaaaggaaagtaggcatcggcttctgacatggacggctaggctccttggttcagatcaggctggcgcgctgcaaatggctgacatgagcatggccggcggcgctcaagcgcacggccaggtcttcccgggcctgggccccgccgctgcgcacgtactccagctccgcggcggtgatcagcaccaccggcaccagggtcacgggggacagcggcatgtcttccaggcgtgtcgggaagtccggggccgggccgcccaccagtaccccggcgcagtcgtcggcgctgatgaaatgcgctggcagttgctcactcatggcgcgggactggctgacgccgggcagttccagggacagcaccccgtagcgctgcaggcgtgcagtgaggccaccggcatcggccgtggtgttggctacgcagcgcagcagttcgaaagcccagctatcggcgaacggatagacatcacccaccgcgccctgggcatggggtgggatgtccggggtttcgacgaacagttccatctcgaaaccattgcccagatcatcacctgtgaccccatcgaacggatcggacaggccctcggtggcgagaatgatgccgttgttgcgacgcacgatgcgataggcctggcgcgtggagggccagtgcgagccgcccatgaaactggggctgatggcgaagcccaggacgtcttgctcacagtggccgaccgcttgccagtggcgatcgaggcaggcggcaccggcctccatcagggcaatgccggccagctctgcttcggttggctggtactgggcgtagggctcctctgcggcggccggctcgccggcgggcagggtgggctcggccttttgtggcgccggctttttgaagacgctgagcagtttttccaggaaattcatcgggcgatccttgcggggcaagtgcgttgcacaaggcgcacagcacaggggtagagagagggggcacattgtgacgggtttgccatgcccatggaaaccgacggatgcaagttgcacgaagccttgccttgccggtgtggctgcccgagaatccgccccggtccgctatcaaggaaagtgatcagcatgtcttacccgaagaagctaagtccggagttttccggggcactgagaaccttcagtttctgggtcgccaacggcaccttgggctatccgttgctggaaggcgtcgactaccgcagttgcctcggggacgagccgagcatgctggagatggcctatgccatttttgccaatgtgctggagctggacgagcagggcgtaccggtgaatgccaagtacgccgagcaccgggcggcgcagtacatccgccagtattgcgatccgggctaccaggcacggccggcgttcgaggactgggagcaggagctgtacggcccgcccgaccgggacgacagcaagccctggccagccggcgtggcctaggtttttcgacggctggatacaagcctcgggcactatggcttcgaaacgacgaaccccctcatcaaggattatgaatgtcgctgatttcgcaacgtgtagaactggcccgccagggtgccaacgacaaggtgatttgccgcatggcttcgggctgggcggtgatgggcgatgtgcagtttcttgagggctattgcctgttgttgccggacccggtggtggccagcttgaacgacctggatgcccaggcccgggccacctacctgctggacatggcccgcatcggcgatgcgctgctcgaggccaccggagcgctgcgcatcaactatgaaatcctcggcaactcggagcctgagttgcattgccatatctttccccgctacgcctcggagccggaaggcaagcgccggatgccggtgtggttctacgactggaagaccgcgccgccctatgtcgaggccgagcacggcccattgcgccagcagcttgcccggatactggctggccaattgtaggagcgagcttgctcgcgatggtctctaacgaaaacgccgcactcactgggggagcgcggcgtgcttgagtgcttcgcgagcaagctcgctcctacaaggggctgttgatcagcggtttgctgcgctccacttggctcgcgctgcgtgcagcttcttgtagctgtcgatcagccgcaggtggcggtccaggccttcgagctgcaggttggtcggggtcaggccgtagaagcgcacgctgccttgcaccgagccgatcaccgcgtccatccgttcgttgccgaacatgcgccgcaagttggcctcgtagtcgcccagctccagctcgtcgtccagctccacttccagcaccgcgttcagggcctggtagaacagcccgcgctcgacggtgttgtcgttgtattgcaggaacatctccaccagttccttggccgggtcgaactgccccagggccacgtggatcagcagcttcagctccaggatggtcaactggccccaggcggtgttgtcgtcgaactcgatgccgatcagggtggtgatgtcggtgtagtcgtccagttcgctttcttccaggcgctccaccagcgagcgcaggccgtcttcgtccaggctgtgcaggttgaggatgtcttcacggaagaacagcgccttgttggtgttgtcccagatcaggtcgtccaccgggtagatttccgagtagtccggcaccaggatgcggcaggccgtggcccccaggtgttcgtacaccgccatgtagacttccttgcccaggccctcgaggatgccgaacagggtcgcggcctcctcggcgttggccgccgctctttcaccgctgctgccctggctggtgaagtcccactcgacgaattcgaaatccgccttggcactgaagaaacgccacgacaccacgccgctggagtcgatgaagtgctcgacgaagttgttcggctccatcagcgcctggctttcgaaggtcggctgcggcaggtcgttcaaaccttcgaaactgcggccctggagcagttcggtgaggctgcgctccagcgccacctcgaagcttgggtgggcgccgaacgaggcgaacacgccgccggtgcgcgggttcatcagggtcacgcacatcaccgggaactcaccgcccagggacgcatccttgaccagcaccggaaagccttgttcttccaggccctggatgccggccagaatgctcgggtacttggccagcacttcctgaggcacatccggcagggccatctcgccttcgagaatctggcgcttgaccgcccgctcgaagatctccgacaggcactgcacctgggcttcggccagggtgttgccggcgctcatgccgttgctcaggtacaggttctcgatcaggttggaggggaaatacaccacctcgccgtcggactggcgcacataaggcaggcagcagataccgcgctcgatattgcccgagttggtgtcgtacagatgcgagccgcgcagctcgccatccgggttgtagatggccaggcagtgctcgtcgaggatctccttgggcagctcatccttgcgccccggctggaaccagcgctcgtccgggtaatgcacgaactcggcgttggcgatctcttcgccccagaactgatcgttgtagaagaagttgcagttcaggcgctcgatgaactcgcccagggccgaggccagggcgctttccttggtggcgcccttgccgttggtgaagcacatcggcgactgcgcatcgcggatatgcagcgaccagacgttgggcacgatattgcgccacgaagcgatttcgatcttcatccccaggcccgcgaggatcccggacatgttggcgatggtctgctccaggggcaggtccttgccggcaatgtaggtgccttcgctggagcctgattgcggcatcaacagcgcctgggcatcggcgtcgaggttttccacttcctcgatgatgaattcggggccggtctgcaccaccttcttcaccgtgcaacggtcgatggaacgcaggatgccctggcggtccttgtcgctgatgtccgccggcagctcgacctggatcttgaagatctggttgtagcggttttccggatcgacaatgttgttctgcgacaggcggatattgtcggtggggatattgcgggtctggcagtacagcttcacgaagtacgccgcgcacagcgccgaagaagccaggaaataatcgaacggacccggcgccgaaccatcgcccttgtagcggataggttggtcggccaccaccgtgaagtcgtcgaacttggcttcaagtcgaaggttgtcgagaaagttgaccttgatttccatgggggcaccggcatcacaaagcaaaaaatggcgggcattatccgggtttttgcgcgggaagtgttgtggttttcggcggacgggtggatcgccagggctggctgccggtcacagacaccggcacccggccccgggcatcactcaggcattcagactttgaggatgaaacgattggatatccgtgaatagcccaggggcaacaactcggcttcgtgctccttgatcagctcatgcagcgcgttggcggtttcggccaccggcctggcttgctccctggcctgttgcatgacctggcacgactgcctgaacaggctttcgtaggcgggcgagtccagggacaggcagccagggaagcgcaccgcctcaagcgctgcatcgaactggcgggcgtcgccactgtgttccaggtcgtgcagcagcttttgcagaaaggcactgcccaggttggagccgctgagccagacccgatgccacacgttccacaggtgaaagtcgctgaaggcttcataggagcaggagaccaactggtcattggtcgccaccgcgttcaggcaatggcgctcgacttcgatgaactgttcccgttgccagcggttgctgcgggcggcgtccagcaccttgggggccagcctgaggatggactcgaaggtggtgatcaaccccctggagaacagcgggtcgataaaccctgtggcttgcggcagcaggcagaagcgatccccgacattttgcacgctgcggtagttgatgcgcggcgcgtagatccactcccgggcattgaccgcatccttgaagtgttcgccgatggccgggtactttttcagcagtttgcgaaactcgatctccggcgcctcggtgggacggtacttggcgttgttgaactggaagccgatgctgcacaactgattggtgccctgcgggtggttgttgaaggggatgacccacaaccagccctcttcgaagatgtggtgcaaggtgctcttgaacagttcgatgggggaacgagtgcgggacaacggagccagggcatcttcgtagctcttcacattgagcatgtgggtgaagaatgagcaggtgtcggtcgccagcccttcggtggtgcgcaagcccagttggcgggaaagcggagagccctgggcagcagcgtcaatgatgaacgcggccttgacgggggcggcgttggacaatgccacctcgaccccgtcgtcgttgaggctgatcgactcgatcttgatgttctgtctggattcggcgccgtgtttcagggcaatcatcagggcgaaatagtcgatgtcctgccggaaaagatgggcttccggcaccttcagcggcggggccacaaggtggtcgggggacgacggcgcgttctcttgatgccaggcaaaactgaagcccagcttgatcccgcaggcgctcgaaccgacgtgctggatgatcttgtcggggtgcgagaggtaggcgatttcagggatgtcgaagcgctttgagagcaaacgcagcagaaaaccgctttccggtgtcgccgcttcgccgacggagaatcgtgggtgctgggccgagtcgaggatcagaacgttcagcccggacttcgcgaggacggcgccggtcagcgcgccggcgataccactaccgatgataatgacgtcgtactgattcatgatgtctggctcttgtcttgcaaggtggtcgctctggaaagcgctgaatgcattgcggtatcgaaatgcagggtggccagggtctgcggtatttccttttcaaacggcaagtcccagtaaggcgtgctgccgatcgagcgtttcaactcattgatgaacaggcgggtcttggccgggacaacgctgctgcgtgccctcagtgcgtagatcgctccctgttgcggatcggcctcgcaatattcgtcgagcagcgacaccagttggccgctggcgatctgttcatgcaccagccaggtgggcgcgtggatgattcctatgccctgaagggcgccgtccaccagcaactcggaactgttgaccgatagccggctgctgggcatgagcttcttgagcaggccatcgaccttgaattgccaggtcagtgagaagttgctgcaggtgctgtggatcaggcaggcatgttggtacaggtcttcaagagtttgcggacagccgtgttgccggatgtattccgggcttgcgcagagcacgcgccgcatatccgcaagtttcgttggaatcagattactgtccggcaatatgcccagcttgatacagatatcgacgttatcgatgctggggtcgagagctttgtcgttaagcgagaattcaattttcaaaccgggatgatggttcagtattctgctcaataaaggcgctacatggcgccgtccaaaggccacgggtgctgaaaccttgagaacgccaagggtcgatgcagtgtgttcttttacttctgatctagcatgcccaatgcatctcgagatgttctttgcatgttcgagaaagagcaggcctgtttcggtaagtatcaagcattttgtcgatcgattgaatagacaggtttcaagttctttttcgaggctattgattctgcgcatcaccgaagaaggtgggatgccaagtagtcttcctgtttcggagaaactacccgttttagttacggatatataaaggtctatgtcattgacaacgcctagcgccttcattcctaaatccttttagaatttttagtgtcttatttttgtaaaggcaaattacaaatagatcatgacagcctccttagtacggctaggctttttgtttgtcaagcagtgctggggccatgattagttggctttgggaaaattgattcataacatttcaggcgccacagctctacccggcgcagagcggaacaccgtccaagtgcaaggcttggtttgcactatctggatacaaaaatagttctagtggctaattgcgcagagcgccaaaactatgcttgggcgctcgaagatgactttctgaaggccgtcatataggcctgtggaacatcgttaagttgtgttggcttcgatgttgtcggggctgttttgcctttgcggatatgcaaaggccttttgcaaaaacggctattcacaagtgcattcttattgaggattgtagacgggttttcccaacccacgattgtcgaatgcaggagcgtacgtctatggacggagaggaagttaaagaaaagattcgtcgctacatcatggaagatttgatcgggccaagtgcaaaggaagatgagcttgatgaccagactccactactggagtggggcattctcaactcgatgaatatcgtcaagctcatggtctacatcagggatgaaatgggtgtctcgattccaagtacccatatcaccggaaaatattttaaagacctgaatgcaatatccaggactgtcgagcaactaaaggccgagtgcgcctaacagggagtgggcaatgagcgatcatgattatgatgtagtgattatcggtggcgggccggcgggttcgaccatggcctcctacctggcaaaagccggtgtcaaatgcgcggtgttcgaaaaagaactgttcgagcgcgagcatgttggcgagtcgctggtaccggccaccactccggtgctgctggaaatcggggtgatggaaaagatcgagaaagccaacttcccgaagaagttcggcgctgcctggacctcggcagattccggccccgaagacaagatgggcttccaggggctggaccacgatttccgttcggcggaaatcctcttcaacgagcgcaagcaggaaggggtcgatcgcgacttcacgttccacgtcgaccgcggcaagttcgaccgcattcttctggagcacgcaggttcgctgggggccaaggtcttccagggcgtggagatcgctgacgtcgagtttctcagcccgggcaatgtcattgtcaatgccaagctgggcaagcgcagcgtggagatcaaggccaagatggtggtggatgccagcggtcgcaacgtgctgctgggccgccggctgggcttgcgagaaaaggacccggtcttcaaccagttcgcgattcactcctggttcgacaacttcgaccgcaagtcggcgacgcaaagcccggacaaggtcgactacatcttcattcacttcctgccgatgaccaatacctgggtctggcagatcccgatcaccgaaaccattaccagcgtgggcgtggttacgcagaagcagaactacaccaactccgacctcacctatgaagagttcttctgggaagcggtgaagacccgggaaaacctgcatgacgcgctgaaggcatcggagcaggtccgcccgttcaagaaagaggcggactacagctacggcatgaaagaagtctgtggcgacagcttcgtgctgatcggcgatgccgcacggttcgtcgacccgatcttctccagcggcgtcagcgttgcactcaacagtgcgcgcatcgccagcggcgacatcatcgaggcggtgaagaacaacgactttagcaagtccagtttcactcactacgaaggcatgatcaggaatggcatcaagaactggtatgagttcatcacgctctattaccgcctgaacatcctcttcaccgcgttcgttcaagacccacgctaccgcctggacatcctgcaattgctgcaaggggacgtctacagcggcaagcgcctggaagtgctggacaagatgcgcgaaatcatcgctgcggttgaaagcgacccggaacacctctggcacaagtacctgggcgacatgcaggttcctaccgccaaacccgcgttctaaacactaaacacccagtcgaaggagatgactgaatggatgctcgtgcgcccatggattttgaacccattgcaatcatcgggagtggatgccgctttgccaaaggtgcatcgacccccgaggcattctgggagctccttcgtgctgggaccgattttgtcggaccggttcccgcagagcgctgggacacagcggcgatctacgatgagagcgctgctgaaaccggtacgacctacagcaaggtgggggcgttcctcgagcacatcgatcgcttcgatgcgcattactttggcatctcggcctctgaagccaaggaaatggacccccagcagcgcctgctgctggaggtcgcctgtgaaagcgtggcccgtgccggcctgacccgcgagcagctcaaaggcagcaggaccgcggtctatgtcggcatgctgggcatggattacctggcactgcactcccgcgaggccggcatcgaacagatcaacccctactatgccgccggcaaggaattcagcttcgccgccgggcgtattgcctaccacctgggtgttcatgggccggcaatgaccgtgaccactgcatgctcttcttccctggtggccatgcacctggcctgtcgcgccttgcaggcaggggaggcggacatggccctggccggaggggtgaacctgatgctggcgccggacctgacgatctacatgagccagatcagggcgatctcgcccagtggtcgctgccgggtattcgatgcggcggccgacgggatcgtccggggcgaaggctgcggggtcacggtgctcaagcgcctggcagatgccctacgcgatggcgatccgatccaggcggtgatcaggggctcggcgatcaaccaggatggcgccagtgccggccagaccgtgcccaacgccaatgcccaggctgcagtgatcagccaggccctgaaagtggccggcttgagcgtggacgacatcgactatgtcgaggcccacggcaccggtacgcccctgggcgatccgatcgaactctcttcgctggacagtgccttccaggggcgtgagcggccgctgtgggtgggctcggtcaaggccaacatgggccatctggatgcggccgccggaatggccagcgtgatcaagaccatgatggttctcaagcacgccgaggtgcctgcgcaactgcatcttgcacagttgaacccactggtggactggaagcgttcccggctggcggtgcccacggcgatcgagtcgttgcccgaccggccgcgcctggctggaatcagtggtttcgggctcagtggcaccaacgtccacatgattctcgaagatgccagcgtctatcggcaggcacagccgcagcaggaacgatcggcacagggccggccctgggtcctgccggtatcggccaggtccgcgcaggccgtggtggagcaggccagggcctatgccgttcatctgccgcagcaggacgatgggcaattgcaagcctttgtcgccagcgccattcatcgtcgtgatcactttccctaccggtcggccgtggtgggtgcgaacgccgggcaattgaagagccagctggagcagctgccggccccgaccttggcctgcacgacggacgaagaggatcggcgcgggcccgtgctggtgtttaccgggcagggcgcccagtgggtgggaatgggccgcgacctgcttgaacgggagccggccttcctggcgatgatccggcgttgcgaccaggcgctggcccagtgggcaagctggtcggtggaggccgagctgcgcagcgacgccagtgggtcccgcctgcacctgaccgaatttgcccagccctgcatctttgccatccaggtggcgatcagtgaatgcctgcgccagtggggtgtgatcccggcggcggtggtcggccactccatgggggaagtggccgcggcctattgcgccggagcactggacctggagtcggcggtgcgggtcatccaccatcgggcccaggccatgaaggacaccctggggcaggggcggatgctggtggtgggcttgcccgcgccgacgctccagtcgcgtctggcgaacaacccgcaactggaactgtcggtggtcaacagtcgaaacagctgtgtggtgtccggtagcccgcaggctgtacaggctctggatcagcaactgcgtgacgaaggcatcttcacttacctgatgccggcggaatatgccttccactcctgccagatggatgagtgcctgacgcagatccgcgcggggctggaggatttgccggtggttgccgcgcacacgccctggatttccaccagcgcgatgcccgaggagccgatcctggcagatgccgactactgggcgaggaatgcccgtggcatcgtgcgcttcgatcgcgccatcgaacagttgatcgagcaggggcaccggctgttcgtcgagatcggcccgcacaccgtgttggcggcgtcgatcaaccaggccctggccgacaagggaacccagggactggtgtgcggcgccttgcacaagcagggcgacgccgccctggagctggccagtattgttgcccgcctgtacgagtggggcgcaggtcccgactggcaagccttccaacccagggaggcggcgctggagctgccggcctatccctggcagcaggagcgtttctggtttgccccggcgccgcggccgcagccggcagggctcgtgtcccagctgcgggcgcaggtactggtgtatgacgcccagggcaatctctgcgcccaggccaacgatgtggcgctgagcgtgccgcagctggcacaagtcgctgtgccggcaccggccaaggtgtccgcagcggcgcagccggtgggggatgtgcgggcgcagattggtgcactgctgacgcagatcatcggcgtggcctgtgccgacccggacccggatcgaggcttcttcgaactgggcctgagctcgatttccctggttgaattcaagcgcatgctggagcgccagttcgccctcaagctgtcggcgaccgtgggcttcgactatcccaccatcaaccggctgggccagtacctggaagggctgctgtcccgggagccggccagcaccccggtaacggtcgatgccggggcgaccgacgccgcggggagcgtcgccgtggtggccatggcgtgccggtttccccaggccgacagccccgaggcactctggaagctgatgctggaacagacggacaccgtggggccggtaccgccgtcccggctcgccggcgctaagccggaggaaaccttcccgcggtttgccagccttatccagcgccccgaagggttcgacgaagcgttcttccgcatttcccccaaggaagcccggagcatggacccccagcaacggctgttgctgatggtggcctgggaggctctggaacgggccggtatccctcaggagaagctgctggaacagagggtcggggtgtttgtcggggccaactcccacgactacgaaacccgggtcctgggtagcgcgcaaggcgtggatgcccactacggcactggcagttcgttttcggcgatctgcgggcgcctctcgcattttctcggcgtgcgtgggccgagcctgacggtggacaccgcatgctcgtcatcgctcacggccatccatctggcgtgcaacagcttgcgtgccgcggagtgcgacatcgcgattgtgggtggggtgaatgtcatcgcctcggcgtcgatctttcaatccatggggcaggccggcgcattggccccggacggcatcagcaaggccttcgacgacagtgccgacggttacggtcgaggggagggctgcggcgtggtcattctcaagcgccaggcccaggccgagcgggagcgtgacccgattgtcgcgacgattctcggttcggcggtcaatcacgacggtgcctgtgccgggctgacagtgcccaatggtccggcgcaagaggcgctgatcagcgaagcactggccaacgccggcgtgcatccggggcaagtcagctacgtggaggcccatggcaccggcacggtgctgggtgaccccatcgagctcaacgccctgcacaacgcctatcgccaggccagccccgacagtccgccgctgacggtggcctcggtcaaggccaatatcgggcacctcgaggcggcagcgggcatcgcttcgctgatcaaggcctgcctggtggtggagcacggccggattgccccgcaagcccatctgcagcgggccaacacccgtgtcgactgggcggccatgaacctcaagctggcgcatcaggccatggactggccgggccggccggagtcgcgggtggccggggtcagtgcctttggtttcaccgggaccaacgtgcatgtgctgctcaagggctatacggcgcccgcgacagcgcctttgccacccgccacagcaccagtggccttgtgcctgtccgcggccaccccggcggcattggcggagctggcccagcgctatgtgtccttcctgggcgctaccgagcactgcccacagaccatctgctacaacgcgctgatgcggcgcacggcgttcaaggaacgcctggtcgtccacggccaggattgccgcgagctggctcaggcactacaggcctggctggccggtagccccatcgccaatgaccgcaaacccgcggccggcgaaccctgggcaaccctggccgaggcctttggccgcggcgcccagtcgccgggccccgagcgcttgccggacggctgtcaggccatcgggttgcccacctatccctggcagctcaacgactactggatcgatgctggccagccggctacggcggtgcagcccgcccgggcagcctcgggccatccttgcctgcaggggctggtacggccggccgggcagctctggtactggtcgggggctctcgctccccaggccgggcattacgacccgctgggcgagcaggggtacagggtcaagactcacctgttgctggatgccgtgctccaggcggtgcgggaaacaccacggggcgtgcagcagatccgcgacttgcagattgcccagctgcgcctgcgcggcgaacagcacctcacttcgcacctgagcatccacctcacccaggcgcctgacgcctgcttcgaactggccttgcaaggggccggcgacgagcgccgccaggtctgcatgagcggaaccctggttgactgtgcagcgcagttgcaggaggaaaccctgtgcggggtgtcgatgctcgatgagccttcaccgccggcgccggacgttggcctgtgcccatggtccgggtgtgcggccacgggcgggcagcgggccttgtaccgttttgcccactccctgacggcggccgagcgccagacccagttgctggccagtgtggtcgagctgtttgaaggcgcccacgccgccgcgctggtcgggttctccgggctccaggtatgggcagacctgccggcgcaggtatggattgtgctggccggccatgacgccgacaaacccgacagcctgcaggtagtggatgcccggggttgccagctggcgctgttcgaaggtccgcagtttggccatcctggttcgtggtacttgcccgacctccagaccgcacccctcgacctgccgatgatcgctcgccaatggcaggactatccgatgccgggcgagggcgctcggcaacgcgagggctactggatggtgctggcctggagcactgccgaggtccagccactggctgcggcgtttgcggctgaacagcgtccggttgaagtcatcgagctgcacgccgggcaacaacctctggcccgcaaactgtcctcggcgttgcgcggggcagtggccgatccgtcctgcctgggggtgatcgttgccggcgttgaggctcaggaggccgacggcctcggtatatccctggtggcctctgcggcgctggtacaggccttcgccggtgcgattgccagcgtcggaacaccggcaaagccggtctggttcgcccttcatgccagtgatgctgcgtcgccggccatggccgctgtccaggccacctggcagggcgccgcgcatatcttcgccctggagcatcctgcctggtggggcggtctggtgactttgcagggcagcgacagaagaagctacgccagcctctgccggctgttgcacggccaacctggccatgatcactttgcgatcagcggcgcccgggtcgaggtgcaatacctggtggaggatcaagccgacccgctacagcgtctggagccgccggcgctgaacggaaccgttgtgctgcatgccgtcccgggatctgacctggagactgtgctgacggcgctcgggcagcggggtgtgcagcgtgtgctgctgctctgcgaggcccccgggcaactgcacatgcctgagcggatgcccgaagcgatggcgatcagcagcctctcggacctgagccgggaaaacctggctgacaccttcgcgacgctgcgagcgcaagaccgcatcgccggctttatccacctcgatcttgactggcgcacggttgcgctcaaggagccagagtttgtcgtgcgaatgcaggaaggcgttcggccgcttgaggtcttgcagcaggttcaccagctgatcgatgaccctgaagcgttcttcctgatcctgggcagcgtgtcttccctgcttggcggggccggcttcgcccgctcggcgattgccgatgcatatgccttgtgggtgcatgcacagcgccggcgccaggggttgaactgccagctgctgcacctgacgcagagcgaacaggagcttgagcaggacgccgcagcccgcacggccatgcaaggcagcggcctgcagcctttgcagcgctcgcagatagtgcaggccatagcccgcgtgctgggtggccagggccagtgcgggctgctcaatgtcgactggcaacaactcaaagggctgtacctgagcgtgctgccctggcccttgctcgaacacctgggtgcagcggatagcgcagcggatcagcgtctggccgagctgattggcctgccaccgctgcagcagcgccgggccatgcaggcgctggtctgcgaggtagtggggcaggtgttcggcgttgccgatggcctggagctcgatgtcaggaagggcttcttcgacatgggcatgtcctcggtcatgtcgctggacctgcgctcacggctgggccgggcgttgagcatcgacctgccttcgaccttcggcttcgaatacacctccatcgaacaggtcacggactacctcatgggccagctcctggcgcctgaaacccgggagccggtggcggcgcccgaacctgtgtcgccagcgtcgcggcatcaggacctgcatgaactgtcacgggccgagctgattggtgctctcgaagacgagctgcgcgatatcgccaattactagccctggggctggatgacaactgccagagaatcactgatcgaggcctttatggataacgatgtccgcgatgtaagcaaggaacagctgcaagagagtcttgctcaggcaatcactaccatccgtgcgctcaaggaaaaggtggccggcaagagctcggcgcctgtcgaaccgatcgccgtagtgggcctggggtgccggctaccgggcagtgccgacacaccgaagcggctgtggagcctgctgaaacatgccaccgatgcggtgggcgacatgcccagcgaccgtctgtacggcaccgactattaccatcctgatccccaggcacccggcaaggcctacgtcatgcgcggtggcttcatcgagggggtggatcagttcgacccgggcttcttcggcatttcgcccaaggaagccgaaggcatggacccccagcagcgcctggccctggaggttgcctgggaggccctggagaacgccgcgatcgcccccgacagcctgcatggcaagaagctcggcgtgttcatgggggtcagtaccaatgattacgtgcgcctgcgccagcagttgggcgcggtcgaggacgtcaacgcctaccagttctatggcgaaaccagcttcgtggccgggcgcattgcctacaccctgggctccaggggcccggcggtggtgctcgacacctcctgctcctcatccctggtggccctgcaccaggcctgcaacagcctgcgcagccgcgagagcgagctggcgctggccggtggggtcaacctgatcctgtcgccctacggtttcatcctggtcagcaagctgcgggccgtggcccccgatggccgctgcaagaccttcgacgcggcggccgatggctacgggcgcgccgaaggctgcgtgatccttgcgctcaagcggctgagcgatgcggtacgcgaccaggacccggtgctggccgtgatcgagggtagtgcggtcaacaacgacggcgccagcagcggcatcaccgtgcccaacatccacgcccaggaagaggtgatcaggctggcgctcggccaggccgggctccagggcagcgaggtcgactatgtcgaggcccatggcaccggcaccgcgctgggcgacccgatcgaactgcacgccctgcatgcggtactgggcaagcagcggcccgtcgatgcaccgctgctggtgggctcgatcaaggccaacatggggcatctcgaaccggtcgccggggtgaccgggctggccaaggtcctgctgtgcctgcaacaagaggccctggtgccccaggtgcacttcaacacgcccaacccgcggatcgaatgggatcgcctggccctgaaggtggtcaccgaatccacgccctggccacgccagggcaaggcgcggcacgccggtgtcagttcgtttggtgtcaccggcaccaacgcccatgtgctagtgggcgacgcgccgctgcgcgaacgtgcccaggggcgcgacaacccctggcagctgatcactctgtcggccaagggcgagacgccccggcgccagattgccggacgctatgaacgttttatcgccgacaacagccagctcgaactcaaggacctgtgctacacggcgaacgtcgggcgggcgcactttggccatcgtttcgcggccgtggccgatagccgtgaggggctgcgcgagcaactggcagcctatgcgtcacgcaaggtcgtggggcatgtattcgaagggcgctgccagggagcggcggcgccgctggtgatgctctttccggggcagggctgccagtaccgggcaatggcccaggcactgtatgacagcgaaccattcttcaaggcgcagatcgatgaatgccgcgccctgttgcagccgctgatggacgtggacctgctgaccctggtgctggacgcgggtgcggccagtgacagctacctgcaacagacccgctatgcccagccggcgatattcgcggtcgaatatgccctggcgcggttgtggatgcattggggggtcgctgccgatgcgctgttcggacacagtttcggcgagatcagtgcaatctgtgtagccggggcggtatccctggctgacgcgctgcgtatggtggaggcgcgtgggcgcctggcccagcaactgatgacggccggcggcgcgatgtacgcactgggcatgagcgaggcgcaactgctggagctgctcaaggaccggcccggcagcgcgatcgaactggcggcggtcaacagcccgcaggacgtggtggtggccgggccgcaagccgaggtccaggcgctggccgaagcggcgctcgccagcggttgcaaggtcaagaagcttgcggtttcccatgccttccataccgcagccaccgagccgatgcttgaagcgttccgccagacggtggcgcagatcaccttcagcgagccgcgcttgccggtcatcagcagtgtcactggccgggtgcatacgctctccagcctgagctcgcccgattactggtgtacgcacacccgccaggccgtgaggttctgcgaaggggtcaacaccctgatcgcagagctgggggtgaaaaccttccttgaagtggcctccgatgctgtgttgacgccgctgatcggccgtcaccccctggcggacggcagcctggtcctggccagcctgcgccgggccggcgatccgtccagggacctgcgcctggccgccgcgcagctctacgtgggcggccataacctcgactgggcccggctgcacgagcatgacggggccttgcgccaggccttgcccgggtatgcgtttcagcgccagcgctactggttcgacaacgccagcggctcgcccctgcaacaggtcgctgcgggagcggtcggccggctgctggggcattcggtcaatgcacccactccggcattcgagtcgacgctcgatagcgcgctcctgcaggttgtgggcggcgagatccgcgatgacctggcgctgctgcgtcctgaccggttgctggcagccctgagcgatgagctggccggccacttgcagctggacacctatggggtgagcctcgccagcatcaccccggccctggcgttccatgtcgatgaccagttgcatctgttcaccgaactcaagccgctgtccggggcggcctgggaggtcaactgttcggccctgagcgcagcggccaaggttgccggtgccgattggcagccggtcctgtcgctgaccctggaaggcctgccggcggctgtccggggcggcctgccggacccgggccatgccgccacccaggacgcaggctttgtctaccacatgcagttgccggcagaacccgatccgcacggcaatcaccttgggcaggtcctcgagtacctggggcaggcggtgccgggcgatgcgccgggctcgataagcggcatccgccgctgggtcgccagccggtccgcttcgccccgggcacaaagcctggtgattgccacgagccaggcacatccgcagcagttcgactgcgccttgtacgatgcgcacggccagtgtgtaggcagcctggaagggctgaccctggctgccgccccgagtgaggaggcgctgcgtggcatgctttaccagcccgacgtgctctacagcctggattggctcgaacgccctcgccagcgcgccgaagtgccgcagcaggaccgcttgttcaccctggtcagccgctcgccggagaccgccgctcccctggtcgaacacctgcagcgtggcgggcatcgtgcccgggtcctgtgcccgcagcagttgctcgatagcgcgcggagggtgctggcccaggacccgagccaggcattgaccgatgaaggcgaggccttggccgccgatatcatcgtgctcgatggcaaagacatcgaggatgccggctcgaccacgctgcagagcctgtcgagcctgcaggcgacactgttccacccgttgctggagatggtccaggcgctcatcgaactgggcccgcgcggtggccggttgtggctggtgacccaaggggcaaaccctgttggcctggatcgcgaccagcccctgcaagtggcgaccgggccgttgtgggggctgggcaagacgctcgccctggagcaccccgaacactggcgcgggctgatcgacctggcccccgacgatcctcactgggcaagggcgctggccgaagaagtcagcgactccgatggcgacgacaagatctgcctgcgcccgggcaagcgttatgtccagcgcctgaaccatttcagcgctgcgcagttgccagcacaggcctatgcaccttgcccgcagggcagttacctgattaccggcggcatgggtggaattggcctggccatggcccagtggcttctggataaaggcgcgggcgctgtgctgatcaccggccggcggccactggaggatgtcgccacgggcctggagcgcttcggcgctgcggcatcgcgggtgagttacgtccaggccgacatcacctcgccccaggacatgcagcggctgttctgcggtctggaggcgtcgggcgcgagcctgaagggcatctttcatgccgccgggatttcaattccgcaggatctcaaggacgtcgaccgtgacagtttcgaccaggtgatgcggcccaaggtcgagggcacctggttgttgcacgaactgtccctggggctggacctggacttctttgtcctgtgttcgagcatcgccagtgtctggggttcgcagcatgtcgccagctacgcagccgccaatcagttcctcgacagcctggcgtggcagcgtcgggccatgggcctgagtgccctggtgatcgactgggggctgtgggccggcggcagtcacctgttcgacgagcaggtgctgaatttcctcaccagcgttggcttgaaacagatcgccccggtacagaacgtcggcctgctgtcgcggattctggccagcgagctgccccagatggtggtgtcaggggtggactggaatcgcttcaagccactgctcgaatcccgcggcccgcaaccgctgctgcagtacatccgcagccaggctccgaccgccagggccggcgacagcagcaacgtggagatcctgcagcaactggcgggcgccgatgaagccgctgccctggcattgctggatgactacgtgtgggagcagtacgcgcagttgctcggggtcaagaccgaacaggtgcgggccaagctcgaggatggcggcagcctgatggactacggcctggactcgctgctggtgatggacatggtcgcccgctgccggcgcgatctgaagctggagatcaaggcccgcgagtttcttgagtgtccgggcctgatgtggccggacttcctggcccgttcgataaaggaacagggctgcgtggccgaggcctgacgggccgcgggcagtgcaacccagacccatgatctgtgattgaggtggttatgaacgatgtgcagtctggcaaggcgccagagcattacgacattctcttggcgggcaacagcatcagcgtgatcatgctcgccgcctgcctggcccggaacaaggtccgggtcggtttgttgcgcaaccggcagatgccccccgaccttaccggtgaggcgacgattccctatacctcgatgattttcgagctgattgccgaccgctatggcgtgccggaaataaagaatatcgcccgcacccgggatatccagcagaaggtgatgccgtcttccggggtcaagaagaacctcgggttcatctatcaccagcgcagccgggcggtggacctgggccaggcgctgcaattcaacgtgccctccgagcatggcgagaaccatctgttcaggcccgatatcgatgcctatctgctggcggcggccatcggttatggcgcgcagctggtggagatcgataacagcccagaggtgctggtcgaggacagcggggtcaaggtagctacggcactggggcgctgggtcactgccgatttcatggttgatggcagccagggcggccaggtgctggcgcggcaggctggcctggtcagccaggcttcgacgcagaagacccggaccctggaattctccactcatatgctcggggtggtgccgttcgatgagtgcgtgcagggcgattttcccggccagtggcatggcggcactctgcatcacgtgttcgatgggggctgggtgggggtcatcccgttcaacaaccatcagcactcgcgcaaccctttggtcagcgtgctggtttcactgcgtgaggacctctgcccgagcatggacggcgaccaggtcctggccggcctgatcgagctgtaccccggcctggggcggcacctgtccggcgcccggcgggtgcgcgagtgggtgctgcgccagccgccccggcaggtctatcgcacggcgctcgaacgccgctgcctgatgttcgacgagggcgccgcgagcaacgatctgttgttctcgcgcaagctgtccaatgctgcggaactggttctggccctggcgcaccggctgatcaaggcggcgcacagcggtgactaccgcagcccggccctgaatgattttgtcctgacccaggacagcatcatcagcttgagtgaccggatcgccttagcggcttatgtgtcgtttcgcgaccccgagttgtggaatgccttcgcccgtgtctggctgctgcagtcgattgccgccaccatcaccgcgcgcaagatcaacgatgcctttgccaaggacctggacccgcgagtgttcgatgaaatcgaccagctcgcagaggacggtttctggatgcctctgtatcgggggtacaaggatattctcaacactacgctgggcctttgtgatgacgtcaaaagcgccaaggtctctgctgcgcacgcggcgagcagcatctttgcggagcttgccaacgccagttttgttccgcctatttttgattttgctaatcctcacgctcgtgtctatcaactgaccaccttgagaaagctcaaggcgctctggtggggcctgatgcaagtgccctcagaggtcggacggctgattttctatcgatccttcagaaaaccttccctgcgcaaggagagttgaaatggacttcaactacgacgatacccagaaaaaacatgcggccatgatcgcccaggtgtgtgccgagcaattggcggcctgtggcaatgaacactcgcggtatttcactgcccggcaatgggcgatctgcggcgaggccggattgctggggctgtcgattccccgggaatacggtggccagggcctgggtgcactgtcgacggccattgccatgcacgcctttggcctgggttgcacagacatgggcctggtgttcgcggcggcggcccaccagtttgcctgtgcgatgccgatcgtcgagttcgcaacagcggaaaccaaacgcgatgtgttgcccaaactcgccagcggtgaattcatcggctccaacgcgatcaccgaacccgaggccggctccgactccagcaatttgaagagccgtgcctggccccaggccgatggcagttatcgccttgacggccacaagagctttgccggcaatgcgccgattgccgacatcttcgtgacctatgccaccacccagcccgagtacggtgccctgggggtcagcggttttatcgtccaccgcagcagtgcggggctcagggtcagtgagcccctggacaaggtatgcctgagaagctgccccgcgggtgaagtgttctttgacgattgcagggttcctgaggtcaaccgcctgggtgaggaggggcagggccggcaggtgttccagagctccatgggctgggagcgtgcctgcctgttcgcagcgttcctggggatgatggagcggcaactggaacagaccatcgagcatgcgcgcacccggcgccagtttggcaagccgattggcgacaaccaggcggtttcgcaccgtatcgcgcaaatgaagctgcgcctggagtcggcgcggttgctgctgttccgggcgtgctggggcatggaccagggcgatccggggcagctcaacattgccctgtcgaaactggccatcagtgaaggggcgctggcatctagcatcgatgcggtgaggattttcggcggccggggctgcctggagtctttcgggatcgaggcgatgctgcgcgactccatcggcactacgatcttttccggcaccagcgacatgcagcacgagatcattgcccgggagctgaagctatgaagctgctccatgaacggatgatgcacagccttgcccgctacccgcggcagacggcggtggtggatgagcaggatgccttgagctacgaggcgctggagctcaggatccgggaattcgtggcaatgctctgtgccctgggggtcggccaggggcagcggatcctgctctgggcgcacaagtcggtggacctggtggcggtcatgcaggcggccctgcggctgggggtggtgtatgtgccggtggaccctctgagcccggtgtcgcgcctggaaaagatcgccggggattcccaggccgtgctggtcctctgcaccgcggcacgcctggaagaactcgccggctccgcgcttgcccaggtgcgcagcgtggtcctggacgacccggccagcgccggctactggcgcaacatcgataccggctccagcgtagtgccgacgctggccatccagccggacgatctggcctacatcctctacacctccgggtccaccggcgtgcccaaaggggttgcgctcagccacggcaatgccctggccttcgtcgactgggcgtgcgagcgctattgcttccagcctggcgagcgtttcgccaaccatgcccccctgcatttcgacctgtcggtcctggacatctactgcgcgctcaatgtgggcgcgacggtgtgcctggttcccgagtcgatcgcgttctcgccgcggctgctgaccgacttcatccgccagcacgaaatcagcatctggtactcggtgccctcggtactcatgatgatgatgcaagacggcgacttgctcagcgatatccaggacaccctgcgggtactgttgttcgccggcgagccttttccggtcaagcacctgcgtgacctgcgcgcggcctatgccgatgtgcgtctggccaatctcttcggccccacggaaaccaatgtgtgcaccgcattcgaggtcggcgccatcgatcccgagcgcgtgctcccggtgcccatcggcacggccgcctccggcaaccaggtgtgggcgcaaaagcctgatggcagccgctgcgcagtgggtgaagaaggtgagctggtggtgcaggggcctacggtcatgctgggctatttcgccaagccggctcaggaggggccctacaagaccggcgatatggtcaggcagcggcctgacggcaactacgaatacctggggcgtcgtgacgacatgctcaaggtgcgtggcaaccggatcgagcgcggggaagtcgaagccgcgctgctggcccatccccaggtcagcgaggccgccgtgctggtggtcggggaggggatgaacgcgcagttgtggggcgtgctggtcgctcacacccgggacgctctttcgctgatcgacctcaagcgccactgcgcccagcgcctgcctcgctacatgatcatcgacaaggtgctgtgcctggacgcactgccacgcaacgccaatggcaaggtcgatcgcttcgccctggccaggcaggtggagggctgacccatgggaactccatcacgcgtggccaaggacgcctggtttccctatgccagccggcctcggggcaagatgcgcctgttcacctttccctatgccgggagtggggcctcggtctttcatcgctggttcctgcccctgtacgaccaggtcgacctgtatgccttgcagttgccgggccgcgagggtcgcagccaggaggcctgctacagcgacatgcaggcggcggccaatgacgtggccgactgcctggagcagtttggcgatgacatcccctgctgtttcttcggccacagcatgggcgccttgctggcctttgcggtcgccggtgtgctgcaggagcggcggttgccgatgccgcagcaactgatgctctcgggcatggttgcgccccatgtgcggcaacgggtagcaccgctgcaccagctgccggcagagcaggccatcgccgcgctgcaggccatggggggcgtgccggcggtggtgctggccgaggaggacctgatgcagatgtacctgcccatcattcaggccgacatacagatggtctactcctacgccggtgcccagccgcagccgctggacacccgcatgatctgcctgagcggcgcccaggacctgattgcgccgcctgcgcagatgcagcagtggcggcgctacaccctgggcggcttcgagcagttcgtgtttgccggtggccatttctttctcgacgcccaggtcatgtcgcgggtcaaaacagtgctgggcagcgccctgcacaatcagcctggcagtctggcggtgtgagccgactattgggcaatggccgcgaggccggacctggcgaacagcttgaagtgggctttcagggcgttggcgtcgtgttgcaggacggcttgcagcgggctgggctgctggcgatctatcaccgccagcatcaggcagggcgcgatgatgctgatggtgcaacgcgccagcgccgggtcaccgatcggaaagccggtgatctcgctgatgagcgcttccagcagacggaatttgggcatcacttcttcttgcacaaggctggtgaaatggacagtgggcgccagtatttcccgtgcacagactttgctttgccagctctgctcatcgagcacccgggtgacgatggcatcgatgaagctatcgagcttgatcagcgccggctcttcgctttgcgccagctgcgacagggcttcgaagctcaccagctgcttgtgtccctcgatcagcaccgccttgtacagggcttcccggctaccgaagtggtagttgatggcggcgaggtcggcgccggcttcttcacagatgagcttgctcgcggtgttggcatagccatgctgggcaaacaggcgggcagccacttcgagaatgcggattttagtgtgttcgccgtcgcttctgctggttctgcgtcgcgtgttcatgatgttcctcgtggtctgagcggggggttgcttcatggccagtatctgttattttctaatttaaattcaaattgaattttaattaggccttggcaaacaaaggactcatggatgaagaagcagttgattgtaggggttgccgtactgctggtggccacagcgggtgtttcctggtttctccttcgccccgagaagcagaacgaccacctcaagctccatggcaacgtcgacattcgccaggtgtcactggcgttcgacggcagtgagcgaatcgccgcgctgtatgccgaagagggcgacctggtgcagccaggccaggtcctggcggaactcgacacacgcacactgcgcctggaaataaatcgctccaaggccaggatcggcgcccaggaacaggcgctgctgcgcttgaaaaatggcacacggcctcaagaggtcgagcagtccaaggcacgttttgatgccgcccaggcgcaaatgcagctggctcaactgcacatgcagcgcctgcgcaggatcgccgacgacacccagggcaagggcgtcagccagcaacgcctggaccaggcagccgcccgcttgcaagtggccaaggcccagtcgcaggagcagcgcgaatcctggaccctggccaagatcggcccacgcaatgaagacatcgcccaggccacggccgacctgcaagcctccagggccgacctggacctgctggaacattacctggcgcgcgctcagctcaaggcgccgacccaggcccggatccgcacgcgcctgctggagccgggggatatggcctcgccccagcgcccggtgtttgccctggccctgaccgaccccaagtgggtacgggcctatgtcaacgaacgccagttgggccacatacgtgcggaccagatggcgcgggtctacaccgacagctttcctgaccaggccatcgacggcaaagtcggctacatctcctcggttgccgaattcacccccaagtcggtggaaacggaagacctgcgcaccagcctggtctacgagatccgggtgctggtcaaggaccctgacgatcgcctgcggctcggtatgccggcgaccgtctaccttgaccaggcaccggtggccggggccacgccatgagtgcgccagcggtaacggttcaggctgttgaactggtcaagcgctttcgcagtggaacgcgagaacagttggctctggagcaggtttctctggaggtgcccagtggcaagttgagtgccctggtggggccggacggggccggaaaaaccaccctgttgcggatgattgccggcttgctcaaggccgacgaagggctgttgaaggtgctggggctggacgtcgccgcagatccgcagcaggtgcaggacctcatcagctacatgccccagaagttcggcctgtacgaagacctgacgatccaggaaaacctcgaactctacgccgacctgcatggcgtgcgcgcccagcagcgcgaggagcgcttttcccggttgctgcagatgatggacctgacgcgttttcgcgaccggctggccggacagctttccggcggcatgaaacagaaactggggctggcctgtaccctggtccgttcaccgcaactgttgctgctggacgagcccaccgtgggggtggatccgctgtcgcggcgggaactgtggagcatcatcgaacaactgatcgagcaggaaaacctcacggtgctgatcagcactgcctacatggacgaggcccagcgttgcgcccaggtctttgtgctataccaggggcggctgatcgcccaggggccaccggcgcagttgtgccatctggcgcagggcctgtgctatggcgtcggccccgaggccggcaccccggccaggctgttgcaggcgcgcttgcttgacgacaatgaacacatcatcgatgccgtgcccgaaggcggcatggttcgctttatccgtagcgcccaggtgcctctgcaggccattccatccctggctggccaggcctctccccaggcgctggaggcccgcctcgaagatggcttcatggtgttgctccggcaacaggttggcagcgttgcaagcctggacgagccggcgcaggcgcccgccagctacggccagggcgacgctgcctctgggcgtgcggtggtcatcgaggtccgcgacctggtgcgcaagttcggggacttcaccgcggttgccagcacctcgttcgaggtgttgaagggcgagatattcggcttgctgggccccaacggagccggcaagaccaccacgtttcgcatgctctgcggtttgcttcccgcctccagcgggtaccttgaggtggctggcgtcaacctgcgcaccgcacgggcggcggcgcggcgcaagatcggctacgtctcgcagaagttcgccctgtacgcgaacctgtcggccctggagaacctgcgcttcttcggcggcgcctatggcctgcacggcgcgaagctgaaagcgcgggtggcgttgatgctcgaacagttcgacctcgacgaacacaagcacctgcgcagcggcgagctgcccggtggctacaagcagcgcctggccatggcggtcgcgctgctgcacgagccacagatcctgtttctcgacgaacccaccagcggtatcgaccccttggcccgccgggcgttctggcgccgtatcaccgccctggcgcaaagcggcacgacgatcgtgatcaccacccacttcatggaagaggccgagtactgcgaccgtatcgtcatccaggatgccggacggctgctggccctgggcactccggaacaggtacgccaccagggcagcgagtcgcttgccgacatgaacagcgccttcattgccgtggtcgagcgtgctcgggcccaggccagcgccccggtgcagtgaggaatgaccatgactgactccaatcgatccgcctcagggttcaggcgcaggctcattgcgctcacccgaaaggagttgcgccaattgatgcgtgacaagagcaacctggcgataggcattgtcctgcccatcgtactgatcctgatatttggctacggcctgtccctggacattcgcaacacgccgctggccgtggtcctggaagacagctcccccagcgcccgcagcgtggtggcgaagcttgcgagttcggactacttctcggtggtccaggtgaccagcatggccgaggcccgagcgctgatggacaagcgtcgggtcgacggcatcgtgcgcgtgccggcggatttttccaggcgcctcgaacagcaggatgcgcgcttgcaattgctcctgcatggcgccgatgccaacagcgcggcgacccttggacgctatgtcaggggcgcgctgaatgtctggcaacagcagcagttcgaccgctcccggcagccccagtcggccggtcgggtgctggtcgtcgaacgcatgtggttcaacgccgccaacagcagtacctggtacctggtgcccgggctcatcgccctgatcatgaccctggtgggtgccttcctgacctctctggtactggcccgggagtgggagcggggcacgctcgaatcactgttcgtcaccccggtcaggtcgatcgaaatactcctggccaagatcattccctatttcctggtgggcctgctgggcctggtgatgtgcctggtttcggcgcggttgctgttcgaggtaccgatccagggttcgctggtgctcctgctgttgagctccatgctctacctgctggtcaccctgggcattggcctgctgatttcggcgaagacacgcaaccagttcctggccagccagatcgcgatcatcttcagcttcctgccggcgctgatgctctcggggttcctcttcgatctgcgcaatgtgccgacgttcatccgcgtggtgggctcgatcctgccggcgacttacttcatggaactggtcaagaccctgttcctggctggcaacaactggccattgatcgccaagaacctcgcaatcctcgcggcctacgccgtgttcctgttgaacgctgcacgcctgtgcacccgcaagaagctggattgaagccatggacaagctgatgaatttcctacgcgagttgctgttcctgtttcagaaggagttcctggccatcgtcaaggacccggccaaccgggtgatcctgattgccccggcgatcgtccagtcattgctgttcggctacggcgccacctacgacctgaactacgtgccctatgccgtgctggatcagagccaggggcgcgcttcgacagagcttttggcacgcttcgatgcctccggggtatttgcccgagtgtcgaccctgaccagcgtcgatgagatcgccccggtgatcgaccggggcagtgccttgctggtgctgcatatcgccgcagacttcgacgagcgcctgagccggcatgaaaacgccggcttgcagttgatcctggatgggcgtaactcgacgaccgcgggaatggcggccgggcatatcgccaacctggtggctgacttcaaccagcagttcctggcggtggacaacgcgccggtacgcctggagacccgtgcctggttcaaccccaacctgcaaacccgctggaacatagtgccgggcctgatcgccgcgctgagcatgatccagaccctgatgctggcggcgttgtcggtagccagggagcgcgagcagggcaccttcgaccaactgctggtgactccgctgacgcctttcgtgatcctgctgggcaaggccctgccgtcggtactgatcggtctcttgcagtccagcctgatcctcctcatcggcctgttctggttcaagatccccatgatcggctccgtgctggacctgtacctggggctgttcgtcttcaccagcgcctgcgtgggcatcgggctgtcgatctcggcgctgtcggccaacatgcagcaggccatggtgtacaccttcgtgctgatgatgccgctgatcctgctctccggcctgatcaccccggtgcacagcatgcccgaggccctgcaactgctgacctatctcgatccgctgcgctttgccatcgacctggtcaggaggatctacctggagggggcaacgctggcggatgtgtcctggaacctggtgcccatgcttgccgttgcgctggtgaccctgccgctggctgcgtggctgtttcgcaatcgattggtgtgataaggaacgtcatgaaaactactcactctggatacctgcgtaatgggctgacgctggccatgctgctggccatcaccgcctgcaccgtcgggcccgatttcaaggcgccggcagttaccagcccggaacgctggacggattggcacagtggcccgccagcgtcttcggtgccgggcaaagccccggcgaccacctcgcaaaccgcagacggcaactggtggcaggtgtttggcgacccggtgctggaccagctgcaggcccaggtgcgcgaaggcagcccggacctgcacacggccttgctgcggtttgcccaggtgcggttgcaacgccagatagtcgcttccctggaaacgcccgaagtgtccttttccgcggcggcttcgcgaaaccggcaaagccggtatgcgccgaacaatcgaatgcttgaagccttgggcagcgacagtgatgccctggaaaaggtgcttaccgacccttatagcctgtaccaggccgggttcgacgtttcatgggagctggatctctgggggcgggtgtcacgcctgggtgaagccgcccaggccgaggtcgatggtgcggccgcgaccctggacgacgtgttgctcagtgtctccagcgagctggcgcgaaactacttcgaagtgcgtaccgcacagcgccagacccgtcttctggagcaggaggcacagatcctggccgcgcacctgcaagtggttgccgtccaggcccgggagggcgaggaagacgggttcgcggtggaacgccaagaggcacggctggcctcgctgcatgcacagataccgggttggaaggcgctgcacacccaggcgggcaaccgtattgcgctgttgctgggggagcatcccggagccctggaaaccttgctcgcggagcggcctgataatgccttgagccgcccgctgcccaacctgcagttgggcctgcctggcgacctggcccgggagcgtccggacatccgcgcggcccaggcccgcttgcatcgggcgacggcgggcatcggagtggccgaggccgaactgtacccgagcgtgatgctgggggccgacttcggcttcgaatcctacaagagcggccagtttgccgactggagcagccgcacctggtcggtagggccgcgcctggacctgccgctgttcgaccgtgggcggcgcagcaagaccgtggtcctgcgcacactcgaacagcaagaggcggccgtggcatttcaccggaccgtactggcagcctggcaggaagtggacgatgcgatgagccgctaccacaacgaataccagcgtgccgcgcacctcaaggacagctatgacagcaaggcccgcacctatgagtggacccgcgtccggtacgcggccggggaggccagctacctggaggaactggaagcgcagcgcaccgtgctggaggcgcagcgcgacctggtcaatagcgacagtcaactgcgtactcacctgatcagcatttacaaatccatgggtgggcattccacctgagcccctgaaacgctgcctctggcagcactccccccagacccactttcctaacggaataggaagccatgcaaatggatgacctctacaagatcgcgcttttttcgtccctgatactggtagtgccggggccctcaaacaccttgctgctggcctcgggcttcaagttcggcgtgttgcgctcgctgccactggtgttgatcgaaatgctcggctacaccgtttcgatctgcacctggggctgggggcttgtgctgctgtcccacgactacccgtggctgatccacctgatcaagctgctgtgcgccttgtacatcgcggcgctggccttgaagacctggaagacctccaccaccggagcccagcacaccgagcgctttgagagctggccacggcacctgttgattgcgaccttgctcaaccccaaggggctgatcttcgccagtgtgatcttcccggccagctcattcgccagcctgggggatttcctgccgtcattgagcgctttcttcctggcactggcgccaatagccttgctctgggtctcgctgggggcgggcattcgagcccaatacctggggcgggtttctggaccgctgttttcccggggcaccgccgtcgttctggcgctgttttccgccaccctcacttacaccgtggttcgtgcggtgtaggtgtttttgccgcggctttcagcgcccaagtgaggctggggcaatggcgctgctgagagaggcccttgtaccccccgtgttccagcaccactctgttgtcgccaacacccgggggggctcattccacgtagcgcaaccacaaatgggtgcgaccgcgaacagcctcgacaaccgttgaaaccacggcttgccggttttcctcggaggccttgatccgatgaacagggcgtttggcggatgttaatctgcgcctcccggacaaacggtgggccagtggatgcagaggatgttgagtgaagccgagcttcacaaggtgctggaatcgatcgaaagcgactcctgccatgacctgaatcttctgtatgacgggatgcagatagaccggtgtgctcttttcaatcaagtcgccatggcggtggcccggctctttatcgaggggcagcgcgacttccattacggcgatgcggtcatgtgggtcgcggccagnntgggtcgcggccagaaccgccggcgaactcacgcggtttcgctacggctccagcatcaagcggcgccaaagaatcggcggcgcgacagcagcggccggcgcttgcggctccggcttctccggcgcctgggctgacgttgcctcgcggggctcaggcgctcttgtcgactcggtcgggtcatcggtcggcgatggggtcatttgatcctgaccaccaccttgcccttggttcgtccctgctcgacgtactgcaaggcctctgccgtcgattcgaaactgaagctgcggtcaaccacaggcttgatgattccggcctcgatcagcgcggtgatctgttgcagctgggcgccatcggcccgcatgaacacaaaggcgtagcggacgtcccgctggcgcgccttgcgccggataccgaagctcaagaggcgcatgacctgctgcaacggccaggccagcccttgctcctgggcgaattgcaccgtgggtggcccggagatggaaatcagctgtccgccgggcttgaggaccttgagggacttttccagtacgtcgctgcccaggctgttcaacaccaggtcgtagccatgcaagaggctttcgaagttctgctgcttgtagtcgatcacctgatcggcgcccagggctttgacccattcgacattcgccgtgctggtggtggtcgcgacgaaagcgccgaggtgtttggccagctggatggcaagggtgccgacaccgcctgcgcctgcgtggatcagcactttctggccctttttcagttgggcggtttccaccagcacttgccaggcggtgagtgcgaccaaggggatcgacgcggcctcggtcatggtggtgttggcaggtttcagggcgattgcgttttcgttcacggcgatccactgggcgaacgtcccgatccgtgtttcaggcgggcgcgcgtagacttcgtctcccggcttgaagcgttgcacttgggggccaacctcaaccacgacgcccgccaggtcgttgcccagtaccagcggcaatgcatagggcaggatcagcttgaattcgccgttgcggatcttcgagtccagcacgttgacgctgctggcgtggacctcgatcaacacgtcgtgggcacccagcgtgggcgtgggcgcttcgcccatgcgcccactgttcttgccatagcgatcaattagaaaggctttcatcgtggtgcggctcttgtcggttggaataggtgtgggcggtgcttgggtggggttgctgtcatggggctctgtcgtcaaggaacgcccggaccttggccacgaagtccgcgtggtactggaaaatgccgccgtgcccggcgtcctcatacatcactagctgtgcgttggggacacgcttggccagggcatgggagttggcgctgggcaccatgatgtcgttgtcgccattgacgatcagcgtcggcgcccgcaagcgcccgagatcctgcggcgcctgtttgccccacgcggtgatggcttgcagctggcgcaggaaagcgccgggcgttgggcccttgtcgcgatgcttgctgcgggctttcaggcgttgcagatattccgaggcggctcgacgaccatggggcgtcgaggtaaagaacaggtagtacttgggatcgcgcaaggtcagcaagcctttgagcatcaagggccaggtcaccgagccgaccttgtcgataccgatgccgccggccggtccggtgccggtgagaatcaagcggtgcaccaattggggggcttgcagggcgatgtcctgagcgacgaagccgccgagtgagaagcccaggatatcgacgctcttgaaccccagggcctgtatcagctcaatggcgtcttgcgccatgtcgccgaccgtcaggggggcggttccacccgagccgccgatgccgcgatagtcggtggtgatgactcgccgggtctgtgccagcccatcgatgattgccgggtcgaagttgtccagcaccgcgccccagtggttaaacagcaccaggggcacgccgcccgcgggaccggtatcgcgataggcgaagggaatgcccctgaccatgatcgactggttcgccgcgttgatgaacgacgtctgcaaagccggggggaaggttgtctgcgttggattcattgctactccgaacagcggaccggattcgtcgcagtgcctgccgcgaatccgccgcgaatgctcgctacctgaagatcactcttgggtctggtaacgctgggcaacctcggattgacggatgtgccccagcaggccctgacgtgcgctctgcaggtcatcccagggttcagcctgttgcagtggcgggatggtcaccggttcgcgccgatcgaaaccgaccagtgcggcatccaccagatcgcccacttccatgatttcgtccagggtattgatgtcgacgccggagcggtcccagatttccgtgcgggtggcggccggcagcacggcctgcacgtacacgccccggggcgacagttccaggctcaggccctgagacaggaacagcacgaaagccttggttgcaccatagaccgtcatgccgaactccggcgccaggcccaccaccgaaccgatgttgatgatcgcgccgtcaccggctttggccaggcgcggggcaatggcactggccagccgcaccagtgccgtggtgttgagggcaaccagttgcgctacgctgtcggtgctttgctcgatgaagttgccggacagcgcggcgccggcgttattgatgaggatgccgatacgggcgtcgtcgcgcaggcggctttcaacggttgtcagatcgctgagttgggtcaaatccgctttcatcacctcgacggcgaccccgtgttcgccgcgcagtcgggctgcaagtgcgtccaggcgtgcccggtcgcgggcaaccaggaccagatcatggccacgttgcgcgaagcgctcggcgtagatggctccgatgccagtggaggcgccagtgatgagaacggtagggcgagtgttcatggggtcatctctctttcaaaatgggtgaaaacggacagctcggtttctgcttgctcagcttgcgctggcgctcgaatcaaccagcgtcgggtagtcgatgtagcctgccgcgccgccgccgtagagggtggcgggattgaatgcggacaaggcggcgccggtcttgagacgctccaccagatccgggttgccgatgaacgggcgaccgaacgcgatcagatcggcctggtcttcggcgagccgtgaggttgccagctccaggtcatagccgttgttggcgatgtaggtgtttttgaaacgctggcgcagggcggtgaaatccaatggcgccacgtcacgtgggccaccggtcgcgccttcgaccatgtgcaggtaaacgacgtcgagggcgtcgagttgatcgacgacgtaattgaattgcgcctgcggattgctgctggagacaccgttggccggcgacaccggcgacaagcgcaccccggtgcgatccgcgccgatttcattgaccacggcagcggtgacttcgagcaacagacgcgcacgattttcaatcgagccgccgtaagcatcggtgcgtacgttggcgctgtccttgaggaactgatccagcaaatagccgttcgcgccgtgaatttccacgccatcgaacccggcagcgatagcgtttgccgcggcctggcggaaatcggcgacgatccccggcagctcgctgatgtccagtgcgcggggctcgctggcgtcttcaaagcggttgttgacgaacaccttggtggccgcacgcagcgcggaaggggccacgggggcggcgccgttttcttgcagatcaacgtgggacacgcggccgacatgccacagttgcacaaagatctttgcgccctgggcgtggaccgcgtcggtcaccgtgcgccagccatcaatctgcgcctgggtgtagatcccgggggtgtcctgatagccctggccctgttgggaaatctgcgtggcttcgctgatcagcaagcctgcgctagcgcgttggctgtaataggtggcggcgaattcgctgggaacaaagcctgcgcccgcgcggttgcgggtcagtggcgcgaggacgatgcggttcgccagcgcgagggcgccaagggtgtaaggggtgaacagattctgatcggtcatctggtgtctcttccgtgcccgtgggtggaggtcggtggtgtgtgcaattaggatgatgatcgaaatctaaactgtcaacggttttgattatgatcgacatctatgtatgattgggcccatgatttcccaatgaacatgaggtattgcacgtgagagtgagcaaggcccaggcgcaggccaatcgagagcacatcgttgaaacggcctcagagttgttccgtgagcgcggcttcgacggtgtgggtgtgtcggatctcatggcggcggccggtttcacccacggcggcttttacaagcatttcggctcgaaggccgacctcatggccgaggcctcggcctgcagccttgccaagtcgctggcaggcgtgcaggcgctggatgtgcctggcttcatcgacgtctatgtgaccagggaacatcgcgacggacgtggcagcggttgcaccatggccgcgttgtgtggcgatgcggcgcgccaatcggatgatgtgaaagcgacgtttgccgaaggggtcgagcacaccctgcaaaccctgggggacaaatacccgacccggccggatgccgctccggaggagggcagacggaaaatgatcgacctgctgtcccgcgcggtgggtgcgatcatgttgtcgcgtgcctgcccggatgattccgccctggcgaatgagattcttgaggtgtgccgcgctgagatgtttgcttcgttgccggttgataaaggcgagtcggcgtagaggggccccatgccagggccctgcgcttgcgaggtgcctgcttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccacaaatgggtgcgaccgcgaacagcctcgacaaccgttgaaatcactgcttgccggttctcatcagaggtgttgtagagcgcacaaaaatccttcgccacggcgttggcgacgtgcaggtcagcaggcgctgcacgcaggaaaccgaggtaagacgcgcccaccccgatcaactcatagccatgggcctgggcgaaatggcgggccactgcaaagttctgcgcggggcccaggtcgcaactgaagtagccgttgggaaaagcggcgagcgcctgacaggactgctcgacgggcaccaggagtgccgagaggggctggtcgagcagtgatacagggttttgctgatagtccacccattcgtgcagggctggcgcatcaagggtcaggccgtgggcgcaagcgtcttcgaacgagctgcctgccaccaggtcagtgaagttgcgcaagcagatcgcgaactcggccttggtggtggggtagcccaggtattcaccctgttcgaagcattctgcaggtagcagggcgatctcctgctccgaggcatcgtccagccccaggttgttgcgggcgtgcccggaggtgtagcgggcctggggcagatcatccagcgagagaaggggctggaaacccgggtggtcgccagcgatcttgcggtaggcgtcgatcagctcatccaggctggaaatggcgctcaggctgtattcatcgcaggtgccagcggcggggttgccaggcattgcgttaccgcgaaattggatatccatcgaatggctaccggctccatgtgggtgttcaggcgaatgagcaacgctcggtcgaacgtacgtgagcctgctggtgctctcgctgttcgtgccaggcctgtgagttcattccaggcccggggcctagcttaaagagagcacatcctgcctgggcatgaagaagcgtgcaaagagttcggactgtgacttgatgttcaacttggcgtagatgttgcggcggtgcactttgatcgtctctgtggagagggagagctttcctgcgatttccttgttggaaaagccactgagcagcagcctgagcacatcactttcccgcgtggttatctgggtaccaaactgggtgatcgtctccggccattgcggcggttcgctgaggcttttttccacatcgacttcgaagcacatgcgttgatgcatgagtgccgtgacccacggcttgatgatgtccagcatggtgatgtgttcctggctgaaatgggcgtt

SEQ ID NO: 3 - DNA, 41086 bp, Pseudomonas protegens
  misc_feature 1..41086: note = Pyrrolnitrin biosynthesis gene cluster
  source 1..41086: mol_type = genomic DNA
acctgctggcggtgggttatgccggcgccgcgcactaccaggatgtgccccatgcggtagcggaccagcgcgtgatcaccgccccgggcagcgctccagtgagtttcatggcacagattctcggcgccctgggcatcagcgacgacaacctgaccttctacctgggcctgcacgccgccgagcaccgccagcaaacctgaccctcccctgtaggagccggcttgccggcgaaggcgatctcccggccgccttggctggcaagccagcgcctacgcaaggtagtgggtgggcggcagtgcgggcgtgtgccgtggattatttcaccagccgaaaatcccggtggcccaaccaggccgcaaagtccgcagcgctgaaaatgctctcgccctcatgctcgatgatcgccagccccgccagaaattcgctgtcccgggtgtaggcatggatgctggcgccgaaggccagtacgccatccaggtgacgggccaaggtcagcgccgcctgccctaggcgctggtgatccgcctggtctttatccatggccgccaggctcagaatgtcttcagggagccatcccagttgcccggccagcccggcatattccgccagctcccctggatattcggcccccatcccgaacagcatggggctgccgtcaatgaagaacgagctcgtaccggcccagggttgatccgcaattgcggccagacaggcccgcacccggttttcgagcaagccatcgatctggcgcggtaaaagcaccctgacagtcggcccggccattgagtcatctcctccagcgcaccgcatccgcaactgggataacgggcgagcccttgttcgctgcatcagcaggcatcaatgctcaagcccgctgccggtcagtgcattcaccggaccgggagcataaaagcccgtggtgcaatagccaagctcagcgcctggaaccattgacccccaacacctcttccaaccgcttcgcctcgccctccgtcaacaagggcgcaggcggttggacaaggcgctggctggacagggcgggcaccggggccgttggttgggccgtcttcgcagtacgggtgcggctcgcgctgctttgcgggtacagcgcttcatccaggcatttgtcgcgcatgccctggctggacaggccgtggcaaccggtgccgttggacagggcacgggcaccgcaggtggccttgctgtcggggtccttgaggtcgatgcagcccagggaaccaaagccatggcccagtaccgaggcgggatcttcggcgcaagctgcgccggcacacaggaacagggaggtcagcacaagtgcatttcgcataggggcgcgtccatggctagcgtaggtcccccggcgcctttccggccgccgggcaggtctgtcgagggaaaaagccgttgtagcagatgaacatgaccgtttcccttccccataaccaccgctgttgcgtgactagcggaaacgccccgccaggaagggttgcaggttcacgctcggggtcagcccgctggcctgttccgccaccagttgcccactgatggcggccaaggtcaggccaacgtgaccgtggccgaagttgtagtgcacctggggcagcagccgcgactggccgagcaccggtttgctgtccggcagcgagggacgaaagcccatccaccgggaaaccggttcgtggtgcagggtcaggaagcgtcgcgccgcccgggccaggtagtgatagcggcgcgggtccggcgcggcgtcgaggccgccgaactccaccgtgcccgccacccgcagcccttgggccatgggcgtcaggtagaaaccgccggcgccccagcacaccgggcggctgagcaagccgccggcctgatcgaacagcacgtgataaccgcgctcggtgtccaggggaatctcgtcccccaggctctgtgccaggcggcgggaccaggcgccgctgcacaacaccaggcgttcggcggacagttgctcgccactgccgaggcgggcaagcacccgctcgccgtcctgggcgctgcgctcgaccccctgcaactcatcgcggcgaaacactccgccgcgctcgatgaagcaccgcagcaagcgctggctcaatgcgaagggatcgagggtgcggctgacctggggcagttccagcgccagggccgcacgggcgttgagggccggctccatggcccgtagctccggcccttgcaccaggcgcatgggcacctcgaacccctggtgataggccagcatgtcgatagcgctctgccgctcgtgggcctcgaagatgtacagcgagccgtcatggcgcaccaggtcgttggccccggcatcgtcgaacaacgcctgccaggcgcccatgcagggcttgagcaggctgtgcaggtcatgactgatggccttgacccgcggctcgctggagctttgcaaaaagcgccatagccagggggcgatccgtggcaggtaacccgggcggatcgccagcgggctgtcgcgccgcaacagcatgcccggggcctggcgccagatgccgggggttgccactggctccacggcataggtggcgaaggtacaggcattgccgaaggaagtgccgctgcccggcggctggcggtccagcagggtcacccgccagcccaggcgctgcagccacaaggcgctggccaggcccaccaccccggcccccaccaccacggcgtggccgcgttgttgctgacttgcaatcatgccgtgctcctcatgtgccgctgacgacccgggtccacaggcgagtgctcaggcgcatgccggccggggtcatggtcttgatcgggaacagcgtatcgagcaccgactgcggcgggtaggccgccgggttgctgcgcaactgcggttcgatgtagggcaaggacgccgcattgccgttggcgtactgcacgaagttgctgatgttggcgatcaccttggggtccagcaggtagttcaggtaggcgtaggcgttgtccaggtgcggggcgttcctgggaatcaccaccatgtccatggccacggtggagccttccctgggaatgctgtagccgaggttcacgccgtttttcgcctgctgcgcggtggccatggcttgcagcacgtcgccgctgaaacccacggccacgcagatgttgccgttggccaggtcgctgacgtacttggcctggtggaagtagaggatcgaggggcgaatcgcctgcagcgccgcttcggcctttttcaggtccgccggctgttcactgtggggggccaggcccagggcattgagggtgatgggcaccatctgtgtcgggttgtcgagaaaggccacgccgcactgcttgagcttctggatgttctccggcttgaacagcagctgccaggactgggtgacatcggtgtcgccgaagatcgccttgatcttgtcgacgttgtagccgatgcccacggtcacccacatatagggaatgccgtacctattgcccgggtcgctggtttccagcagcttcatcagcttgggatcgaggttgttccagttgggcagcttgctgcgatccaggggctggatcgccccggcctggatcagccgcggcagaaagtggttggacgggctggccaggtcatagccgctgccgccggtcatcagcttggcctcggtggtctcgttgctgtcgatcacgtcgtaggtggtctggattccggtgcgctgggtgaactgggccagggtgtccggagcgatatagccgctccagttggcgatgctcacggtctcggccgcctggacgcttgcggcgcacaaggccagggataacagtgccgtagtggtgcgattgatcatgttgccgccctctgtgctcttattattgcggcgtttcattcgccgcgacggttaacgttcgtgtcgcgcgcgctgcacttcccagcccatgcgcaagcttgctccaatgtcgaaaaacggtcggggtggcaggtagccgggccgggcctgggacagcacgtcacgcagcagcgaggagtcgcaaccggcggccatttccaccagcagccggccccacagggtgccccgcgccacccccgagccgttgcagccggccaccgcgtgcaggccttcggcgacccggctgaagtacggctcgccggaacgggtgccgctcaggtgcccggtccaggtgtattgcaggtcggtatcggtaatgccgggaaagcgcttttgcaggccccgcaggtgttcctggcggcgcaccgccaactgcgccgggccgagatcgcgctggcgatactcgacggtgttgcgcatcagcaggcggcgcccgggcaacaggcgcaccgtggccgccccgggcaaggtggagagcacgccccagccctgctcatccagcagccgctgccagtgatcttcctgcaggggccgggtgatgctggcgctcagttccagggggaaggtgcgactgtgccggacaccgagccggggcaggaaggcaccgacacaggccagcacctggggcgcctggatctcgccctccccggtcaccgcccgccagccccggcccggcagaggctgcaggtcctgcaccgggctgttctcgaacacctccacccgctccggcaggcgctccagcaggcccttgacgtatttggccggctgcagcagcgcgttgccaccggcgcagtggatggcccgctggtagaagccgctgcccaggcgctgctgcaaggccgcgccttccaggtaccgggcctgggcgcccgcggcattcagggtagcgactcgggcgccgctgtcctgcaagtgctgcccctggtgcacggcgaagtgatagccaccagcgtcgaactcgcactcgatgcccaactcgtcaatgcgccggcgcacctcttcaccggcagcgatgccgatgccgcaggcaagctgataggccgagtggccgggccggcccagcaactcatcggcgccgggcaattcatggctgaccacaaaccccgagttgcgcgccgaagccccctgggccgcccgctggcgatccagcaggacgatgcgttgctggggcaacatccgcgccagggcatgggccgccgagaggccggtgatgccaccgccaatgatcagccagtcggctttctgtaggcccttgagggttggccgctgggaagcctgccctgccagggcgatccagccacatacattgttcaaagcctgtcactcctgcacaatccatgcgcccggagcattcaacagaacactcaacccggcgccacatgcctacaatctatggatatgacggctttatttcgaacgttattaattcatccatctattcggaaaacgcatggataaccctcttcgactaccctcgttgcaggccctgcagaccctggtcagcgtggcccagacccacagcttcaccgagtccgcggcgcgcctgcacctgacccagagcgcggtgagccggcagatccagcaactggaggagcactatggggtcagcctgttcgagcgcagcagccgaagggtctcgctgaccgaacaggggcgcgatgtctgcacggtggccactcaagtcctgaggtccctcggctccctgcaggaacgcctggccccggcgcccctggaccggccgtttcgcatccgcatctttgtctccctggcggtgcgctggttgctgccgcggctcagcggtttctacgccgccaaccccgggctgtgcctgtcgatcgagaccgtgggcggggccacggtagaccccggcggcgattgcgacgcctatgtgctgtacctgccggaaggcctggacgagcggccgctgaccccgctgttcgacgaatacctggtaccggtgtgtgccccaacgctcagtgacgggcaattgccgccgcgctccctggaggaactggccgggcacgcgctgatccacggttctaccagccgccaggaatggaccgcctggctgcaggcccaggacgaccagacgccccacagttacaagcacctgctgttcaacctcgacgacctggccctggacgccgcggcccgggggctgggggtggccatgacggacctgatcctggcccaggactcgattgcccggggcagcctgctgatcccctttggccaggcgctcaagaccggcggggtctatgccctgtggctacgcgacagcggcgccgcgcatccggcctgcgaaactgtattgaactggttccaggctcaggcagccgaacgggatctgcccgcggcacaaggcctggctgggacgattgcagcagccctgggcacgcctggacggcagtcgatcccaggctgaatgactggaccaggccctgctgctcatgggcctgggcaaacaacattgaagggccgatgaacactgcccgggaaagctcgagcgccgccggcgctcagctgtagagcgtcaccattgcccaggcaaaacacgccagcaccagggccatccccactacatacaccgctaaagcacgacccacagcatcactccacgaaccaggcagacatcaacacactcactctcggggtgagtcatggcgcgcattctagcagcgcgcgacgtcattttgacacgagactctaagcctgattaaaagcctcccgacaaatggtattggcgtcaatttaatggcttaagtcagcctgattcttcttaccgggtagccgaactgcgaaacatcatcttgcattgcgcccagtgttttcttcccgccaagaaacctgctgcctccaggatcaatgcaagcccccttttcccgctaaaccgcgaacctgagcgctatcagcgcctgcactcctggttggacaagagtgatcctgactgctttgatctcaacgccaggtccggttttcacaatcccagggaggggtccatgtcgttcaaggtcatgatggttttcggtacacgccctgaggcgatcaagatggcaccactggctcgggttctaaggcactggccaggtatcgagctgcacatctgttccaccggccagcatcgggaaatgctcaaacaggtactcgacgcctttgagctcgaggtcgatgtggacctgcaagtgatgacccagggccagaccctcaacggcttgtcgcagcaactgctgatccatctggacaccagctacgagcgtctgcaaccggacatcgtgctggtccatggcgacaccaccaccagttttatcgccgccctcgccgccttcaaccggcagttgcccatcggccacgtcgaggccggcctgcgcactggcaacctccaggcaccctggcccgaagaagccaaccggcgcctgaccggggtcatcgccgacctgcacttcaccccgaccgccaggtccgcaaccaacctgctgcgggaaaacgtgcccgctgaaaacatcgaagtcaccggcaacacggtgatcgacgcgctactgtggatgcgcaaccggcagaagcacacccactggcgccccgcggccaactcgcccctggcggtgctcgacgacagccggcgcatggtgctgatcaccggccaccgccgggagaacttcggtgacggtttccgcgatatctgcgaagccctggcaaccctggccgagcgctacccggacgtgcagttcgtgtacccggtacacctcaacccccaggtacagaacgcggtgtacggcctgctctcgaacaagccgaacatctacctggtagcaccccaggactatcagcattttgtctggctgatgggacgttcgcacttcatcctcagcgactccggcggtgtccaggaagaggccccggccatcggcaagcccttgctggtgctgcgcgaggtcaccgaacggccgtcggtgctggaaggcggcaccgtgctgctggtgggcaccgatcgcgagcgcatcgtgcaccacgccagcgaactgctggacgatgcacagctgcatgcccgcatgagccgcgtgcacagcccctacggtgacggccgcgccagcgaacacatcgcccgccgcctgtgctcctggctggaggcccaagccccccgtactccggcatgagcccgggcagccccccgcagcccgcagtcgcaaaacgtccagttggccatatggctccacactctgtgcgagaactggaagctaccggtcactggcaggtacctgccaattgaactggacgacttacactttcaagcagcggatcgtcgcgccccctccaaccccagcgcggtgttctccctgcgctatcgcccctggggcaagccgctgcaggatcatcagctcacatccaaggcccgcgatgttcagaacccttgaagaacaagtgttcaaacaggccgctccggcggccctgaagcttgaattcgcgcaacatgatttcgaacgcctgcgcagtttctgcgtcctgatctacaccgccagcatcctgatctggctgatcttcgacctgatcgtcagctttcccgggcagcagggcttcaccgcctattcgatggtctttctgagcattctgtccttcaccaccatcatcctcggcttcacccgccagggccggcacttccagtgggtcaacctgctgttcgtgctgaccatcaccatcggcgtacgcctggtgatcgaggggctgcccatcgaccagcgcccggtctggctgattctcggcgcgtcgagcatgctctacggcgcctcggtgatgcctctgcgacgctggtcgttcgtcagcgcgatcttcatcacttggctggtgctcaatccgttctggcgcaccagcaccaccctgcaggaactcaaggggctgctggtgctggtgtacgcgctgtttctcagcgcattgaccctctacagcttcatcaccctgcgccgggcccggctgcacaactacatcatgtccaagttgctgctggaccaggcctacctcgacaccctcaccgagatccccaaccgccgctcgttcatggcccgtgccggccagcgcctgcaggctctgccccgggagcacgaccattacctggcgatggtcgacatcgacaacttcaagaaggtcaacgatgtgtacgggcatgacatcggcgacgaagtgctcaagcgcatcgccgccgatatcaagggagtgatgagggatttcgactatgcccgcctgggcggcgaggagttcgccatctacctggccgacgtgccccgcgcggatgtggaggcgctggccacgcgactgtgccaggtggtgcgcgaccagccgacccggcacccggtgaccatcagcatgggcctggcccgggtcgagcctggcgaaaccctgaaccaggccctgatcaaggccgaccaggcgctgtacgaatccaagcacggcggcaaggatcgctataccttccatccagccgccaggcccgcaccacccacgtaggagctggcttgccagcgaagaggccccagtgtcagtccacgctcctgcgcgacagcagcacctgggttacccggcgctcctccactgccctgacctgcaggctccagcccgcccactccacccgatcgcccttgaccggcaggcggtccagcaggctcatcaccagaccggccagggtctggtagtcctcggtgggttcagcgccgaagccggtccgctggcgtacctgaggcaggttcagcgcaccattgaccaggaacccgccctcttcttcgatcacatccgggccttcgatctcgctggcatccggcagctcgccggcaatggattcgagaatgtcggtcatggtcaggatgcccatgaagtcaccgaattcgttgatcacaaaggcaatgtgagtggacgcctggcgcatctgttccagggcgttgaggatcgaaaagctgtccagcaggttgatggtcttgcgcgccagatgctcaaggttcggctcactgcccgccaggtactccttgagcagttccttcttgtgcacgaagcccaaaggctcatccaccgcaccatcgcgaatcagcggcaggcgcgagtacgaagaatgcatcagcttggtacgaatactctccgggctgtccgtaaggtcgatgcagtcgacctcggcacgcaccgtcatcaggctgcggatcggccgctccgccagttgcagcacaccactgatcatcacccgctcacggcggtcgaacagctcgctgctgggggcctcatcagtttccagcaagtcggcaatttcctcgcccacttcttccgccgccagcctgcgcccacccagcaggcgcatcaccgcataggccgtgcgctcgcgcatcggccgcaggccctgcatggatttcttgcgccgggcccgggcgatctggttgaacacctcgatcaggatcgagaagccgatggccgcatacaggtaacccttgggaatatggaaacccaagccttcggcggtcaggctgaagccgatcatcatcaagaagcccaggcacagcatgatcaccgtcgggtgcgcgttgacaaagcgggtcagcggcttgctggcgacgatcatcacgccgatggacaccaccaccgcgatcatcatcaccgacaggtgctcgaccatgcccaccgcggtgatcaccgcgtccagggagaacaccgcatcgagcaccacgatctgcgccacgatcggccagaacaaggcgtaagtggtactgccggcgcgctggctgacatggccctccaggcgttcatgcaactccatggtggccttgaacagcaggaacacaccaccgaacagcatgatcaggtcacgccccgagaagctgtgctcgaagacctcgaacagcggctgggtcagggtcaccatccacgagatgctggccagtaggcccaggcgcatcaacaacgccagggacaggccgatgatccgcgcacggtcgcgctgctcgggcggcagcttgtccgcgaggatggcgataaacaccaggttgtcgatgcccagcaccagctccaggacgatcagggtcaaaaggccaagccaggccgtggggtctgcgatccattccataacgacgactcaagtgctccggaaaattcaaacggccaggcgcgacgcagcgccaaggtgcaggggctgcaaaacctcgggtacgggcggataaaggcaggaagtggcaagtgccaggcaagtgcaaccgggagaatggctgcctgagcgtctggggctcagggccgccggatggcgcgagaagtcatggggcaaagcgaggaaagagcgccgactggggggctccgagagcgtgttcatgcaagtcctgaaggaaaaaacggacttggagattacagggagggcaaggcgattccccagcgggaaatcattacaagattttatccggcccgcgtctctgacgcaggccggcaccgctacaactgctcggaaatctcgatcaggttcaggtccgggtcccgcaggtacaccgaacggatcgggccggtggcgccggtgcgcggcaccggcccttcgatgatcggccagttggcctgttgcagatgggcaatcacctgttccaggggcacgctggcgatgaagcacaaatccagcgcccccggcaccggcaggtgggccttgggttcgaactcctggccgcgcacatgcaggttgatcttctggttgccgtacttgaaggccttgcgcccgccggcaaaggtttccagttgcatgcccagcacgcggacgtagaaatcctcggcggcaatcgggtcgatggtggtcagtaccaggtggtcaaggtggtcgatcatcgaagacgcggctcctgaagcgaaaggaatggcccggggattatgcgacccgggccgtcgcttgaacatgccccatcaggctttgccgcggtactcgcgccgtacctgctcgatgtagcgcaccaccgccggtgcctgctcgaaacgccggtggatcagcgacagccaggacgtcgccgagctgccgccaatcggccggtacagcacatggggcaggctgatgtggcccaccaccgactccggcaccaccgccacgccctgccccagggacaccagggccagtaccgccaccaggccgccgggctggggccccagcttgggggcgtaggcaccctgggccgccacctgcaaggtgccgctgatctgctccggaagaatgaaggtttcattgagcaggtgttcggaggcgatctgcggcaggcggcacagccaggattcgtcggacagcgccaggacaaagccctcggcgtccaggcgaatggcctcgaccccttccggcaatgtcatgggcgagcgaatgtagcccacgtccaggcgaccatcggcaatccgcgccggcagcgtcgccatcgggcactcccgcacgttgaggctgacgtccgggcagtcgcggccaaagccctgcacctgtttctgcaacagcccgcaatacaccgccgaggccacatagcccagttcgatatggccgatctcgccgcgcccggcacgctgggcattgcgctgggcaaactcgaactggcgcaccgtggcctcggcttccagcagcagggtggcgccggcctcggtcaggctgacctcgcgttgctggcgcaggaacagccgggtgccaagggtgttctccatgtcctggatctgccggctcagggtgggcggggcaatccccaattgctcggcggcgcgggtgaaattgcgctgccgggccaccgccaggaagtagcgaaagtgacgaatttccatgatgggcattagctcaaaggtaatgaactggtcgccagcagctaacaaagaccgcgaaaggccgggataagctgagcgaaccttcacagtagagagcctctgccatgcgcgtcaaacccctgtttctggcaatgatcacctgctgcgtgctgcagaccgcggccgccgaggacagccaacccgtcattgcccacaaggcaatccttaccctggacaacgcccagcgcctgctcagggccgcccagcaaaccgcccaggccaagggctggccctgcgccgtggcaatcgtcgacgacggcggctggccgatactgggcgcgcgcatggacggcgccccagtggtggccggagtcgagctggcccagggcaaggcacgcacctcggcattgttcaagcggccctccggggatctggaaaacgccgtcaatggcggccgccccgccgccctcagcgccggcctggtgatgatgaaaggcgcccagccgatcatcgtcgacggccaggtgatcggcgccatcggggtcagtgccgacaccccggcccacgacgatgaaatcgccctggccgccctcgccaccctcagtcggcaggccacgcaatgaaccgctccgtcaacccgcgcctgaccctgctcaccgcatccggggtctgttcgctgatcgttctggacaccaacatcgtcgccgtggcgctgccgagcatcgcccgtgacctgggggccaacttcgccgatatcgagtgggtggtcagcgcctacatgctggccttcgccgccctgctgctgcccgccggcagcctggccgaccgcttcggccgcaagcgcaccctgctctgcggcctgggcctgttcatcctcgcctccctgggctgcggcgcggcgcccaacgccctgctgctggacatcgcccgggcgctcaagggggtgggcgcggcgctgctgctgacctcggccctggcctccatcggccataccttccacgacgaagtggaacgggccaaggcctgggccttctggggcgcctgcatgggcatggccatgactgccgcgcccacggtcggcgggctgatcaccgagtacttcggctggcgctggatcttctacttcaacctgccggtgggtgcgttgctgctgtggagtgtgctacgcaacgtgccggaatcccgggatacccaggcggcgcgcctcgacccctggggcagcctggccttcagcgccagcctgctgtgcctgatctggggcctgatcgaggccaaccgcatcggctggagccacccggcgaccaccacccgcctgctggcaggcgtggcgctgctggggctgttcgtgctgatcgagcggctgcaaagccggcccatggtggacctgcaactgttccgccacccgcgcttcatcggcgccctgctgggcatgttcgcctacgccggctgtgcccaggtgatgatgacgctgctgcccttctacctgcagaacggcctgggcttctcggccatcgcctcgggcctgggcatgctgcccttcgccctgaccatgctcatctgcccgcggatcggcgcacgcctggccgggcgctatgcgccggccacgctgatggccgcagggctgaccctggtgggctgcggcaacctgctcagcgcctgggcggtacacgccgggggctacctgaacttcgccctggcaatcgccgtgaccggcgccggggccggattgctcaatggcgacacgcagaaaaacatcatggcctgcgtgccgcgggaacgcaccggcatggcctcgggcatgagcaccaccatgcgctttagcgccatcatgctggccatcggggtgttcggtgcgctgctggccagccatacccagcagctcttgcagggcagcctgcacgccgtgcccgggcactggctggagcaggcaccggagattgcctcacgggtggtggccggggatatgcagggcgcgatggccggcttgccgcccgatgcccgggccctggtcgagcccctggcccggcaggcctttgtcggtggtttcggcctggtgctgacggtggccgggatgctcgccctgctcggcgccctggtggtgggccggctgatgcgcaatccactgccccaggcgtcggggctgacgctgaagctgcaatagcccaggcactgcacaaaccgctgccgcgcctgcggcagcggtcctggcccgcccgcttgcaatttcacaacactcgttcaattgctggggcaatccgccacggttaaggtgcgcccttcgcaacgccccattgaatggataacgaggtgagcgtgaagccttcaagcgtgatcgtggtgggcgccgggattgtcggcgccgccattgcctaccacctggcccggcagggcgcgcaggtgaccgtgctggaacaggacagcccgggctcgggcgtcacccgccactcctttgcctggatcaacccccacggcgagttcccggcagccgcccgcgccctgcgggcccaggcgctggcagactaccgccgcctgcagcacgaactgccggccttgcagatccgctggtccggcgcgctgacctacggcgcggccgaccagcaagcgccagataccgacgagccaaccgccgaggcctcgacccggtggctggaccgggccggcgttgcccaactggaaccccacctgcaagtgctgccggagcgtgcccgctatgcacccggcgaaggcagcctggacccggtggccgccacccgcaccctgctcgatgcggcctgcacccacggcgcccgggtgttcgaccagacccgggtacttagcttgcgggtcgaaggccagcgggtactcggggtaaacaccgccgccgggctccaccgtgccgaccaggtgctgctcgccagcggctgcgccaccccggccctgctcgcgcccctgggtattcagttgccggtgcaggcctcgccgtcgatcctgatccgcgtcaatgcgccaccgggcctggtcaagaccctggtctccaacagccagctggaaatccgcgaagcggcggacggcagcctgctgctggccgaggactatatcgatgcccagggtccactggggcccgaggccatcgcgcaacaggccctggccagcatccgccagctgttccgtggcgcccaggccatcaccttgcgcagcgtggaagttggcctgcggccgatgcccggggaccaactgccgatcatcggccccagcacccgctaccagggcctgtacctggcggtgatgcacgccggagtgaccctggcccccacggtggcgcgcctgatcagccaggaactgctgcacgggcaaacctgcgccgaactggccgcctgccgaccggcacgcttcgcctgaacaccagcccctgtcgccacaccgcggcagggcctgcaagatttcacggaatcatccgccctcgcctgttgtcctcttttgattgcacgacttcttaagcggaccgcaggagctcttcatggcgaacatcaccatcgcccagcaactggcaaccacccttgaacaggccggggtcaagcgtatctgggggctgaccggcgacagcctcaacggcctcaccgacgctctgcacagcatgcagaccatcgagtggatgcatgtgcgccacgaagaggtggccgcctttgccgccggcgccgaagccgccaccaccgggcaactggcggtgtgcgccggcagctgcgggccgggcaacctgcacctgatcaatggcctgttcgactgtcaccgcaaccatgtgccggtactggccatcgccgcacagatcccctcctcggagatcggcctcgactattttcaggaaacccaccctcaggagctgttcaaggagtgcagccacttcgtcgaactggtgagcaacccggcgcagatgcccgaggtgctgcaccgggccatgcgcagcgcgatcctcaaccgcggcgtggcggtggtggtgatccccggcgacgtggccctgcagcaggtacccgccgccagcaagccatggccggccctgcacgcaccgcgcaccctgcccgccccccacgacctggacaaactggtggagttgctgagccagagcaaggccgtgaccctgctctgcggcagcggctgcgctggcgcccacgatcaggtggtggccctggccgacgccctcggcgcaccggtggtgcatgccttgcgcggcaaagagcatgtggaatgggacaaccccttcgacgtcggcatgaccggcctgatcggcttcagctccggctaccacgccatgctcaactgcgacaccctggtgatgctcggcaccgactttccctatcgccagttctaccccagcgacgcctgcgtcatacagatcgaccgcaacccccaggccctgggccggcgagtgccgctggacctggggatcgccgccgacgtcggcgaaaccctcgccgcactgctgccgcgcctgccctataagggcgaccgccgctttctcgaagaatccctcaagcactacgccaaggcgcgccagggcctggacgacctggcccagccctcgcccgctggccggccgatccacccgcagtacctgacccggctgctcagcgaactggcagacgaagacgccatcttcactgccgacgtcggcacccccacggtgtgggccgcgcgctacctgaaaatgaacggccagcggcgcctgctgggctctttcaaccacggctccatggccaacgccatgccccaggccatgggcgcccaggccgccttcccccagcgccaggtgatctcgctgtccggcgatggcggcttcagcatgctgatgggcgacttcatctccctggcccagctcaagctgccggtgaagatcgtggtctacgacaacgcctccctgggcttcgtggccatggaaatgaagtccgccggctacctggacaccggcaccgacctgcacaacccggactttgccgccatggccaacgccatgggcattctcggcctgcgggtggaaaactccgaagaactggaaggcgccctgcgcaccgccctggcccatgacggcccggtgctggtggacgtggtcaccgccacccaggaactggccatgccgccggccatcaagctggaacaggccaagggcttcagcctgtacatgctcaaggcggtgatgagcgggcgtggcgacgaggtgatcgaactggccaggaccaattggctcaaggacagttggttcaagatcacctagaaaggctctacccccgggcatgccatcgcagcctctaatgcacccacattagaggcctgtgtccatgaaaagggtggcagtagcatccatcgagggtggcgttgggaaaaccaccacctccgcaggcctcgcctcttatccattaagacaagacgtttcgactgcacgccaacggattggccgtaagcacaagcgggacaaggaagccgtcaggctctgtcagtgatccgatcatttttgagcggctcgcctctttcaagatcggcagcggatgaaggcatgcccaaacgtcagcccccccctcagcggccgactgatcaggcgcaaaaaacctacgcaatattttcctttagaccaaatgcctttgcttttttgaatgctaattcggaaacggaaggtgcatcgcttttccggcaggctagagtctaaatccgacaagcacattgatgtgcctcttgcacggatgcacagagactggcggccttcccctcctcacagacagcccgcctcgaaacagggagtgttatgaacaagccaatcaagaatatcgtcatcgtgggcggcggcaccgcgggctggatggccgcttcgtacctcgtccgggcgctccaacagcaggtaaacatcacgctcatcgagtctgcggcgatcccccggatcggcgtgggcgaggcgaccatcccgagtttgcagaaggtgttcttcgacttcctcgggataccggagcgggagtggatgccccaagtgaacggcgccttcaaggccgcgatcaagttcgtgaactggagaaaatctcccgacccatcgcgcgaagattacttctaccatttgttcggcagcgtgccgaactgcgacggcgtgccgcttacccactactggctgcgcaagcgcgaacagggcttccagcagccgatggcgtacgcgtgctatccgcagcccggggccctcgacggcaagctggcaccctgcctggccgacggcacccgccagatgtcccacgcgtggcacttcgacgcgcacctggtggccgacttcttgaagcgctgggccgtcgagcgcggggtgaatcgcgtggtcgacgaggtcgtggaggttcaactgaacgaccgcggctacatctccaccctgttaaccaaggaagggcggacgctggaggcggacctgttcatcgactgctccggcatgcgagggctcctgatcaatcaggccctgaaggaacccttcatcgacatgtccgactacctgctgtgcgacagcgcggtcgccagcgccgtgcccaacgacgacgcgcgcgagggggtcgagccttacacctccgcgattgccatgaactcgggatggacctggaagattccgatgctgggccggttcggcagcggctacgtcttctcgagcaagttcacctcgcgcgaccaggccaccgccgacttcctcaaactctggggcctctcggacaatcagcagctcaaccagatcaagttccgggtcgggcgcaacaagcgggcgtgggtcaacaactgcgtctcgatcgggctgtcgtcgtgctttctggagcccctggaatcgacgggaatctacttcatctacgcggcgctttaccaactcgtgaagcacttccccgacacctcgttcgacccgcggttgcgcgacgcattcaacgccgagatcgtctacatgttcgacgactgccgagacttcgtccaggcgcactatttcactacgtcgcgcgaagacacgccgttctggctcgcgaaccggcacgaactgcggctctcggacgccatccaggagaaggttgagcgctacaaggccgggctgccactgaccaccacctcgttcgacgattccacgtactacgagaccttcgactacgaattcaagaacttctggttgaacggcaactactactgcatctttgccggcctgggcatgctgcccgaccggtcgctgccgctcctgcagcaccgaccggagtcgatccagaaggccgaagcgatgttcgccagcatccggcgcgaagccgagcgcctgcgcacgagcctgccgacgaactacgactacctgcggtcactgcgtgacggcgcgcagctgtcgcgcaaccagcacgggccgacgctcgcggctcaggaacgccagtagtggaacgcaccttgaaccgggtatccgcattcgcggccacacacgctgccgtggcggcctgcgatccgctacaggcacgcgcgctggttctgcagctgccggccctgaaccgtgacaaggacgtgcccggcatcgtcggcctgctgcgcgatttcctcccggtgagcggcgtgccctccagctggggcttcgtcgaagccgccgccgcgatgcgggacatcggtttcttcctggggtcgctcaagcggcacggacatgagcccgtggacctggtgcccgggcttgaacgggtgctgctcgacctggcacgggtgaccgacttgccgccgcgcgagacactcctgcatgtgacggtctggaacccggcggcggccgatgcgcagcggagctacaccgggctgcccgacgaagcgcacctgctcgagagcgtgcgcatctcgatggcggccctcgaggcggccatcgcgttgaccgtcgagctgtccgatgtatccctgcgctcgcccgcgttcgcgcaagggtgcgatgagctggaagcctacctgcagaaaatggtcgaatcgatcgtctacgcgtaccgcttcatctcgccccaggtcttctacgatgagctgcgccccttctacgaaccgattcgagtcgggggccagagctacctcggccccggcgccgtagaaatgcccctcttcgtgctggagcacgtcctgtggggctcgcaatcggacgacccagcttatcgagaattcaaagagacatacctgccctacgtgcttcccgcgtacagggcggtctacgctcggttcgccacaaagccggcgctcatcgaccgtgcgctcgacgaggcgcgagcggtgggtacgcagggcgagcacgtccgggctgggctgacggccctcgagcgggtcttcaaggtcctgctgcgcttccgggcgcctcacctcaaattggcggagcgggcatacgaagccgggcgcagcggccccacaaccggcagcgggggctacgcgcccagcatgctcggcgatctactcacgctcacctgtgccgcgcggtcccgcatccgtgccgcgctcgatgaatcctgatgcgcgcgacccagtgttatctcacaaggagagtttgcccccatgactcagaagagccccgcgaacggacacgatagcaaccacttcgacgtaatcatcctcggttcgggcatgtccggtacccagatgggggccatcctggccaaacaacagtttcgcgtgctgatcatcgagcagtcgtcgcacccgcggttcacgatcggcgaatcgtcgatccccgaaacgtctctcatgaaccgcatcatcgctgatcgctacgacattccggagctcggccacatcacctcgttctactcgacgcagcgttacgtttcgtcgagcacgggcatcaagcgcaacttcggcttcgtgttccacaaacctggccaggagcacgacccgaaggagttcacccagtgcgtcattcccgagctgccgtgggggccggagagccattattaccggcaggacgtcgacgcctatctgttgcaagcggccatcaaatatggctgcacggtccgccagaagacgagcgtgaccgaatatcacgcggacaaggacggcgtcgcggtgaccaccgccgagggcgagcggttcaccggccggtacatgatcgactgcggaggacccggcgcgccgctggcgaccaagttcgggctccgcgaagagccgtgtcgcttcaagacgcactcgcgcagcctctacacgcacatgctcggggtcaagccgttcgacgacatcttcaaggtcaaggggcagcgctggcgctggcacgaaggaaccctgcaccacatgttcaccggcggctggctctgggtgattccgttcaacaaccacccgcgctcgaccaataacctggtgagcgtcggcctgcagctcgacccgcgtgtctacccgaaaaccgacattcccgcgcagcaggaattcgacgagttcctcgcgcggttcccgagcatcggcgctcagttccgggacgccgtgccagtgcgcgactgggtcaagaccgaccgcctgcagttctcgtcgaacgcctgcgtcggcgaccgctactgcctgatgctgcacgcgaacgggttcatcgacccgctcttctcccgggggctcgagaacaccgcggtgaccatccacgcgctcgcggcgcgcctcatcaaggcgctacgcgacgacgacttctcccccgagcgcttcgagtacatcgagcgcctgcagcaaaagcttttggaccacaacgacgacttcgtcagctgctgctacacggcgttctcggacttccgcctatgggacgcgttccaccggctgtgggcggtcggcactatcctcgggcagttccggctggtgcaagcccacgcgaggtttcgcgcgtcgcgcgacgagggcgacctcgatcacctcgacaacgacccgccgtacctcgggtacctgtgcgcggacatggagcagtactaccagttgttcaacgacgccaaagccgaggtcgaggctgtgagcgccgggcacaagtcggccgaggaggccgcgttgcggattcacgccctcatcgacgaacgagacttcgccaagccgatgttcggcttcgggtactgcatcaccggggacaagccgcagctcaacaactcgaagtacagcctgataccggcgatgaagctgatgtactggacgcaaacccgcgcgccggcagaggtgaagaagtacttcgactacaacccgatgttcgcgctgctcaaggcgtacatcaccacccgcatcggcttggctctgaagaagtagtcggccaaggacggcacacacgcgatgaacaacattcaattggatcaagcgaacgtcaagaagcatcccccgggggcgtacgacgcgaccacacgcgtggccgcgagctggtacgtcgcgatgcgctcgaacggcctcagggacaagccgaaggagttgacgctctttggccgtccgtacgtggcgtggcgcgcagcgacggggcaggccgtggtgatggaccgccactgctcgcacctgggcgcgaacctggctgacgggcggatcaaggacgggtgcatccagtgcccgtttcaccactggcgctacgacgagcaaggcaagtgcgttcacatccccggccacagcgaggtggtgcgccagctggagccggtgccacgcgcggcgcgccagccgacgttggtcaccaccgagcgatacggctacgtgtgggtctggtacggctccccgcagccgctgcacccgctgcccgaaatcaccgcagccgacgtcgacaacggcgacttcatgcacctgcacttcgcgttcgagacgacgacggcggtcttgcggatcgtcgagaacttctacgacgcacagcacgcaacccccgtgcacgcgctcccgatctcggccttcgaactcaagctcttcgacgactggagccggtggccggaggttgagtcgctggcccgggcgggcgcgtggttcggtgccgggatcgacttccacgtgaaccgctacttcggccccctcggcatgctgtcgcgcgcgctcggcctgaacatgtcgcagatgaacctgcacttcgatggctaccccggcgggtgcgtcatgaccgttgccctggacgcagacgtcaaatacaaactgctccagtgtgtgacaccggtgagcgacggcaagaacatcatgcacatgctcatctcgatcaagaaggtgggcggcgtcctgcgccgtgcgaccgacttcgtgctgttcgggctgcagaccagacaggcagcggggtacgacgtcaaaatctggaacgggatgaagcccgacggcggcggcgcttacagcaagtacgacaagctcgtgctcaagtaccgtgcgttctaccgcggctgggtcgaccgtgtcgcgagtgagcagtaatgcgtgaggccgagccggtagcggtcgcgtcgcgctgcccggcgcttgcgaacctttcgagctgcgtcacggagatcacggcgtacggcgcggcgggcccgcttgggctcgcggccacccgcttggtgtcggtgtcgctctttgcgaggtattgatgaccatctggctgttgcaactcgtgctggtgatcgcgctctgcaacctctgcggccgccttgccgaacggctcggccagtgcgcggtggtcggcgagatcgcggccggcctgctgttggggccttcgctgttcggcgtgatcgcaccgagtttctacgacctgttgttcggcccacgtacgctgtcagccatggcgcaagtcggcgaagtcggcctgatactgctgatgtttcagatcggtctgcatatggagttgagcgagacgctgcgcggcaagcgctggcgcatacctgtcgcgatcgcggcgggcgggctcatagcgccggccgcgatcggcatgatcgtcgccatcgtctcgaaagatacgctcgccagcgacgtgccggcgctgccctacatactcttttgcggtgtcgcacttgcggtatcagcggtgccggtgatggcgcgcatcatcgacgacctggcgctcggcgccatggtcggcgcacggcacgcaatgtctgccgcgatgctgacggatgcgctcggctggatgctgctggcaacgattgcctcgctatcgagcgggcctggctgggcatttgcgcacatgctcgtcagcctgctcgtgtacctggtgctatgcgcgctcctggtgcgcttcgtggtgcgaccggtgcttgcgcggctcgcctcgactgcgcatgcgacgcgcgaccgcttggccgtgttgctctgctttgtaatagcctcggcactcgcgacgtcgctgatcggattccatagtgcatttggcgcacttgcggcggcgctgttcgtgcggcgcgtgcccggcgtcgcgaaggagtggcgcgataacgtcgaaggtttcgtcaagcttgtactgatgccggtgttcttcgcgtgtgcggggctgcatgcgtcggtcggcacgatagacgacgcggcatcatggatgtggtgcggggtattcctcgtgggcggattcatcggcaagttcggcggcagctatctgggcgcgcgtggcactgggctggcgccacacgatgcgatgctggcgagctcgttgatgaatacgcgcgggttgatggagctaatcgtcctgtcaatcggcctgcagatgcagattcttccgccaaaggtctacacgatcctcgtggtgttcgcgctggtgacgacggcgctgaccgcaccgctggttcgattcacgctgcgcatgcaaacacgcgcaatgagccaacaggccgcctgagccagcttgaggggagagctcaccatgaatgctgccaccgaaaccaaagttcacgatttgctcgatgccgagggccgcgatgtccgcgatgcccgtgaactgcgcaacgtgctggggcagtttgctaccggtgtgaccgtaatcaccacccgcaccgcagacggccgcaacgtcggtgtgacggccaactcgttctcctcactgtcgctgtcaccggcgctggtgctctggagcctggcacgcacggcaccgagcctgaaggtcttttgctcggcgagccacttcgccatcaacgtactgggcgcgcaccagctccacctgtcggagcagttcgcacgggcagcagccgacaagttcgccggtgtagctcattcctatggcaaggcgggagccccggtgctggacgatgtggtggcagtgctggtgtgccgcaacgtcacccagtacgagggcggtgaccacctgatcttcattggcgagatcgaacaataccgctacagcggtgcagaaccgctggtcttccatgcaggccagtaccgggggctagggagcaatagagcagaaagcgtcctcaagcacgaatagcccggctcatgtgatcttttgccgggaaggatcactgatccaggtgccccttgatgtggcagcagctcaacctcagtggctcgctgcgcagcgccccagcgaacaaccagcccgaccagccaagcgcccgcgacagctatttgtagtttccctcctcctcctcctcnncctcctcctcctcctcctcctcctcctcctcctcctcctctgaagtcgatccgacacccacgccaggaaatgaataatcactattgggtttgtgcccaatgagaaagcccttgtagtacgtttttttcgaatgccttaggcccttgaagcctggccatcccagcgcgaggcttgcccaggcgctgctgattgaggctcacaaaatgcatttgataatcatttgcaatcaaggatagaatcccgccccctcgtctgatcccgccttgaatgatcgccgccgtgtcttatctcagcgataaactcgagctctacctttcccaccgtaccgcgctggtcgactattcggcgcccattgtcggttgtcgggctcaagccgaagaagtggtccaggaggcctggctgcgcttctgcggccaggccgatagcgccgcccagccgagcaacccggtgggctacctgtaccgcatcgtgcgcaacctgtcctttgacctgctgcggcgctcgaccctggaaaatcgtcatcccgatggcgccgatctgctgaacgagctgccctcgagtacgccatcaccggagcatcaggcgctgcacagcgaccagttgcgcctgctgcaagatgcgctggcactgctgccggagcgcacgcgccgggccttccacatgcaccgcctgcaacacctgaccttccaggaaattgccgctgagctgaagatctcctccagcttggcgcaccagctggtgcgtgacgcactgacccactgtgcggagcacctggccgatgactgatacccccttgcctcaacctgtatccggttcactgaaggacgaccccgtgtggcaggcggcgatggattggctgctgcaatgccactcggcgccggatgacgccttgctgcaacaggcccatgcgcgctggctggccgctgacgagcgccatgctgtcgcctggcgcaaggccgagaaagtctggttgctcagtggcggcctggcgccgctggaaccacccgtgccccagcctttgcccaccccgctccgggctcggcgcaacaggccgcggcgggcactgaaggcgcttgcactggcagcctgcctgctgttgctggccgggccgacaccaccgaccgcccataccagcccagccggcgagcaccgacaagtgctcttgagtgacggcagccgtattgaactgggcagcgacagcgctattcgagtggacttcgaacccggtacgcgggcggtcaccctgctcagggggcaggcgttcttcgaggtcagccacgatgcctcgcgcccgttcacggtgcaggctgcggacgtcaaggtcagggtcatagggaccgcgttcgacgtggacctcagccgcaccgccgtggtggtagccgtacagagcggcgctgtccaggttcgcgatgggcgcggcgaactcgcggtgccggccctgggcccgggcgatagccttcgccttggcctggaccagggcccgccgcagcgcggtcggctattgcctggccaggttgccccctggcgccagtggcagttgctggtcaatgaccggccactctccgaggtggttgaagcgctgcaggactattaccccggtgtactgctattgacggaccctgctctgggcgagcggcgcatcaccgcttcgctgaacctgcgctcgccagtcagcgcactgcaactggcgatcgctccgttgggtgggcatctgcggcaatggggcccctacctcacgctgatccgcaaagagccgcaagtgccggcaaaacaataaatttcaaaattttctggaagttctcgatcgccgttcgtttattgccccgctgagttaatcataattattctcattagcatgtgcgtattgttcgcactgaacggggagttcaccagagcatgtccattttcccccaacagggacttcagggttgccgacgcagtgcgcggctgttgtggctgtcagtggggctggcggtgtccctgccggccttggccagtgaccatcggacacggcatagcatcgacatcgcacgccagccgttaagcacggcgctgatggcgctggcacagcagaccggcctgcagatcagcgtcgagagccaactgctgctcaacctgaacgctccggcggtgcagggcgaactgagcgccgagcaggcgctggcgaagctgctcaaaggcagccagctggagtggatctacatcggtgacgacgcgctgatggtgcaacgggcgactcgcccgcgtaccgacccggtccgcctgggcaataccaaggtgcctggcgagttgtcgctgccccagggtgaaacccgagtggaccgcgcgacgatcgaagcaatgccggcgggcaatggcgacatcaccagcctgttgcgcagccatcccaacgtgcagttcgacgacgctcagctgagcagcaagacccctggcgaaatcggcccggccaacatcagcatcaacggggcgcagttctaccagaatgccttcctggtcgatggcatcaacatgaacaatgacatcgatccggccgaataccgggtgcaattgctggacgcggtaccggggcgcagccagggcctggcactggacaccgacctgctggagaacatcacggtcctggacagcaacgtgccagcggcctatggcggcttcaacggtggcgtcatcgatgcacagacccgtgcaccgacccaggcgctgcacggcaaattctcggtgcagaccacccgctccgcctggacccaatatcacgtcgacgaaagcgaagaacaacgcatggaaaactcgggctctcccgacgagcagcccaagttcgacaaggtcaccgttcgtggcacgctgcagggccacctgaccgacaatttcggcctgcttggcagcttcagccagaagcgctcgaccatccccctgggtttctactcctacaacaatgtcgaggagatgggctttcacgaggagaagcagcaacggcgcatcgacaactacttcctcaagtcggtatggaaagccagcgatcaactcactgttgagaccagcgtgacccatgcccccgaggagagtcactacttccgtgccaacgtggccaactcgggttatgacaacaaggccggcggcacccaagtgaacctgcgcaccgtctgggatggcgacctggcgcgcgtcgaacagaacctggcgtggagcggccaggaacagagccgccaatcggactccgccgattactggacctggcgcaagtcggccaccaaggactggggcatcggcaacaaggccacctccaatacccttgaaggcggttggggtgatatcgagcagcagcagcgtacctggcagtacaagctcaatgccgactggcgcagcgtcgactggctgggcatgactcaccgcccgcagaccggcctggagttctcccgccagtacgtgcgctacgagcgcctgaccgacagcagcacctatgtcacccccgcggccaccaccacctgcaccaacggcgccggggttaccgacagcgtcgcctgcaacatcggccagaccctgaacggctgggcgggccagtacatgaaaagccgtacccgctatgccactggcgaattcgacttcaccaccacctcctggggcagctggctgcaggacgagatcaccctcggccaactgaccttgcgccccggcgtgcgcgtggacagcgacgactacatggacaagaccacctgggcgccgcgcttctccctggaatatgacctgttcggtgaccgcaccaccttgctcaatgccggcgtcaaccgttactacgggcgcagcgtcagcagctggcagctgcaggacgggcgcaaccgcctgcgcttcaacgaaacccgcgacagcctggacaaaccctgggtggtcaaatccaacgcggccaaccaggtgcgcttcaatcagctggacatcccctacgacgacgagctgatgctgggcgtggtgcagcaatgggccggggtcgaatatgccctcaagtatgtcaatcgcaaggggcgcgaccaggtggttcaggtgtccggcaagaccttgggcgagccctccacggacccgacgctggcgagcaactacaccacctacaccaacgacggcaagagcgagaccgacacctacaccctgacggtgacaccgttgcagcgctggacactgctgggcacccagaccggcgggcaactggcgctcaactggaccgacagcaaggcctccgcaccgacctacgtcgcggccgagaacctgtactaccagaacagcgtcatccagtaccagggcagcttcatcaattatgacgaccgcccggccagcaacttcaaccggccctggacggcccgcctgacgaccatcaccgagatcccccaggccaatctgcagtggagcaacttctggcgctaccgcgccggctacaagcgcattggcgacacggggcgcaatgtggactacatgggcacctcggtggatgtctgggaggaacagaaattccaggcggcgctgacctgggacacgcgcctgggctggcgtattcccaccgccagggaccagaacgtgttcatcaatgtcgacgtgttcaacgtgctcgacaagaagtcggtcaatgccaaccagacggtcaacagcaactcggtctcgacctatgaggtgggccgccagtactggctcgaagtcggatacgcgttctgaaatgcgccggtccctgaagagcctgcaggtcatgctcggcatggtgctggtgattggcccgcagcacgtgctggcctgcgccacgagcgaaccgtcttgcctgcgcgagttgtacagccgcccccccgcgcagtggccggcgccgcaggtggatgccggcgtggcctggcaagagctgggccccttgcccgagcgggcgccgtccccggcgtacaacccctacacgcagcagaaggccgatctgggccgccggctgttcttcgacccgcgcctgtcgcgttcggggcagattgcctgcgcctcctgccatgaaccggacctggggttcgccgacgggcgtcgggtttcgttcgggcatgaccgtgccgcgggccggcgcaatgcaccgagcctggtggccagcggcctggccaagaagctgttctgggacgggcgcgccgacagcctggagatgcaggccctgatgccggtcgtcgaccccaaggagatggccttcactgtcgcgcaactggtggcccggctgcgtgacaccacggactacccggcacagttcgcccaggtatttcccggacaggcgctgggtgccgagcaggtggcggcggcattggcgacttatcagcgtggcctgctgcgggttgcccaacgcacccccttcgagcgttttctacgggggcaggcgaaggcgctcagcgaccagcaattgcaggggctgcacctgtttcgcaccaaggcacgctgcatgaactgccacttcggcccggggatgcaggacgaccgcttccacaatgcaggcctgaccttctatgggcgcctgcgcgaagacctcgggcgctatgaagtcaccgggctggcgcaggatgtcgggcgcatgcgcacaccttcactgcggctggtgagccataccggcccctggtttcacaacgggctggccagcagcctcgatcaggtgctgttgttctacaacgcaggcatgcccaggcccgtacccaaggaggggcagctgcaagacccgctgtttcccgtgacgtcggcacaattgaaggtattggagctggaccggaccgaactgaaggcactgaaggctttcctggaggccctatgagaggttggccgcggcggctgcacatcggtgtgcccacacagggccagcgccagggacttgatcacctcctgcccgctgcggataccgctgtcgagccagacttcgaggcgctgggcaacaggcgcctggcggcagcgggccggcggctggcgatctaaagagttcgtaaagtctgcccgcccgcccctcccggcacctggcaaatgcgcttcaatagcctgcaagccaagcctgaagctcccggagaagcccatgagcaaagtcgcccgctatctgcaggtcgaagacatcctcctggcggtcaatgtcacgaacaagcagcgcctgttcgagcaggtcggccagcacctgcaagagacccggcaactgccggcggattgggtggccgccagcctgtggcgccgggagctggccgccagtaccgccatcggtgaaggggtggcggtgccccacgcgcgactcgccgagctcgatcgcattcatgcgctgtacctgcggccgcagaccgccatggcctttgccgccccggaccagcagccggtcagcgatatcctggtgctgctggtgcccgctcccgccgatcagcagcacctggacctgctggccgacaccgcccggctgtttaccaacccgcggtttcgccaggccctgctgcgctgtactgcaccgatgcaggtcaaacagctgttcgatcactggtagcttgacccagacacctgctccagcacgcaatacccatttgccgctccccgcggcaaatgggatagcccaagccccaccgatcaccttccggcacaagatttcatctattggcggttagccgactcatccattaacgttcactacacctcacgcagcccagcgcttgaggggggcgcctgccctgccgtcgcgccgctctccatattcctgttgaactcactcactgattgctcaattagagagaatatgaacaagccacagctgctgcctgtagccactggcggccggcgtcctcacagggacgaaggtaccggtcgaaaatcctctgggcacctgtctcttctgttgcttgcccgctcgtccctgaacctgatctgcatcgtagccctcgccagctgcgccgtggggccggatttcagccgcccggaactgagccgggacgccggctacagcgccacgcccctgccgccccgcaccgtcacggcgggggtcgccgccggtggcgcggcgcagcgactggtgaacggcatggacattcccgggcagtggtggacgctgttccgctcgccgcaactcgatgccctggtgcaggaagcgctgcaggccaacccggatatcgacgccgcccaggccgcgctgcgccaggccaatgaactggtgtacgccgaacaggcgtcgctgtttccctccctcagcgcctcggcctcgaaaacccgcgagaaggtgtccgccgccagcgccctgggctccagcggcggtgcggcagcggccgggggctctgcggcgcagatcttcacggtcaactcggcgtccctgagtgtgtcctacgcccccgacgtgtttggcggcacccgccggcagatcgaggccagcggtgcccaggccgactaccagcgctaccagctggaggcgacctacctgaccctcaccgccaacctggtgaacaccgccatcagcctggcctcgatccgtgaccaaatcgccgccaccgaaaccgtgattcgcctgcagagcgaccagctcgacctgctgcaggcccagcgtcgactgggtgccatcggcgacagcgatctgctgacccagcaagcgaccctggcccagacccgcgccaccctgccgcccttgcagaaacagctggcccagacccgcaaccagttgcaggcctacctgggtcgcttccccagccaggaccgtggcgaacacttccagctggcctccctgcacctgcccgaggaactgcccctgagcctgccttcggccatcgtcgagcagcggcccgacgtgcgttccgccgaggcccagttgcatcaggccagcgccgatatcggcgtggccatcgccaaccagttgccgcagttcagcattaccggttccctggggtccacggcgctggcgggcaccaaactgttttcctccggcaccggggtctggagcctggccggctccatcgcccagccgctgttcgacgccggcgccctggagcatcgcaagcgcgccgcggtggccgcctatgagcaggccgcagcgcgctatcgcggcacggtgatcaccgccttccaggacgtagccaatgccctgcgtgccctgcaggccgacgccgaggccctgcaacagcaagtgctggccgagaccgccgcgcgccagagcctggacctggcccaggcccagtaccgcctgggcgcggtgggctacctgaacctgctgaccgcgcagcagacctaccagaacgcgattgtcagccgggtgcgggcccaggccgcccgctacagcgacaccacggcgctgttccaggccctggggggcggctggtggcagcgccaggatgtcgaccccgacagcctcggcagccccgatcgtttcggctggccgtcgctcaaggagatgtacccgccgcccgcggcccagcaccgcgaacaggctcaagccggccagccgggccggctccaggggccggcagcagccgcgccgtccactcgacctgcacccttgccttaagtgagatccgccatggccgatgtccactccagcccctcccctgcgccccccggcgcggccccccaggcgccgcgcaagcgccggctgctgcggccgatgctgatcatgctcggcgtggtgctgctgatcgtcgcggtgatcggcggggtcaagttcgcgcagatctccaagctgatcgcccaggccaaggtgcccctgccgcccgcggtggttaccgccatcaaggcccagtacgaagagtggcagcccagcgtctccgcagtgggctcgatgaagactgtgcgcggggtggacgtgaccaccgaggtcggcggcatcgtgcgcagcatcggcttcaagcccggccaggaagtggccgccggcgacctgctggtgcagttgaatgccgactccgacatcgcccagttgcattccctggaagccaccgcggccctggccggcacggtgctcaagcgcgaccgggcgcaactgaaggtgagcgcggtgtcccaggccctggtcgaggccgacgaagccgacctcaaggccaaactggccgctgccgaacagcagcgcgcgctggtggcgaagaagagcatccgcgcgccctttgccgggcgcatcggcatcaccgcggtcaaccccgggcaatacctcaacccgggggacaagatcgccaacctgcagaccttcgacccgatctacatcgacttcaacgtgccccaggcccaggtgcagcaggtggccctgggccagagcgtcacggtcagcgccgacggcctgcccaaacagacgttcaccggccgggtcagctccatcgacacccagttcgaccccaacacccgcaacgtcacggtggaagccaccatcgacaaccccaagcgcagcctggtgcccggcatgttcgcccgggccgtggtggcctccggcggcacccagcgttacctgaccctgccccagaccgcggtcacctataacccctacggcaccacggtgttcatcgccagccagaagaacaacgaccagggcgacgcagtgctcaccgcgcagcagaccttcatcgaaaccggccccaaccgtggcgaccaggtggcggtgctgtcaggggtcaaggaaggcgacctggtgatcaccagcggccagatgaaactcaagaacggctcgccggtgaaagtcgacaacagccacgcgccgcgcaacgatccttcgccgacgccgcaagagcactagggcccgccatgaacttcaccgatacctttatcaagcgcccggtctgggccgtggtggtctcgctgttcatcctgatcctgggcctgcgctcgatcttcgagctgccggtgaaccagtggccgcgcacggaaaacaccgtggtcacaatcagcacctattactacggggccgacgccgccaccgtggccggtttcattacccagcccctggaggccgccatcgcccaggcccagggcatcgactatctgtcgtccaccagcatcaccgggctctccaccattaccgcgaccctgcgcctgaactacgactccagcaaggcgctgaccgagatcaacacccaggtcaactcggtgaagaaccagttgccggcccagtcccaggaaccggtgctgacggtagcggtgggccagaccaccgacgccatgtacctggggttctacagcgacaccctggccaccaacaacatcaccgactacctggtgcgggtggtcaagcccaagctcgattccctggaaggcgtgcagaccgcagagatcctcggcggccgccagttcgccctgcgggcctggctcgacccggacaagctggcggcccacaacgtcaccgcccaggatgtggccacggccctgggcaacaacaactacctgtccgccgtgggctccaccaaggggcagatgatcaccgtggacctcaccgccggcaccgacctgcacacggtggacgaattcaagcgcctggtggtcaagcacaacggcgacgccctggtgtacctggaggacctgggcaacgtcaccctgggggcggaaaactacgacagcagcgtggctttctccggcaagcgctcggtgttcatcggcatcaaggcagcgcccaccgccaacatcctcaacgtcgccgacaacgtgcgcaaagccttccccgaactgcaatcgcaactgccggccggggtgcgcggcgagatcgtctacgactccacggccttcatcaacacctcgatcttcgaggtggtgaagaccctggtggaagccatgatcatcgtctcggtggtgatctacctgttcctcggctcgttccgtgcggtgatcgtgccgctggtggcgatcccgctgtcgctggtgggcaccttcttcgtcatgtacctgctgggttactccatcaacctgctgaccctgctggccctggtgctggccatcggtcttgtggtggacgacgccatcatcgtggtggaaaacgtcgaccggcatatcaaggagcaaggccgcggcgtactggaggcggcgctgctggcggcccgcgagctgggcggcccgatcatcgccatgaccgtggtgctgatcgccgcctacgtgcccatcggcctgcgcagcggcctgaccggggcgctgttcaaggagttctgtttttccctggccggcgcagtgacggtgtcggcggtgatcgccctgaccctgtcgccaatgatgacctcgcggctgttcaagagcggccaggaggaaggccggttcgccaagcgcctggaccgctacttcgactggctgcgggggcgctaccaccgggtgctggcggccgggctggatagctggccggtgctggtgaccttcggcttcctgctgttcgtgctggtggccgccagtggcctgaccgccaagagcgagctgtcgcccaccgaggaccagggcctggtgttcatgcagatcaagggcgcgcccaccgcctcgccgcagcagatggagtggattgccgaccaggcgttccagatcgccaacaaggagcccgagtacctgcagatgttccagctcaccggcctgccctcgctgaaccagggcctgggcggggtgctgctcaagtcctgggaggatcgtgaccgctcccaggcgcagttgatcctcgacctgcaacagaagtggaaccaggtgcccggcgccaccatcgccgccttccccctgccctcgctgcccggcgcccagggcctgccggtgcagttcgtgatcagcaccaccgagtcggtggccaacctcaacgaggtggcccaggcggtactggccgaggccaccaagcagcagctgttctggttttccgacctggacctcaagctcgacaagccccaggccaagctggtggtggaccgggaaaagatcgccgccctgggcatgacccaggccgacgtcggcgccgcgctctcggcggccctggggggcaactacgtgaactacttctccaccgccgggcgctcctacaaggtgatcccccaggtgctgcaggtggaccggctgaaccccgaacagatcctcgactactacatccgtaccccgtcgggggcgatgattgcagcgcgcaccgtggcccatatcgaaacctccaccgagcctgagtcggtcaaccacttccagcagctcaactcggcgaccctgtccggggtcagcggcgtgtcccagggcgagttgctggcccggctcaagggcattctggaacaggtggcgccatccggttactccagcgactacgccggcgaatcacggcagttcatccaggaatcgggggggttcgtcggcctgctgctgttctcgatcctgatcgtctacctggccctggcgttccagttcgagagctaccgcgacccggtggtgatcctgttctcggtgccgccggcgctgttcggcgccctggccttcatcaccatggggttcgcctcgatcaacgtctacacccaggtcgggctggtgacgctgctggggctgatcaccaagcacgggatcctcatcgtgcagtttgccaaccaattgcagcgcgccggccacaataagcgcgaagccatcgaagaagccgccgcagtgcgcctgcggccgatcctgatgaccaccgcggccatggtcctgggggtggtgccgctggtctgggcttccggcgccggcgcggcgggccgccacgacatgggcctggtgatctttgccgggctgtccatcggcaccctgctgaccctgttcatggtgccggccatgtacctgttcattggcagtacgcactctgccgcaagcatcgagaacccgggggcgcaggcggatgccggagcttccgacagcgcctgaacccctgctcgatcgaaaccgccgggtatcaggccggcacgcccaggatgatgtgggacgccttgatcaccgccgtggcgctgacgcccggggccaggcccagttcggtcaccgcatcgcgggtgacgatggaatacacctggctgccgccgctgagctccagcaccacttcggcgttgaccgcaccatcggtgacgctgatcaccttgccccgcaggcagttgcgagcggacaggcggatgccctcggcatcggtcatgagcatcacccagggtgccttgaccagggccagggcctcggtgcccggggcgatgcccaggctacgcaggctttgcagggtcaccaccgccacaagttgctcgccgccctccagggacagcaccacctcggcgttgaccgaaccctcttccacctggctgacccggcctttgaacacgttgcgtgcactgactttcatgttgcattccttttattgactgcggggttaggatcgcccggcatgatccgggcgcacgttgtggagaaacaaccatagcgcccgccacccaaggccggatatttctcgcaggccccgggtcaatcctgctcttgcaggtgttcgataaaggtcgtgccgctatagcgctcgcgaaacagccccgcctccaccagcatcggtatcacctgttccaggaaaatgtgcatcgaacggatcgagccccccggcgcggcgataaagccgtccatggcgccggcctcggcccattcgacgatctgtgcaaatgcatcttcggcggtgcctatcacttgccagtgggccgcggaaatcacttccgggcgcagcagcagttcgtccaggcgcagggtttcgcgctcgatcaaccgccgcagcaggttggtgtgggtgcggctgccggggttgtgcaccggggccggcaggtcggcggcgctcaccgggcggtccaggggccagtcgcttaaatcgaggccgatcatctgctggatcgaggcgaacttgcgcgcgttgtccgggcgcccgtgggtttgcatgaacagctcgcgggcctgttcccgggtcggcgccaggtacaggctcaggcccggcagcaggcggatgtcctcggggcggcggccgtggcgccgggcccgttgcgagaggtcgcgacgcagttccagggccgcatccttgtccggggtcggggcgaacaccaggtcggccaccgaggcggcaaagtcgcgaccggtgtccgaggcgccggcctggaccaggggaatccgtgccttgccgaacgccggcaggttgagcgggccgcgcacccggaagtattcgccggcatggtccaccgggtgcacctggctgcggtcggcgtaacggccgttctcccggtccagtagcagcgcctggttggggtagctggcccacaggtcgtgcaccaggcgggtgaactctgccgcccgggcatagcgctcatccgcgccgggcatggcctccaggccgaagttctcgtggccctgcaaggcggtgacgatgttccagccggcgcggccgttgctcacccaatgcagcgactgcaactggcgcgccaccacatagggcgggaagaaggtggtggaaacggtggacaccaggccgatgcgggacgtctcccgggcgatgcacgccagcagcaccgtgggatcgaggctggcgaaacccgagccgctttccaggccgtccatgttcaggcaactgacgtcggggcggaacacgaaatccaggtgcgcggcctcggaacgcttggcgatgtccacggcgaagtcggcgctgtagatgccttcgatattgctgtcgggctggcgccaggcatcgccactgagccaggtgggggccagggacatgccgatatgcaggcgtttgcggttactcatctgttgccaatctcctgtgtacgagtgaacgccggcagcgcctcggggggcggccggcgacgggttcccgggggcctcagaacgcccctttgacgccgatgcccagggtccgtggctgggtcatgctggcctcgatgccgccgatgccgcggttctgctgcatgtaggtgggcgagcggtcgtcgaagacgttcttgacgtagccatagagctgcacctggtcgttgaagcggtagctcatgcgcgcatcggtcagggtgtagggcttgatcgaataggccgaggtgttggcggtatccgagtagtagccgtccaggtagcg

SEQ ID NO: 4 - DNA, 20 bp, synthetic construct
  misc_feature 1..20: note = primer for 16S rRNA amplification
  source 1..20: mol_type = other DNA
agagtttgatcctggctcag

SEQ ID NO: 5 - DNA, 19 bp, synthetic construct
  misc_feature 1..19: note = primer for 16S rRNA amplification
  source 1..19: mol_type = other DNA
ggttaccttgttacgactt

(19) United States
(12) Patent Application Publication
(10) Pub. No.: US 2024/0409953 A1
(43) Pub. Date: Dec. 12, 2024

(54) PSEUDOMONAS PROTEGENS AND PRODUCTS THEREOF TO CONTROL BACTERIAL PANICLE BLIGHT OF RICE

(71) Applicant: THE BOARD OF TRUSTEES OF THE UNIVERSITY OF ARKANSAS, Little Rock, AR (US)

(72) Inventors: Clemencia M. Rojas, Fayetteville, AR (US); J. Alejandro Rojas, Springdale, AR (US); Jackson O. Lay, JR., Fayetteville, AR (US)

(73) Assignee: THE BOARD OF TRUSTEES OF THE UNIVERSITY OF ARKANSAS, Little Rock, AR (US)

(21) Appl. No.: 18/652,391

(22) Filed: May 1, 2024

Related U.S. Application Data
(62) Division of application No. 17/341,498, filed on Jun. 8, 2021, now Pat. No. 11,999,963.
(60) Provisional application No. 63/036,202, filed on Jun. 8, 2020.

(51) Int. Cl. C12N 15/82

(52) U.S. Cl. CPC C12N 15/8281

(57) ABSTRACT

The present invention provides biosynthetic products that may be useful for controlling bacterial panicle blight of rice. These products are encoded by four biosynthetic operons found within the genome of a Pseudomonas protegens strain designated as PBL3 and deposited as ATCC #, which has been shown to inhibit the growth of Burkholderia glumae on rice plants. Also provided are vectors encoding these biosynthetic products, and methods of using the biosynthetic products to inhibit the growth of microorganisms.

### CROSS-REFERENCE TO RELATED APPLICATIONS

This application is a divisional of U.S. patent application Ser. No. 17/341,498, filed on Jun. 8, 2021, and issued as U.S. Pat. No. 11,999,963 on Jun. 4, 2024, which claims priority to U.S. Provisional Application No. 63/036,202, filed on Jun. 8, 2020, the contents of which are incorporated by reference in their entireties.

### STATEMENT REGARDING FEDERALLY SPONSORED RESEARCH

NR

### SEQUENCE LISTING

This application includes a sequence listing in XML format titled “169946.00754_ST26.xml”, which is 194,481 bytes in size and was created on May 1, 2024. The sequence listing is electronically submitted with this application via Patent Center and is incorporated herein by reference in its entirety.

### BACKGROUND

Bacterial panicle blight (BPB) of rice, which is caused by the bacterium Burkholderia glumae, is emerging as a serious threat to rice production, especially as global temperatures increase (Ham et al., 2011, Shew et al., 2019). Since its identification in 1967 in Japan, this disease has spread throughout the rice growing areas of the world causing major yield losses (Nandakumar et al., 2007, Nandakumar et al., 2009, Zhou, 2014, Mondal et al., 2015, Shew et al., 2019). Burkholderia glumae causes rotting of the seeds during germination as well as stunting and chlorosis in seedlings (Iiyama et al., 1995). In older plants, symptoms can appear in leaves and stems, and are characterized by gray lesions surrounded by brown margins (Nandakumar et al., 2009). During the reproductive stages, the bacterium infects reproductive tissues affecting grain development and causing the main symptoms of the disease in the panicle, which include discolored spikelets with stained, rotten, or unfilled kernels (Nandakumar et al., 2009, Wamishe et al., 2015). The effects of the disease on grain development are reflected in a reduction in grain weight of up to 75% and seed abortion with concomitant yield losses (Fory et al., 2014). The disease symptoms are mostly associated with the production of the toxin toxoflavin, which is considered the main virulence factor (Iiyama et al., 1995, Karki et al., 2012). While other virulence factors have been identified by homology with virulence factors from other phytopathogenic bacteria (Devescovi et al., 2007, Degrassi et al., 2008, Kang et al., 2008, Lim et al., 2009, Fory et al., 2014, Knapp et al., 2015), their contribution to pathogenesis and virulence is not clear.

Burkholderia glumae is transmitted through contaminated seeds. Thus, the use of pathogen-free seeds as well as cultural practices such as early planting, reduction in nitrogen fertilizer, and reduction in planting density can mitigate the effects of this disease (Wamishe et al., 2015). Chemical control using the quinolone antibiotic oxolinic acid has been restricted to Asia (Hikichi, 1993, Lee et al., 2015), but it is not very effective due to the development of resistance by the pathogen (Maeda et al., 2004). To date, there are no commercially available rice cultivars that show complete resistance to B. glumae, but it would be possible to develop them given that quantitative trait loci (QTLs) associated with resistance to the disease have been identified (Mizobuchi et al., 2013, Mizobuchi et al., 2016). In addition, high levels of resistance against B. glumae have been achieved by overexpression of BSRI (Broad-Spectrum Resistance 1), a gene encoding a receptor-like kinase (Dubouzet et al., 2011, Maeda et al., 2016). Even with these promising findings, the development of completely resistant cultivars will take several years. Thus, there remains a need in the art for alternative methods to control bacterial panicle blight of rice.

### SUMMARY

In a first aspect, the present invention provides biosynthetic products produced by a Pseudomonas protegens strain designated as PBL3 and deposited as NRRL Accession No. B-68083. The biosynthetic products are selected from orfamide A/C, pyoluteorin, and pyrrolnitrin.

In a second aspect, the present invention provides vectors encoding gene products required for the synthesis of the biosynthetic products described herein. The vectors comprise a heterologous promoter operably linked to at least one open reading frame (ORF) found within SEQ ID NO:1, SEQ ID NO:2, or SEQ ID NO:3, which are the sequences of the gene clusters responsible for the synthesis of orfamide A/C, pyoluteorin, and pyrrolnitrin, respectively.

In a third aspect, the present invention provides organisms comprising the vectors described herein.

In a fourth aspect, the present invention provides methods of inhibiting the growth of a microorganism. In one embodiment, the methods comprise contacting the microorganism with an effective amount of a biosynthetic product described herein. In a second embodiments, the methods comprise contacting the microorganism with an organism comprising a vector described herein or a biosynthetic product produced by the organism.

### BRIEF DESCRIPTION OF THE DRAWINGS

FIG. 1 shows the identification of bacteria with antagonistic activities against Burkholderia glumae. Burkholderia glumae was mixed with molten KB agar to a final OD₆₀₀=0.001 and poured into plates. After agar solidified, five sterile filter paper disks were placed on the agar surface. Overnight cultures of seven bacterial strains: P. protegens PBL3, P. fluorescens 5-40, P. fluorescens 1-30, B. cepacia, P. fluorescens sp. P. fluorescens 2-79 and Burkholderia sp. were diluted to OD₆₀₀=1 (1×10⁸ CFU/ml), and 5 μl of bacteria were pipetted onto four of the filter paper disks. Water was added to the fifth center disk and used as negative control. Plates were incubated at 28° C. for 48 h. The clear zones of growth inhibition (A) were measured and plotted (B). Bars represent the average area of B. glumae growth inhibition by different bacteria from replicates of three independent experiments. Each error bar indicates the standard error from replications. Treatments were compared using ANOVA and means were compared using Tukey honestly significant difference (HSD) test with P-value=0.05.

FIG. 2 demonstrates that co-inoculation of B. glumae with P. protegens PBL3 reduces disease symptoms in rice. Eight-week-old rice plants from cultivar Wells were mock-treated with water or inoculated in the sheath with B. glumae diluted to OD₆₀₀=0.125 (1×10⁷ CFU/ml), or in combination with P. protegens PBL3 or E. coli, each diluted to OD₆₀₀=0.125. Disease symptoms were evaluated after eight days (A). Bars represent average lesion lengths (B). Each error bar indicates the standard deviation from replications of three independent experiments. Treatments were compared using ANOVA and means were compared using Tukey's HSD with P-value=0.001.

FIG. 3 demonstrates that Pseudomonas protegens PBL3 encodes biosynthetic operons for orfamide A/C, pyoluteorin, and pyrrolnitrin. The Pseudomonas protegens PBL3 genome was sequenced by Illumina. Illumina reads were assembled using Shovill (github.com/tseemann/shovill) and compared with the genome of the reference strain P. protegens CHA0 (NCBI CP003190) using anti-SMASH. Colored arrows represent individual open reading frames (ORFs) within gene clusters associated with the biosynthesis of orfamide A/C, pyoluteorin, and pyrrolnitrin. The sequences for the genes associated with each of these operons are attached and submitted herewith as SEQ ID NOs: 1-3.

FIG. 4 demonstrates that the secreted fraction from P. protegens PBL3 interferes with the growth of B. glumae. LB broth and the secreted fraction from P. protegens PBL3 were lyophilized, resuspended in sterile water to a final concentration of 0.1 g/ml, and filter-sterilized. Resuspended LB or resuspended secreted fraction from P. protegens PBL3 were mixed in a 1:1 ratio (V/V) with KB broth. A single B. glumae colony was added to these tubes and grown for 24 h at 28° C. with agitation. After 24 h of incubation, growth of B. glumae was visually evaluated (A), and aliquots were taken, serially diluted, and plated to count the number of bacteria on KB plates (B). Bars represent the average growth of B. glumae (in CFU/ml) in both conditions. Each error bar indicates the standard deviation from replicates from three independent experiments. Asterisk denotes statistically significant difference using T-test with a P-value=0.05.

FIG. 5 demonstrates that the secreted fraction from P. protegens PBL3 reduces disease symptoms caused by B. glumae. Lyophilized LB and secreted fraction from P. protegens PBL3 were resuspended in sterile water at a final concentration of 0.5 g/ml, filter sterilized, and mixed in equal volume with B. glumae at OD₆₀₀=0.125 (1×10⁷ CFU/ml). Twenty microliters of the respective inocula were injected onto the sheaths of ten-week old Nipponbare rice plants (A-D). Inoculated sheaths were used to evaluate disease symptoms and to quantify bacterial growth at 2 dpi (A and B) and 7 dpi (C and D). Bars in B and D represent average bacterial numbers (Log CFU/in). Each error bar indicates the standard deviation from replicates of three independent experiments. Asterisk in B denotes statistically significant difference using T-test with a P-value=0.05.

FIG. 6 demonstrates that the secreted fractions from P. protegens PBL3 reduces disease in seedlings. B. glumae at OD₆₀₀=0.001 (1×10⁵ CFU/ml), was mixed with lyophilized and resuspended LB at 0.5 g/ml, or lyophilized and resuspended secreted fraction of P. protegens PBL3 at 0.1 g/ml or 0.5 g/ml. Sterile water was used for the control. Twenty sterile seeds of cultivar Nipponbare were incubated with either B. glumae mixed with resuspended LB or with B. glumae mixed with the resuspended secreted fraction of P. protegens PBL3 for 30 min. Inoculated seeds were plated on MS plates and incubated for 2 days at 28° C. and then transferred to a growth chamber 25° C. under long day conditions (16 h light/8 h dark) for five days. Shoot length of rice seedlings was measured 7 dpi (A and B). Bars represent average shoot length. Each error bar indicates the standard deviation from replicates from three independent experiments. Letters above bars represent statistical significance using ANOVA and comparing means with Tukey's HSD tests with a P-value of =0.05.

FIG. 7 demonstrates that Pseudomonas protegens PBL3 antimicrobial activities against B. glumae are related to molecules of different molecular weights. The lyophilized secreted fraction of P. protegens was reconstituted in sterile water and concentrated with ultrafiltration centrifugal concentrators of different molecular weight cut-offs: 30 kDa (A), 10 kDa (B) and 3 kDa (C). Sub-fractions higher and lower than the nominal molecular weight cut-off were filter sterilized and mixed (V/V) with KB broth. Water was also mixed with KB broth and used as control. KB amended with the different sub-fractions preparations was used to grow a single colony of B. glumae at 28° C. with constant agitation for 18 h. Cultures were serially diluted and plated on KB agar to enumerate bacterial populations (in Log CFU/ml). Bars represent average and standard deviation of bacterial growth for three replications. Different letters above the bars indicate statistically significant difference among treatments based on ANOVA and Tukey's HSD test with a P-value of 0.05.

FIG. 8 shows the effect of NaCl on the growth of B. glumae. Different concentrations of NaCl (0.1M, 0.5 M and 1M) were added to KB broth in a 1:1 v/v ratio and used to grow a single colony of B. glumae at 28° C. for 22 h with constant agitation. After 22 h, a bacterial aliquot of each treatment was serially diluted and plated on KB agar to enumerate bacterial populations. Bars represent average and standard deviation of three replications. Letters above bars represent statistical significance using ANOVA and Tukey's HSD tests with a P-value of =0.05.

FIG. 9 demonstrates that the organic phase of the P. protegens PBL3 secreted fraction does not contain antimicrobial activity. The secreted fraction of P. protegens PBL3 was lyophilized and resuspended in water at a final concentration of 0.1 g/m. Fifteen milliliters of the resuspended fraction were mixed with 15 ml of hexane, transferred to a separatory funnel, and mixed by inversion five times. After phase separation, the aqueous phase was collected in one beaker, and the organic phase was collected in another beaker. The organic phase was set aside, and the aqueous phase was once again added to the separatory funnel and other 15 ml of hexane were added to the separatory funnel and again separated into aqueous and organic phases. This process was repeated one more time for a total of three times. The hexane layer was then blown down to dryness with nitrogen gas and resuspended in 3 ml of water. An aliquot of each of the phases were mixed with KB broth in a 1:1 v/v and used to culture a single colony of B. glumae by incubation at 28° C. in a shaker incubator for 22 h. Fractions were then serially diluted and plated on KB agar to count the number of bacteria (in CFU/ml). Bars represent average bacterial growth for three replications. Asterisk denotes statistically significant difference among treatments using ANOVA and Tukey's HSD tests with a P-value of =0.05.

FIG. 10 demonstrates that the antimicrobial activity of the secreted fraction of P. protegens PBL3 is retained after boiling and protease treatment. LB and the secreted fraction of P. protegens PBL3 were lyophilized and resuspended in water to a final concentration of 0.1 g/ml. Two hundred microliters of resuspended LB and 200 μl of the resuspended secreted fraction from P. protegens PBL3 were boiled in water for 5 min. After removal from heat, samples were mixed with 200 μL of KB broth. As controls, 200 μl of non-boiled LB and 200 μl of non-boiled secreted fraction from P. protegens PBL3 were also added to 200 μl of KB. All these amended KB broths were used to grow a single colony of B. glumae at 28° C. for 22 h with constant agitation. After 22 h, bacteria were serially diluted and plated on KB agar to enumerate bacterial populations. In addition, 200 μL of the non-boiled secreted fraction from P. protegens PBL3 was mixed with 10 μL of Proteinase K and incubated at 37° C. for 30 min. Protease was deactivated by heating it at 65° C. for 10 min. The protease-treated secreted fraction of P. protegens PBL3 were added to KB broth in a 1:1 v/v ratio and used to grow a single colony of B. glumae at 28° C. for 22 h, as previously described. After 22 h, bacteria were serially diluted and plated on KB agar to enumerate bacterial populations. Bars represent average and standard deviation of bacterial growth (in Log CFU/ml) for three replications. Different letters above bars represent statistically significant difference among treatments using ANOVA and Tukey's HSD tests with a P-value of =0.05.

### DETAILED DESCRIPTION

Biological control using microorganisms that compete or have antagonistic activities against B. glumae is one promising approach to control bacterial panicle blight of rice. Previously, several strains of Bacillus methylotrophicus, Bacillus amyloliquefaciens, Bacillus subtilis, and Streptomyces sp. with antagonistic activity against B. glumae were identified in vitro (Shrestha et al., 2016, Suarez-Moreno et al., 2019). Five of the B. amyloliquefaciens strains also caused a reduction of disease symptoms when plants were pre-treated with these antagonistic bacteria before inoculation with B. glumae, but follow-up experiments in the field were inconsistent (Shrestha et al., 2016).

The present application is based on the inventors' discovery of a strain of Pseudomonas protegens that inhibits the growth of B. glumae. As is described in the Examples, the inventors evaluated the potential of several bacteria to control bacterial panicle blight. They tested seven bacterial strains that were previously investigated for their biological control activities against the oomycete pathogen Pythium sp. (Milus & Rothrock, 1997). The inventors identified one strain of Pseudomonas protegens, designated as PBL3 and deposited as NRRL Accession No. B-68083, which inhibited the growth of B. glumae in vitro and reduced disease symptoms in rice when co-inoculated with B. glumae. Further, they demonstrated that these activities were associated with the cell-free secreted fraction of PBL3. These findings suggest that the bioactive compounds produced by this bacterium can be used to control bacterial panicle blight of rice.

Accordingly, the present invention provides biosynthetic products (i.e., orfamide A/C, pyoluteorin, and pyrrolnitrin) that may be useful for controlling bacterial panicle blight of rice. Also provided are vectors encoding proteins involved in the synthesis of these biosynthetic products, organisms comprising these vectors, and methods of using the organisms and biosynthetic products to inhibit the growth of a microorganism.

### Biosynthetic Products

In a first aspect, the present invention provides biosynthetic products produced by a Pseudomonas protegens strain designated as PBL3 and deposited as NRRL Accession No. B-68083. The biosynthetic products are selected from orfamide A/C, pyoluteorin, and pyrrolnitrin.

As used herein, the term “biosynthetic product” refers to a product produced by a living organism. The biosynthetic products of the present invention are secondary metabolites that are responsible for antifungal activities in the closely related reference strain P. protegens CHA0. Thus, one or more of these products may be responsible for the anti-B. glumae activity that is present in the secreted fraction of P. protegens PBL3. The biosynthetic product may be combined with other products in an agricultural composition. The other products included in an agricultural composition may include a fungicide, an herbicide, an insecticide, a biosanitizer product, or a fertilizer.

Orfamides are lipopeptide biosurfactants that are produced by Pseudomonas and are involved in lysis of oomycete zoospores, biocontrol of Rhizoctonia and Magnaporthe oryzae, and insecticidal activity against aphids. Pyoluteorin is an antibiotic that inhibits Oomycetes, including the plant pathogen Pythium ultimum, and suppresses plant diseases caused by this oomycete. Pyoluteorin is synthesized from a hybrid nonribosomal peptide synthetase or from the polyketide synthase pathway, depending on the producer strain. Pyrrolnitrin is a pyrrole antifungal agent that has been isolated from several Pseudomonas, Serratia, and Burkholderia species. Pyrrolnitrin has a broad-spectrum of activity and can be used for biological control of soil and seed-borne plant diseases.

Orfamide A/C, pyoluteorin, and pyrrolnitrin are produced by four biosynthetic gene clusters that were identified within the genome of PBL3. The DNA sequences of these gene clusters are provided herein as SEQ ID NO:1, which encodes gene products necessary for the synthesis of orfamide A/C; SEQ ID NO:2, which encodes gene products necessary for the synthesis of pyoluteorin; and SEQ ID NO:3, which encodes gene products necessary for the synthesis of pyrrolnitrin.

### Vectors

In a second aspect, the present invention provides vectors encoding gene products required for the synthesis of the biosynthetic products described herein. The vectors comprise a heterologous promoter operably linked to at least one open reading frame (ORF) found within SEQ ID NO:1, SEQ ID NO:2, or SEQ ID NO:3, which are the sequences of the gene clusters responsible for the synthesis of orfamide A/C, pyoluteorin, and pyrrolnitrin, respectively.

In some embodiments, the at least one ORF included in the vector encodes a gene product that is involved in the synthesis of orfamide A/C, pyoluteorin, or pyrrolnitrin. As used herein, the term “gene product” refers to a protein or functional RNA that is encoded by a gene. Thus, the vectors of the present invention can be used to produce gene products that are necessary to synthesize the biosynthetic products disclosed herein.

In some embodiments, the vectors comprise a plurality of ORFs found within SEQ ID NO: 1, SEQ ID NO:2, or SEQ ID NO:3. In some embodiments, the vectors comprise a full-length gene cluster selected from SEQ ID NO:1, SEQ ID NO:2, or SEQ ID NO:3.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”. Vectors suitable for use with the present invention comprise one or more ORF described herein and heterogeneous sequence (i.e., sequence from a difference species than the polypeptide) necessary for proper propagation of the vector and expression of the encoded gene product.

As used herein, the term “promoter” refers to a DNA sequence capable of controlling the expression of a sequence to which the promoter is operably linked. In contrast to native promoters that are derived in their entirety from the corresponding native gene, “heterologous promoters” are derived from a gene that is completely separate from gene to which they are operably linked. Heterologous promoters may be composed of multiple promoters elements derived from different promoters found in nature, or may even comprise synthetic DNA segments. It is understood by those skilled in the art that different promoters may direct the expression of a gene in different tissues or cell types, or at different stages of development, or in response to different environmental conditions.

An “open reading frame (ORF)” is a part of a gene that can be translated. ORFs comprise continuous stretches of DNA that typically begin with a start codon (usually ATG) and end at a stop codon (usually TAA, TAG or TGA).

### Organisms

In a third aspect, the present invention provides organisms comprising the vectors described herein. The organisms can be used to produce gene products encoded by the vectors, i.e., by expressing the gene product(s) encoded by the at least one ORF included in the vector. Thus, for clarity, these organisms are also referred to herein as “producer organisms”. Any organism that is suitable for heterologous protein expression may be used with the present invention. Exemplary organisms include, for example, Escherichia coli, Coryne bacterium, Pseudomonas fluorescens, yeasts (e.g., S. cerevisiae or Pichia pastoris), filamentous fungi (e.g., Aspergillus or Trichoderma,) and plants (e.g., Nicotiana sp.). In some embodiments, the organism is E. coli.

### Methods of Inhibiting Microorganism Growth

In a fourth aspect, the present invention provides methods of inhibiting the growth of a microorganism. In one embodiment, the methods comprise contacting the microorganism with an effective amount of a biosynthetic product described herein. In a second embodiments, the methods comprise contacting the microorganism with an organism comprising a vector described herein (i.e., a producer organism) or a biosynthetic product produced by the organism. Thus, the methods of the present invention inhibit the growth of a microorganism by contacting it with a composition comprising (1) a biosynthetic product produced by P. protegens PBL3, as described in the section titled “Biosynthetic products”; (2) a producer organism, as described in the section titled “Organisms”; or (3) a biosynthetic product produced by a producer organism.

The growth of any microorganism may be inhibited by the methods of the present invention. Suitably, the microorganism is a bacterium or fungus. In the Examples, the bacterial strain P. protegens PBL3 and cell-free secreted fractions thereof were only tested against the rice pathogen Burkholderia glumae. However, in previous work, the inventors have shown that this strain inhibits the growth of several additional plant pathogens, including bacterial pathogens from the genera Burkholderia, Xanthomonas, and Erwinia, as well as fungal pathogens from the genera Rhizoctonia, Pythium, Magnaporthe, and Fusarium. See US Patent Application No. 2020/0068900 (note: PBL3 is referred to as Pseudomonas fluorescens PBL 13 in this application, but was later reclassified based on 16S rRNA sequencing, as is described in the Examples). Thus, in some embodiments, the microorganism is a bacterium of the genera Burkholderia, Xanthomonas, or Erwinia, or a fungus of the genera Rhizoctonia, Pythium, Magnaporthe, or Fusarium. In preferred embodiments, the microorganism is Burkholderia glumae.

Inhibition of growth of a microorganism may be assessed using any method known in the art. Suitable methods include, for example, plate inhibition assays. In embodiments in which the microorganism is a plant pathogen, the biosynthetic product or producer organism can applied to the plant (e.g., injected into the plant or applied to the seeds of the plant), and the growth of the microorganism on the plant or the microbial damage to the plant can be measured.

“Effective amount” is intended to mean an amount of a biosynthetic product or producer organism described herein that is sufficient to inhibit the growth of a microorganism by, for example, 10%, 20%, 50%, 75%, 80%, 90%, 95%, or 1-fold, 3-fold, 5-fold, 10-fold, 20-fold, or more compared to a negative control that does not comprise the biosynthetic product or producer organism.

In some embodiments, the methods are used to inhibit the growth of a microorganism that is on a plant. As used herein, a “plant” includes any portion of the plant including, without limitation, a whole plant or a portion of a plant such as a part of a root, leaf, stem, seed, pod, flower, cell, tissue plant germplasm, asexual propagate, or any progeny thereof. For example, a rice plant refers to the whole rice plant or portions thereof including, without limitation, the leaves, roots, seeds or otherwise. Suitable “plants” may include, without limitation, rice, tomato, onion, cotton, soybean, wheat, ryegrass, crucifers, prunus, beans, kiwi fruit, mango, apple, pear, sunflower, maple, European horse chestnut, Indian horse chestnut, beet, hazelnut, barley, cucumber, cabbage, mulberry, cherry, millet, pea, olive, tobacco, camellia, sorghum, or corn. In some embodiments, the plant is a rice plant.

Several methods of “contacting” may be used to apply a biosynthetic product or producer organism described herein to a plant. Suitable application methods include, without limitation, spraying or dusting. Contacting may also be carried out indirectly via application, for example, to the soil surrounding a plant or to plant media or substrates. Alternatively, the biosynthetic product or producer organism may be injected into the plant. The contacting step of the present methods may be carried out before or after the microorganism grows on the plant. In some embodiments, the leaves or seeds of the plant are contacted with the biosynthetic product or producer organism. In some embodiments, the contacting is carried out before flowering or during panicle formation.

In some embodiments, the plant may be contacted at least 2, 3, 4, 5, or more times with a biosynthetic product or producer organism described herein. For example, the seeds of the plant could be treated with the biosynthetic product or producer organism prior to planting and then the biosynthetic product or producer organism could be sprayed onto the growing plants at one or more stage of development. The methods may be used as a preventative measure or may be used only on plants or in fields that microbial damage is suspected or noted.

In methods in which the microorganism is on a plant, the biosynthetic product or producer organism may be applied as part of an agricultural composition. An “agricultural composition” is a composition formulated for application to a plant or plant part. Agricultural compositions are commonly formulated as a liquid (i.e., liquid suspension) for application by spraying or soaking, but may also be formulated in a solid, granular, or powder form for rehydration or application by dusting or dry coating. The agricultural composition may be concentrated (e.g., by lyophilization) for dilution in water or another solvent. The agricultural compositions may be prepared for administration to plants or may be prepared for administration to seeds. The agricultural compositions may include a biosynthetic product or producer organism described herein and a carrier. As used herein, a “carrier” may be solid or liquid and may include substances ordinarily employed in formulations applied to plants. Suitable carriers include buffers, water, oils, nonionic surfactants, ionic surfactants, or agricultural by-products. In some embodiments, the agricultural compositions also include an additional active ingredient.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

Bacterial panicle blight of rice is a seed-borne disease caused by the bacterium Burkholderia glumae. This disease has affected rice production worldwide and its effects are likely to become more devastating with the continuous increase in global temperatures, especially during the growing season. The bacterium can cause disease symptoms in different tissues and at different developmental stages. In reproductive stages, the bacterium interferes with grain development in the panicles and, as a result directly affects rice yield. Currently, there are no methods to control the disease as chemical control is not effective and completely resistant cultivars are not available. Thus, a promising approach is the use of antagonistic microorganisms. In the following Example, the inventors identified one strain of Pseudomonas protegens and one strain of Burkholderia cepacia with antimicrobial activity against B. glumae in vitro and in planta. They characterized the antimicrobial activity of P. protegens and found that this activity is associated with bacterial secretions. Cell-free secretions from P. protegens inhibited the growth of B. glumae in vitro and prevented B. glumae from causing disease in rice. Although, the specific molecule(s) associated with these activities have not been identified, these findings suggest that the secreted fractions from P. protegens could be harnessed as biopesticides to control bacterial panicle blight of rice.

### Materials and Methods

### Bacterial Strains

Burkholderia glumae was isolated from infected rice panicles in fields of Arkansas (Mulaw et al., 2018), and grown on a modified CCNT media (Kawaradani et al., 2000) (2 g/L yeast extract, 1 g/L proteose peptone, 4 g/L Myo-inositol, 10 mg/L Cetrimide, 18 g/L agar, pH 4.8) or King's B (KB) media (King et al., 1954), depending on the experiment. Other bacterial strains used in this study are listed in Table 1. These strains were grown on Luria Bertani (LB) (Bertani, 1951) or KB media, depending on the experiment.

TABLE 1
Bacterial strains used in this study

| Strain | Source |
|---|---|
| Burkholderia glumae | Y. Wamishe |
| P. fluorescens PFS JA 4092/P. protegens | (Milus & Rothrock, 1997)/This |
| PBL3 | study |
| P. fluorescens 5-40 | (Milus & Rothrock, 1997) |
| P. fluorescens 1-30 10-99 | (Milus & Rothrock, 1997) |
| B. cepacia | C. Rothrock, unpublished |
| Pseudomonas sp. | C. Rothrock, unpublished |
| P. fluorescens 2-79 | D. M. Weller |
| Burkholderia sp. | C. Rothrock, unpublished |
| Escherichia coli DH5α | (Hanahan, 1983) |

Bacterial identification was confirmed by the Polymerase chain reaction (PCR) of the 16S rRNA region using primer 27F (5′-AGAGTTTGATCCTGGCTCAG-3′; SEQ ID NO:4) and 1492R (5′-GGTTACCTTGTTACGACTT-3′; SEQ ID NO:5) (Lane, 1991). The reaction mix consisted of 2 μL of template DNA, 2 mM MgCl2, 0.2 mM dNTPs, 0.2 mg/ml Bovine Serum Albumin, 0.2 μM of forward and reverse primers, 1X buffer and 1 U of GoTaq polymerase (Promega, WI, USA). The amplification conditions included an initial denaturation at 94° C. for 5 min, followed by 30 cycles consisting 1 min at 94° C., 1 min at 45° C., and 2 min at 72° C., and a final extension for 10 min at 72° C. The PCR product was visualized in a 1% agarose gel stained using Gel Red® Nucleic Acid Gel Stain (Biotium, CA, USA) and then purified with the GeneJet PCR purification kit (Thermo Fisher Scientific, Waltham, MA). Purified PCR product was sequenced at Macrogen, Inc. (Rockville, MD). Forward and reverse sequences were assembled using SeqMan (DNAStar, Madison, WI), and compared with 16S rRNA bacterial sequences available in The Ribosomal Database Project (RDP) (Cole et al., 2014).

### Rice Seed Sterilization, Germination, and Fertilization

Rice cultivars used in this study include the long grain cultivar Wells and the short-grain cultivar Nipponbare, both of which are moderately susceptible to bacterial panicle blight (Temesgen et al., 2018, Mizobuchi et al., 2018). Rice seeds were de-husked and surface sterilized with 70% ethanol for 30 seconds, followed by shaking for 15 min in a solution containing 30% bleach and 20% sodium dodecyl sulfate (SDS). The bleach/SDS treatment was repeated one more time. After decanting the bleach/SDS solution, seeds were rinsed with sterile water five times. Surface-sterilized rice seeds were placed on sterile filter paper in petri plates with enough water to saturate the filter paper. Sterilized seeds were pre-germinated at 28° C. in the dark for 4-5 days. Seedlings were transferred onto Pro Mix potting soil and transplanted when they were 2-3 inches tall. All Purpose Plant Food Miracle-Gro (Scotts Miracle-Gro, Marysville, OH) was added to fertilize plants after transplanting and every week afterwards. During vegetative growth, plants were also supplemented with iron (Sulfate Sprint 330 10% iron chelate, BASF, Germany) at 0.473 lbs/gal, twice a week.

Screening for Antagonistic Activity Against B. glumae

Overnight liquid cultures of P. fluorescens PFS JA 4092 (herein renamed as P. protegens PBL3), P. fluorescens 5-40, P. fluorescens 1-30, B. cepacia, Pseudomonas sp, P. fluorescens 2-79, Burkholderia sp., and B. glumae grown in KB were collected by centrifugation at 6,000 rpm for 10 min and bacterial pellets were washed with sterile water three times. The concentrations of bacterial strains to be tested as biocontrol agents were adjusted to an optical density at 600 nm (OD₆₀₀) of 1 (1×10⁸ CFU/ml). B. glumae was directly added to molten KB agar to a final concentration of OD₆₀₀=0.001 (1×10⁵ CFU/ml), and poured into sterile Petri plates. After plates solidified, five 6 mm diameter sterile filter paper disks were placed on plates. Five microliters of testing bacteria were added to four of the filter disks, and five microliters of sterile water were added to the fifth disk as a negative control. Plates were incubated at 28° C. and pictures were taken after 48 h. Pictures were uploaded into ImageJ (Rasband, 1997) to calculate the diameter of the zone of inhibition, which is visualized as a clearing around the disks containing potentially inhibitory bacteria. The area of the filter disk was subtracted from the area of the zone of inhibition. Each of the disks were used as replicates for a given experiment, and each combination of pathogen-antagonist was tested in two plates. The experiment was repeated three times. Differences in growth inhibition were determined by one-way analysis of variance (ANOVA) followed by Tukey's honestly significant difference (HSD) tests.

Genome Sequencing and Genome Analysis of P. protegens PBL3

P. protegens PBL3 genomic DNA was extracted from a 3 ml culture grown overnight using the GeneJET Genomic DNA Purification kit (ThermoFisher Scientific, Waltham, MA). DNA quality was visualized in a 1% agarose gel and stained using Gel Red® Nucleic Acid Gel Stain (Biotium, CA, USA). Total DNA was quantified using a Qubit 4 fluorometer (Thermo Fisher Scientific, Waltham, MA) and was sequenced using an Illumina tagmentation library approach on a NextSeq 550 instrument with 150-bp paired-end reads (Baym et al., 2015). A total of 6,768,192 pair-end reads and 926,660, 108 bp (˜47× coverage) were obtained, and the genome was assembled using shovill 1.0.9 (github.com/tseemann/shovill) using parameter “—minlen 500—trim” and defaults for remaining analyses. Initial processing consisted of trimming with Trimmomatic v0.39 (Bolger et al., 2014), followed by error correction with Lighter v1.1.2 (Song et al., 2014) and merging overlapping reads using Flash v1.2.11 (Magoč & Salzberg, 2011). This resulted in 1.4 million reads merged and 1.1 million reads unmerged. These files were used to assemble the genome using spades v3.14.0 (Bankevich et al., 2012) and the resulting assembly was polished with Pilon v1.23 (Walker et al., 2014) resulting in 24 contigs. A second scaffolding approach was done using SSPACE v3.0 (Boetzer et al., 2011), resulting in a 7.04 Mbp assembly containing 14 scaffolds. To finalize the assembly, Pseudomonas protegens CHA0 (NC_021237.1) was used to complete the chromosome using AlignGraph (Bao et al., 2014). The assembly was annotated using prokka v1.14.5 (Seemann, 2014). Raw data was submitted to Sequence Read Archive (SRA) (Bioproject PRJNA626017 and Biosample number SAMN14614890). The genome assembly was also submitted to NCBI (Accession CP051673). In order to identify putative biosynthetic gene clusters involved in the production of secondary metabolites that could be responsible for the antagonistic activity, the genome sequence was mined using antiSMASH (Blin et al., 2019).

The sequence information obtained may be found at the following webpages: “Pprot annotation.txt” at: documentcloud.adobe.com/link/track?uri=urn:aaid:scds:US:2c49034f-c7b5-4ddb-b570-4ald0c927f76 [documentcloud.adobe.com]
“Pprot coordinates.txt” at: documentcloud.adobe.com/link/track?uri=urn:aaid:scds:US:dd2eb2a5-bead-433f-93b0-1ald089f7089 [documentcloud.adobe.com]
“Pprot.txt” at: documentcloud.adobe.com/link/track?uri-urn:aaid:scds:US:1d0948d9-9bf6-4253-9dff-2b707269866a [documentcloud.adobe.com].
   Preparation and Evaluation of the Secreted Fraction From P. protegens PBL3

P. protegens PBL3 cultures were grown overnight in 250 ml of LB broth and incubated at 28° C. by shaking. Cultures were centrifuged for 10 min at 6,000 rpm and the supernatant was collected and lyophilized in a Labonco Freezone® 12 freeze dry system (Labconco, MO, USA) for 3 days until a dried powder was obtained. Sterile LB was also lyophilized using the same conditions and used as a negative control. For the experiments, 0.1 or 0.5 g of lyophilized LB or lyophilized secreted fraction from P. protegens PBL3 were resuspended in 1 ml of sterile water to obtain concentrations of 0.1 g/ml or 0.5 g/ml, respectively, depending on the experiment. Reconstituted LB or reconstituted secreted fraction from P. protegens PBL3 were filter-sterilized using a 0.22 μM filter to remove residual bacteria or possible contamination during lyophilization.

Reconstituted LB or reconstituted cell-free secreted fraction from P. protegens PBL3 were independently added to KB broth at 1:1 ratio in a final volume of 3 ml and used to grow a single colony of B. glumae. Cultures of B. glumae were grown in a shaker at 30° C. for 24 h. After 24 h, cultures were serially diluted and plated on KB to enumerate bacterial populations. This experiment was repeated three times with equivalent results.

To generate sub-fractions of the P. fluorescens PBL3 secreted fraction, the reconstituted secreted fraction at a final concentration of 0.1 g/ml was separated by molecular mass ranges using ultrafiltration centrifugal devices equipped with polyethersulfone (PES) membranes of defined molecular mass cutoffs: 30 kDa, 10 kDa, and 3 kDa (ThermoFisher Scientific, Waltham, MA). Sub-fractions higher and lower than each of the molecular mass cut-offs were filter-sterilized and added to KB. KB alone or supplemented with the subfractions was used to grow B. glumae to evaluate bacterial populations. Each of these experiments was repeated three times.

### Pathogenicity Assays

To evaluate the effect of P. protegens PBL3 on disease development caused by B. glumae, overnight cultures of P. protegens PBL3 and B. glumae were prepared in KB media and diluted to an OD₆₀₀=0.125 (˜1×10⁷ CFU/ml). E. coli DH5α prepared in the same way was used as a negative control. Twenty microliters of B. glumae inoculum alone, or in combination with P. protegens PBL3 or E. coli DH5α at 1:1 ratios were injected into the sheath of eight-week-old plants from moderately susceptible cultivar Wells grown in soil. Plants were also injected with water and used as mock controls. Plants were transferred to growth chambers with day/night temperatures of 35° C./28° C., photoperiod of 16 h light/8 h dark and 60-65% relative humidity for eight days. Plants were monitored each day and lesions were measured at 8 days post-inoculation (dpi). The experiment was repeated three times with similar results.

To evaluate the effect of cell-free secreted fraction from P. protegens PBL3 on the pathogenicity of B. glumae, cell-free reconstituted secreted fraction from P. protegens PBL3 at 0.5 g/L was also added to a prepared inoculum of B. glumae at OD₆₀₀=0.125. Reconstituted LB at 0.5 g/L was also added to a prepared inoculum of B. glumae at OD₆₀₀=0.125, and used as control. Twenty microliters of B. glumae mixed with the cell-free reconstituted secreted fraction from P. protegens PBL3, or mixed with reconstituted LB were injected into the sheaths of 10-week-old Nipponbare rice plants in triplicates. At 2 and 7-days dpi, three inches of sheath (1.5 inches above and below injection site) were ground in 2 ml of sterile water and 0.5 g of sand. Samples were serially diluted and plated on modified CCNT media to enumerate B. glumae populations. Plates were incubated at 28° C. and colonies were counted after two days.

We also evaluated the effect of cell-free reconstituted secreted fraction from P. protegens PBL3 on B. glumae infection on seeds. For that purpose, seeds from cultivar Nipponbare were sterilized as described above. Twenty sterile seeds were inoculated with 2 ml of B. glumae at OD₆₀₀=0.001 (1 ×10⁵ CFU/ml) either containing 0.5 g/ml of lyophilized and resuspended LB, or cell-free lyophilized fractions of P. protegens (0.1 g/ml or 0.5 g/ml). Seeds were incubated with inocula by shaking at 100-150 rpm for 30 min. Bacterial inoculum was decanted and the seeds were plated on Murashige and Skoog (MS) axenic media containing 0.01% Myo-inositol and 0.02% pimaricin (0.2 ml/L). Plates were wrapped with surgical tape, and incubated in a 28° C. in the dark for two days. Plates were then transferred to a growth chamber set up at 25° C. and photoperiod of 16 h light/8 h dark for 5 days. Shoot length of rice seedlings was measured at 7 dpi using ImageJ (Rasband, 1997). All these experiments were repeated three times.

### Statistical Analysis

Means and standard deviations were calculated for all treatments within each experiment. Differences between treatments were determined by analysis of variance (ANOVA) using one-way ANOVA or T-test depending on the number of treatments using JMP Pro 14 software (SAS, NC, USA). Analyses were performed at P=0.05 or P=0.001 level and when differences were significant with ANOVA, means were compared using Tukey's honestly significant difference (HSD).

### Results

Identifying Bacterial Strains With Antagonistic Activity Against B. glumae

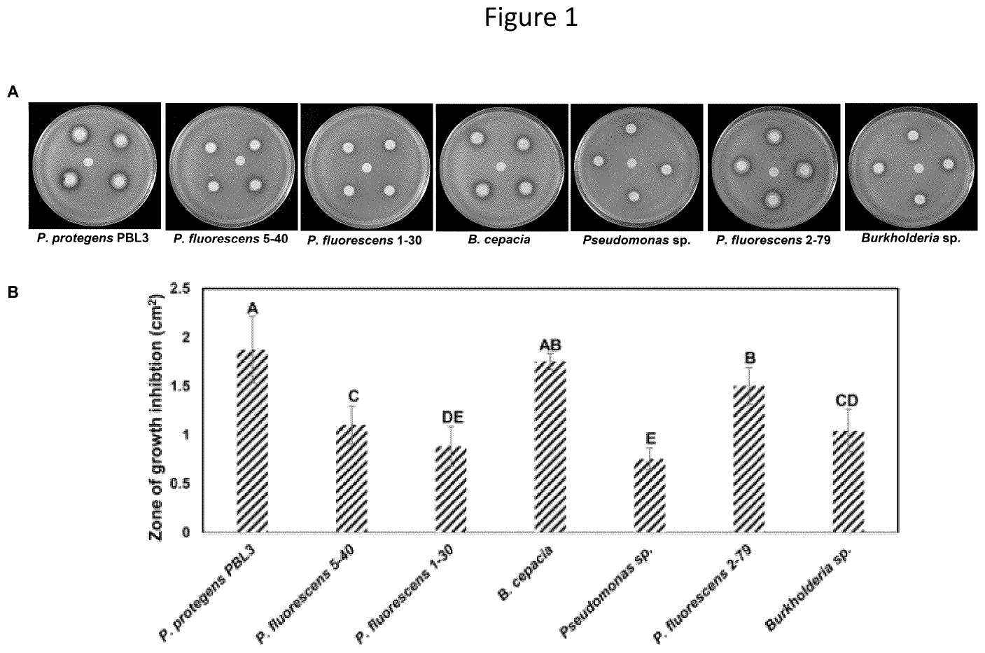

Seven bacterial strains, including P. protegens PBL3, P. fluorescens 5-40, P. fluorescens 1-30, B. cepacia, P. fluorescens sp., P. fluorescens 2-79 and Burkholderia sp., were previously investigated for their potential as biological control agents against soil-borne pathogens (Milus & Rothrock, 1997). Because these strains were already available in our lab, we decided to investigate if they could be used to control bacterial panicle blight of rice. We used a plate inhibition assay wherein B. glumae was grown in the presence of the tested bacteria (FIG. 1). We found that two of the strains: P. protegens PBL3 and B. cepacia significantly inhibited the growth of B. glumae, as shown by a clear area around the disks containing these bacteria (FIG. 1A). Both P. protegens PBL3 and B. cepacia caused the same growth inhibition with an area of 1.75 cm² (FIG. 1B). The other bacteria tested (P. fluorescens 5-40, P. fluorescens 1-30, Pseudomonas sp, P. fluorescens 2-79, and Burkholderia sp) had a reduced effect on growth inhibition that ranged from 0.75 cm² to 1.5 cm² (FIG. 1B). Since other strains of P. protegens have been widely recognized as wide-spectrum biological control organisms, we decided to investigate this bacterium in more detail.

### Bacterial Identification and Genome Sequencing

P. protegens PBL3 was previously named P. fluorescens PFS JA4092. Our initial analysis of the 16S rRNA sequence of P. protegens PBL3 revealed that it had 99% similarity to the 16s rRNA sequence of P. protegens CHA0 (Genbank NR_114749.1), based on the sequences available from the Ribosomal Database Project (RDP) (Cole et al., 2014). To resolve the discrepancy in the original naming, we sequenced the bacterial genome. The assembled scaffolds were submitted to the Ribosomal Multilocus Sequence Typing tool (rMLST; pubmlst.org/rmlst/) (Jolley et al., 2012), and this analysis extracted 54 different regions that supported the ID of the strain as P. protegens (Table 2).

TABLE 2
Regions supporting the identification of P. protegens PBL3

| | | | | Start | End | |
|---|---|---|---|---|---|---|
| Locus | Allele | Length | Contig | position | position | Linked data values |
| BACT000001 | 207 | 1695 | P_protegens_PBL3 | 4678588 | 4680282 | species: Pseudomonas protegens [n = 7] |
| (rpsA) | | | | | | |
| BACT000002 | 177 | 738 | P_protegens_PBL3 | 1332708 | 1333445 | species: Pseudomonas protegens [n = 7] |
| (rpsB) | | | | | | |
| BACT000003 | 144 | 687 | P_protegens_PBL3 | 5853892 | 5854578 | species: Pseudomonas protegens [n = 15]; |
| (rpsC) | | | | | | Pseudomonas sp. [n = 14] |
| BACT000004 | 142 | 621 | P_protegens_PBL3 | 5845765 | 5846385 | species: Pseudomonas protegens [n = 9]; |
| (rpsD) | | | | | | Pseudomonas sp. [n = 7] |
| BACT000005 | 125 | 501 | P_protegens_PBL3 | 5849398 | 5849898 | species: Pseudomonas protegens [n = 7] |
| (rpsE) | | | | | | |
| BACT000006 | 118 | 426 | P_protegens_PBL3 | 667679 | 668104 | species: Pseudomonas protegens [n = 37]; |
| (rpsF) | | | | | | Pseudomonas sp. [n = 18] |
| BACT000007 | 122 | 471 | P_protegens_PBL3 | 5861554 | 5862024 | species: Pseudomonas protegens [n = 29]; |
| (rpsG) | | | | | | Pseudomonas sp. [n = 14] |
| BACT000008 | 106 | 393 | P_protegens_PBL3 | 5850809 | 5851201 | species: Pseudomonas protegens [n = 27]; |
| (rpsH) | | | | | | Pseudomonas sp. [n = 14] |
| BACT000009 | 119 | 393 | P_protegens_PBL3 | 5380880 | 5381272 | species: Pseudomonas protegens [n = 7]; |
| (rpsI) | | | | | | Pseudomonas sp. [n = 7] |
| BACT000010 | 111 | 312 | P_protegens_PBL3 | 5857696 | 5858007 | species: Pseudomonas protegens [n = 35]; |
| (rpsJ) | | | | | | Pseudomonas sp. [n = 14] |
| BACT000011 | 95 | 390 | P_protegens_PBL3 | 5846404 | 5846793 | species: Pseudomonas protegens [n = 29]; |
| (rpsK) | | | | | | Pseudomonas sp. [n = 14] |
| BACT000012 | 119 | 372 | P_protegens_PBL3 | 5862136 | 5862507 | species: Pseudomonas protegens [n = 16] |
| (rpsL) | | | | | | |
| BACT000013 | 105 | 357 | P_protegens_PBL3 | 5846824 | 5847180 | species: Pseudomonas protegens [n = 7] |
| (rpsM) | | | | | | |
| BACT000014 | 94 | 306 | P_protegens_PBL3 | 5851409 | 5851714 | species: Pseudomonas protegens [n = 25]; |
| (rpsN) | | | | | | Pseudomonas sp. [n = 7] |
| BACT000015 | 114 | 270 | P_protegens_PBL3 | 963677 | 963946 | species: Pseudomonas protegens [n = 29]; |
| (rpsO) | | | | | | Pseudomonas sp. [n = 18]; |
| | | | | | | Pseudomonas saponiphila [n = 1] |
| BACT000016 | 117 | 252 | P_protegens_PBL3 | 1234557 | 1234808 | species: Pseudomonas protegens [n = 27] |
| (rpsP) | | | | | | |
| BACT000017 | 105 | 267 | P_protegens_PBL3 | 5853006 | 5853272 | species: Pseudomonas protegens [n = 25]; |
| (rpsQ) | | | | | | Pseudomonas sp. [n = 9] |
| BACT000018 | 102 | 231 | P_protegens_PBL3 | 668133 | 668363 | species: Pseudomonas sp. [n = 120] |
| (rpsR) | | | | | | Pseudomonas protegens [n = 39] |
| | | | | | | Pseudomonas corrugata [n = 7] |
| | | | | | | Pseudomonas koreensis [n = 5] |
| | | | | | | Pseudomonas fluorescens [n = 4] |
| | | | | | | Pseudomonas mandelii [n = 4] |
| | | | | | | Pseudomonas moraviensis [n = 4] |
| | | | | | | Pseudomonas palleroniana [n = 3] |
| | | | | | | Pseudomonas reinekei [n = 3] |
| | | | | | | Pseudomonas vancouverensis [n = 3] |
| | | | | | | Pseudomonas jessenii [n = 2] |
| | | | | | | [Flavobacterium] sp. 29 [n = l] |
| | | | | | | Pseudomonas helmanticensis [n = 1] |
| BACT000019 | 91 | 276 | P_protegens_PBL3 | 5854938 | 5855213 | species: Pseudomonas chlororaphis [n = 56]; |
| (rpsS) | | | | | | Pseudomonas sp. [n = 30]; |
| | | | | | | Pseudomonas protegens [n = 11] |
| BACT000020 | 114 | 279 | P_protegens_PBL3 | 5614781 | 5615059 | species: Pseudomonas protegens [n = 25]; |
| (rpsT) | | | | | | Pseudomonas sp. [n = 14] |
| BACT000021 | 71 | 216 | P_protegens_PBL3 | 5942690 | 5942905 | species: Pseudomonas protegens [n = 30]; |
| (rpsU) | | | | | | Pseudomonas sp. [n = 9] |
| BACT000030 | 163 | 696 | P_protegens_PBL3 | 5872413 | 5873108 | species: Pseudomonas protegens [n = 8] |
| (rplA) | | | | | | |
| BACT000031 | 146 | 825 | P_protegens_PBL3 | 5855230 | 5856054 | species: Pseudomonas protegens [n = 7] |
| (rplB) | | | | | | |
| BACT000032 | 148 | 636 | P_protegens_PBL3 | 5856980 | 5857615 | species: Pseudomonas protegens [n = 7] |
| (rplC) | | | | | | |
| BACT000033 | 130 | 603 | P_protegens_PBL3 | 5856365 | 5856967 | species: Pseudomonas protegens [n = 7] |
| (rplD) | | | | | | |
| BACT000034 | 131 | 540 | P_protegens_PBL3 | 5851728 | 5852267 | species: Pseudomonas protegens [n = 18]; |
| (rplE) | | | | | | Pseudomonas sp. [n = 14] |
| BACT000035 | 148 | 534 | P_protegens_PBL3 | 5850263 | 5850796 | species: Pseudomonas protegens [n = 12]; |
| (rplF) | | | | | | Pseudomonas sp. [n = 9] |
| BACT000036 | 140 | 366 | P_protegens_PBL3 | 5871268 | 5871633 | species: Pseudomonas protegens [n = 29]; |
| (rplL) | | | | | | Pseudomonas sp [n = 9] |
| BACT000038 | 176 | 447 | P_protegens_PBL3 | 669321 | 669767 | species: Pseudomonas protegens [n = 28]; |
| (rplI) | | | | | | Pseudomonas sp. [n = 16]; Pseudomonas |
| | | | | | | saponiphila [n = 1] |
| BACT000039 | 166 | 501 | P_protegens_PBL3 | 5871714 | 5872214 | species: Pseudomonas protegens [n = 28]; |
| (rplJ) | | | | | | Pseudomonas sp. [n = 14] |
| BACT000040 | 150 | 432 | P_protegens_PBL3 | 5873108 | 5873539 | species: Pseudomonas protegens [n = 14]; |
| (rplK) | | | | | | Pseudomonas sp [n = 7] |
| BACT000042 | 131 | 429 | P_protegens_PBL3 | 5381287 | 5381715 | species: Pseudomonas protegens [n = 27]; |
| (rplM) | | | | | | Pseudomonas sp. [n = 7] |
| BACT000043 | 115 | 369 | P_protegens_PBL3 | 5852614 | 5852982 | species: Pseudomonas protegens [n = 26]; |
| (rplN) | | | | | | Pseudomonas sp. [n = 14] |
| BACT000044 | 120 | 435 | P_protegens_PBL3 | 5848781 | 5849215 | species: Pseudomonas protegen [n = 20]; |
| (rplO) | | | | | | Pseudomonas sp [n = 9] |
| BACT000045 | 96 | 414 | P_protegens_PBL3 | 5853466 | 5853879 | species: Pseudomonas protegens [n = 7] |
| (rplP) | | | | | | |
| BACT000046 | 111 | 387 | P_protegens_PBL3 | 5844312 | 5844698 | species: Pseudomonas protegens [n = 17]; |
| (rplQ) | | | | | | Pseudomonas sp. [n = 7] |
| BACT000047 | 124 | 351 | P_protegens_PBL3 | 5849902 | 5850252 | species: Pseudomonas protegens [n = 28]; |
| (rplR) | | | | | | Pseudomonas sp. [n = 15] |
| BACT000048 | 130 | 351 | P_protegens_PBL3 | 1236151 | 1236501 | species: Pseudomonas protegens [n = 27]; |
| (rplS) | | | | | | Pseudomonas sp. [n = 14] |
| BACT000049 | 125 | 357 | P_protegens_PBL3 | 2264001 | 2264357 | species: Pseudomonas protegens [n = 29]; |
| (rplT) | | | | | | Pseudomonas sp. [n = 14] |
| BACT000050 | 112 | 312 | P_protegens_PBL3 | 5618674 | 5618985 | species: Pseudomonas protegens [n = 28]; |
| (rplU) | | | | | | Pseudomonas sp. [n = 17] |
| BACT000051 | 212 | 333 | P_protegens_PBL3 | 5854592 | 5854924 | species: Pseudomonas chlororaphis [n = 58]; |
| (rplV) | | | | | | Pseudomonas protegens [n = 38]; |
| | | | | | | Pseudomonas sp. [n = 26]; |
| | | | | | | Pseudomonas lutea [n = 1]; |
| | | | | | | Pseudomonas saponiphila [n = 1] |
| BACT000052 | 203 | 300 | P_protegens_PBL3 | 5856069 | 5856368 | species: Pseudomonas chlororaphis [n = 17]; |
| (rplW) | | | | | | Pseudomonas protegens [n = 13]; |
| | | | | | | Pseudomonas sp. [n = 5] |
| BACT000053 | 218 | 315 | P_protegens_PBL3 | 5852288 | 5852602 | species: Pseudomonas protegens [n = 28]; |
| (rplX) | | | | | | Pseudomonas sp. [n = 14] |
| BACT000056 | 266 | 258 | P_protegens_PBL3 | 5618381 | 5618638 | species: Pseudomonas protegens [n = 10]; |
| (rpmA) | | | | | | Pseudomonas sp. [n = 7] |
| BACT000057 | 137 | 237 | P_protegens_PBL3 | 6364793 | 6365029 | species: Pseudomonas protegens [n = 31]; |
| (rpmB) | | | | | | Pseudomonas sp. [n = 15] |
| BACT000058 | 160 | 192 | P_protegens_PBL3 | 5853275 | 5853466 | species: Pseudomonas sp. [n = 52]; |
| (rpmC) | | | | | | Pseudomonas protegens [n = 39]; |
| | | | | | | Pseudomonas jessenii [n = 2]; |
| | | | | | | Pseudomonas laurylsulfatiphila [n = 1]; |
| | | | | | | Pseudomonas mandelii [n = 1]; |
| | | | | | | Pseudomonas umsongensis [n = 1] |
| BACT000059 | 135 | 177 | P_protegens_PBL3 | 5849219 | 5849395 | species: Pseudomonas protegens [n = 7] |
| (rpmD) | | | | | | |
| BACT000060 | 193 | 270 | P_protegens_PBL3 | 5196045 | 5196314 | species: Pseudomonas protegens [n = 25] |
| (rpmE) | | | | | | |
| BACT000061 | 156 | 183 | P_protegens_PBL3 | 1977194 | 1977376 | species: Pseudomonas protegens [n = 8]; |
| (rpmF) | | | | | | Pseudomonas sp. [n = 7] |
| BACT000063 | 234 | 135 | P_protegens_PBL3 | 6566162 | 6566296 | species: Pseudomonas sp. [n = 158] |
| (rpmH) | | | | | | Pseudomonas chlororaphis [n = 69] |
| | | | | | | Pseudomonas protegens [n = 39] |
| | | | | | | Pseudomonas brassicacearum [n = 17] |
| | | | | | | Pseudomonas corrugata [n = 7] |
| | | | | | | Pseudomonas mandelii [n = 6] |
| | | | | | | Pseudomonas mediterranea [n = 6] |
| | | | | | | Pseudomonas kilonensis [n = 5] |
| | | | | | | Pseudomonas fluorescens [n = 4] |
| | | | | | | Pseudomonas lini [n = 4] |
| | | | | | | Pseudomonas thivervalensis [n = 4] |
| | | | | | | Pseudomonas reinekei [n = 3] |
| | | | | | | Pseudomonas vancouverensis [n = 3] |
| | | | | | | Pseudomonas jessenii [n = 2] |
| | | | | | | Pseudomonas arsenicoxydans [n = l] |
| | | | | | | Pseudomonas saponiphila [n = l] |
| | | | | | | Pseudomonas umsongensi [n = 1] |
| BACT000064 | 150 | 195 | P_protegens_PBL3 | 2263776 | 2263970 | species: Pseudomonas sp. [n = 114]; |
| (rpmI) | | | | | | Pseudomonas chlororaphis [n = 68]; |
| | | | | | | Pseudomonas protegens [n = 39]; |
| | | | | | | Pseudomonas fluorescens [n = 6]; |
| | | | | | | Pseudomonas brassicacearum [n = 2]; |
| | | | | | | Pseudomonas kribbensis [n = 2]; |
| | | | | | | Pseudomonas asplenii [n = 1]; |
| | | | | | | Pseudomonas koreensis [n = 1]; |
| | | | | | | Pseudomonas saponiphil [n = 1] |
| BACT000065 | 19076 | 150 | P_protegens_PBL3 | 4092111 | 4092260 | species: Pseudomonas protegens [n = 26]; |
| (rpmJ) | | | | | | Pseudomonas sp. [n = 14] |
| BACT000065 | 137 | 117 | P_protegens_PBL3 | 5847311 | 5847427 | species: Pseudomonas sp. [n = 451] |
| (rpmJ) | | | | | | Pseudomonas chlororaphis [n = 71] |
| | | | | | | Pseudomonas fluorescens [n = 63] |
| | | | | | | Pseudomonas protegens [n = 38] |
| | | | | | | Pseudomonas synxantha [n = 11] |
| | | | | | | Pseudomonas koreensis [n = 9] |
| | | | | | | Pseudomonas lurida [n = 9] |
| | | | | | | Pseudomonas veronii [n = 9] |
| | | | | | | Pseudomonas lactis [n = 8] |
| | | | | | | Pseudomonas corrugata [n = 7] |
| | | | | | | Pseudomonas simiae [n = 7] |
| | | | | | | Pseudomonas azotoformans [n = 6] |
| | | | | | | Pseudomonas mediterranea [n = 6] |
| | | | | | | Pseudomonas moraviensis [n = 6] |
| | | | | | | Pseudomonas prosekii [n = 6] |
| | | | | | | Pseudomonas tolaasii [n = 6] |
| | | | | | | Pseudomonas lini [n = 5] |
| | | | | | | Pseudomonas marginalis [n = 5] |
| | | | | | | Pseudomonas palleroniana [n = 5] |
| | | | | | | Pseudomonas rhodesiae [n = 5] |
| | | | | | | Pseudomonas extremorientalis [n = 4] |
| | | | | | | Pseudomonas gessardii [n = 4] |
| | | | | | | Pseudomonas mucidolens [n = 4] |
| | | | | | | Pseudomonas poae [n = 4] |
| | | | | | | Pseudomonas agarici [n = 3] |
| | | | | | | Pseudomonas cedrina [n = 3] |
| | | | | | | Pseudomonas libanensis [n = 3] |
| | | | | | | Pseudomonas mandelii [n = 3] |
| | | | | | | Pseudomonas orientalis [n = 3] |
| | | | | | | Pseudomonas proteolytica [n = 3] |
| | | | | | | Pseudomonas reinekei [n = 3] |
| | | | | | | Pseudomonas salomonii [n = 3] |
| | | | | | | Pseudomonas trivialis [n = 3] |
| | | | | | | Pseudomonas vancouverensis [n = 3] |
| | | | | | | Pseudomonas antarctica [n = 2] |
| | | | | | | Pseudomonas arsenicoxydans [n = 2] |
| | | | | | | Pseudomonas baetica [n = 2] |
| | | | | | | Pseudomonas canadensis [n = 2] |
| | | | | | | Pseudomonas costantinii [n = 2] |
| | | | | | | Pseudomonas grimontii [n = 2] |
| | | | | | | Pseudomonas jessemi [n = 2] |
| | | | | | | Pseudomonas kribbensis [n = 2] |
| | | | | | | Pseudomonas migulae [n = 2] |
| | | | | | | Pseudomonas moorei [n = 2] |
| | | | | | | Pseudomonas yamanorum [n = 2] |
| | | | | | | [Flavobacterium] sp. 29 [n = l] |
| | | | | | | Pseudomonas brenneri [n = l] |
| | | | | | | Pseudomonas extremaustralis [n = l] |
| | | | | | | Pseudomonas granadensis [n = l] |
| | | | | | | Pseudomonas haemolytica [n = l] |
| | | | | | | Pseudomonas helmanticensis [n = l] |
| | | | | | | Pseudomonas kairouanensis [n = l] |
| | | | | | | Pseudomonas lamylsulfatiphila [n = l] |
| | | | | | | Pseudomonas laurylsulfativorans [n = l] |
| | | | | | | Pseudomonas mohnii [n = l] |
| | | | | | | Pseudomonas nabeulensis [n = l] |
| | | | | | | Pseudomonas panacis [n = l] |
| | | | | | | Pseudomonas paralactis [n = l] |
| | | | | | | Pseudomonas saponiphila [n = l] |
| | | | | | | Pseudomonas silesiensis [n = l] |
| | | | | | | Pseudomonas umsongensis [n = 1] |

Pseudomonas protegens PBL3 Reduces Disease Symptoms Caused by B. glumae

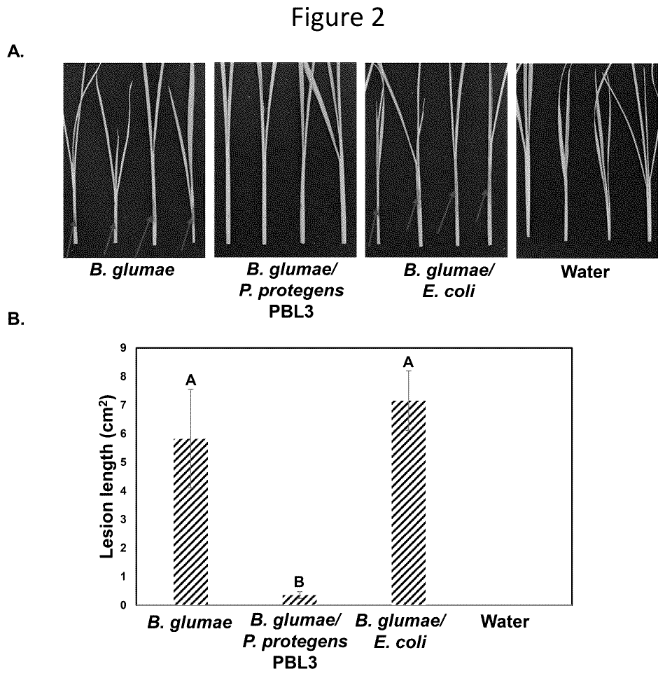

Our initial observations that P. protegens PBL3 inhibited the growth of B. glumae prompted us to investigate its effect controlling bacterial panicle blight of rice under controlled conditions. For that purpose, we co-inoculated B. glumae with P. protegens PBL3 into the susceptible rice cultivar Wells to evaluate symptoms and compare them with those in plants inoculated with B. glumae alone. Plants inoculated with B. glumae alone showed disease symptoms in the stem characterized by brown lesions surrounding the area of inoculation. However, plants that were co-inoculated with B. glumae and P. protegens PBL3 had very small 10 lesions (0.36 cm²), almost indistinguishable from plants treated with water (0 cm²). The reduction in symptoms upon co-inoculation with P. protegens PBL3 was specific to this bacterium, as no reduction of symptoms was observed when B. glumae was co-inoculated with Escherichia coli DH5α (FIG. 2A). The lesion sizes for plants inoculated with B. glumae alone or co-inoculated with E. coli DH5α were 7.16 cm and 5.83 cm, respectively, but did not appear to be different based on statistical analyses. In contrast, co-inoculation of B. glumae and P. protegens PBL3 resulted in a small lesion sizes with an average lesion size of 0.36 cm that were not statistically different from plants treated with water (FIG. 2B). These results suggest that P. protegens PBL3 directly, or indirectly interferes with the pathogenesis of B. glumae on rice. The genome of P. protegens PBL3 contains gene clusters associated with the synthesis of secondary metabolites

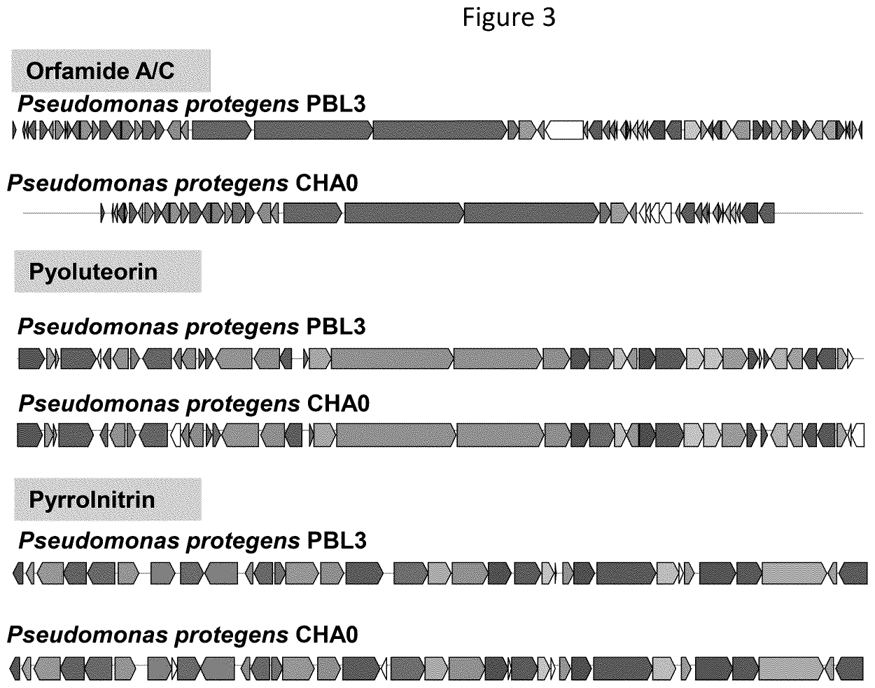

To gain mechanistic insight into the antimicrobial activity of P. protegens PBL3, we initially investigated the genomic capacity of P. protegens PBL3 to synthesize secondary metabolites known to be important in other biological control agents. For that purpose, we searched the genome of P. protegens PBL3 looking for biosynthetic gene clusters involved in the production of secondary metabolites. The analysis using the antiSMASH database (Blin et al., 2019) identified 14 putative gene clusters related to biosynthesis of secondary metabolites (Table 3). The identified genes clusters included biosynthetic genes encoding orfamide A and C (Gross et al., 2007), pyoluteorin, and pyrrolnitrin, which are known secondary metabolites responsible for antifungal activities in the closely related reference strain P. protegens CHA0 (FIG. 3). The gene clusters necessary for the synthesis of orfamide A and C, pyoluteorin, and pyrrolnitrin in P. protegens PBL3 exhibited remarkably similar gene organization and gene content to the respective gene clusters in P. protegens CHA0. In the orfamide A and C gene cluster, 88% of the genes are shared between P. protegens PBL3 and P. protegens CHA0. In the Pyoluteorin gene cluster, there are two additional genes in P. protegens PBL3 that are not present in P. protegens CHA0, and one gene present in P. protegens CHA0 is not present in P. protegens PBL3. Other than those differences, the gene organization of both clusters is almost identical and 92% of the genes are conserved between P. protegens PBL3 and P. protegens CHA0. Regarding the Pyrrolnitrin gene cluster, we found that P. protegens PBL3 has an additional gene that is not present in P. protegens CHA0, while in turn, P. protegens CHA0 has two additional genes that were not found in P. protegens PBL3. Other than that, 88% of the genes are conserved between both strains.

TABLE 3
Putative biosynthetic gene clusters identified in P. protegens PBL3

| PBL3 Genomic | | Most similar known | | |
|---|---|---|---|---|
| region | | cluster | Type | Similarity |
| 4,050,249 | 4,091,334 | Pyrrolnitrin | Pyrrole | 100% |
| 3,101,348 | 3,154,097 | Pyoluteorin | Polyketide | 100% |
| 6,522,690 | 6,563,739 | 2,4-diacetylphloroglucinol | Polyketide | 100% |
| 2,371,841 | 2,465,198 | Orfamide A/orfamide C | NRPS^{a}: Cyclic | 94% |
| | | | depsipeptide | |
| 508,965 | 552,582 | APE Vf | Arylpolyene | 40% |
| | | | (APE) | |
| | | | (Pigment) | |
| 4,628,974 | 4,698,141 | Pyoverdin | NRPS | 33% |
| 3,914,956 | 3,960,628 | Thiazostatin/watasemycin | NRPS | 26% |
| | | A/watasemycin B/2- | | |
| | | hydroxyphenylthiazoline | | |
| | | enantiopyochelin/ | | |
| | | isopyochelin | | |
| 4,769,517 | 4,820,431 | Pyoverdin | NRPS | 15% |
| 4,460,042 | 4,483,256 | Fengycin | NRPS: | 13% |
| | | | Bectalactone | |
| 5,278,248 | 5,329,277 | Lipopeptide 8D1-1/ | NRPS | 6% |
| | | lipopeptide 8D1-2 | | |
| 1,471,397 | 1,480,243 | Bacteriocin | Bacteriocin | |
| 6,794,167 | 6,805,012 | Bacteriocin | Bacteriocin | |
| 1,570,952 | 1,591,701 | CDPS | Cyclodipeptides | |
| 4,967,589 | 4,982,421 | NAGGN | N- | |
| | | | acetylglutaminylglutamine | |
| | | | amide | |

^{a}Non-ribosomal peptide synthetase cluster

The Secreted Fraction From P. protegens PBL3 Reduces the Growth of B. glumae

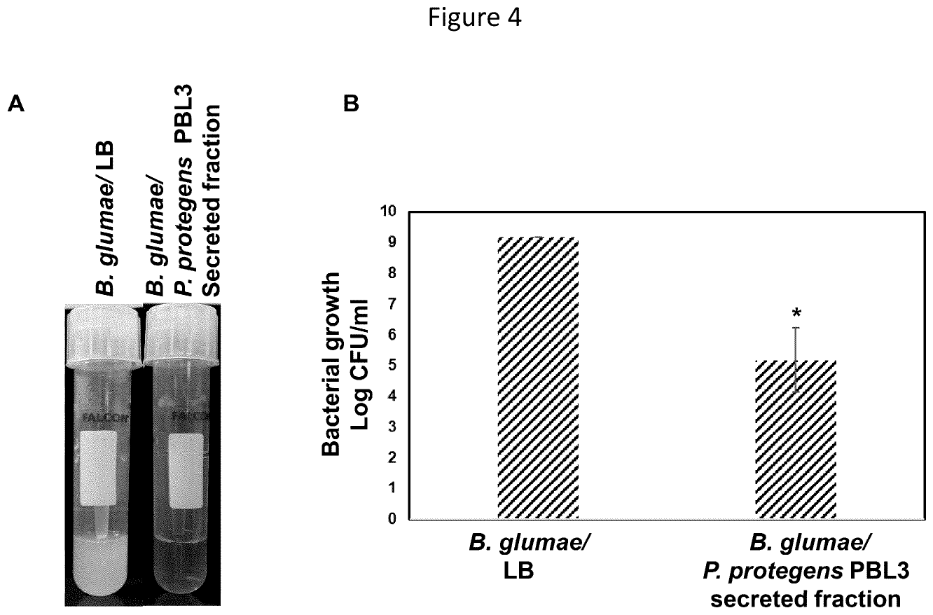

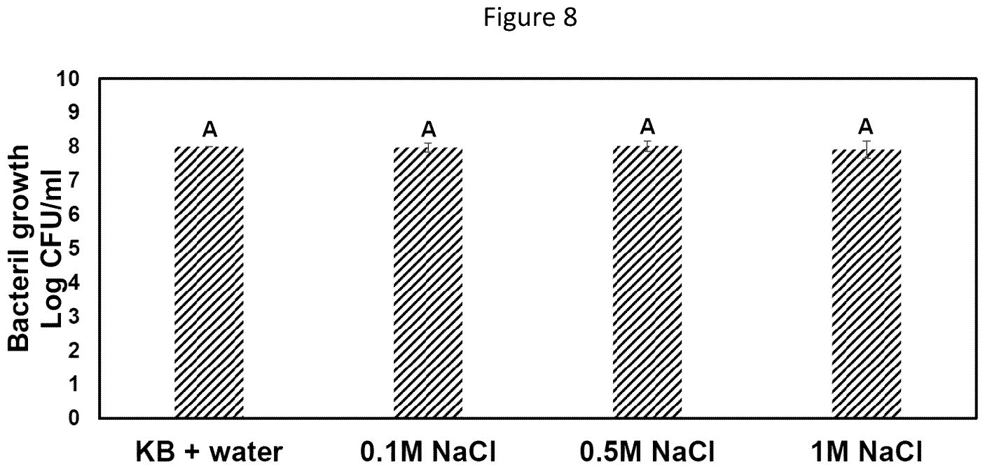

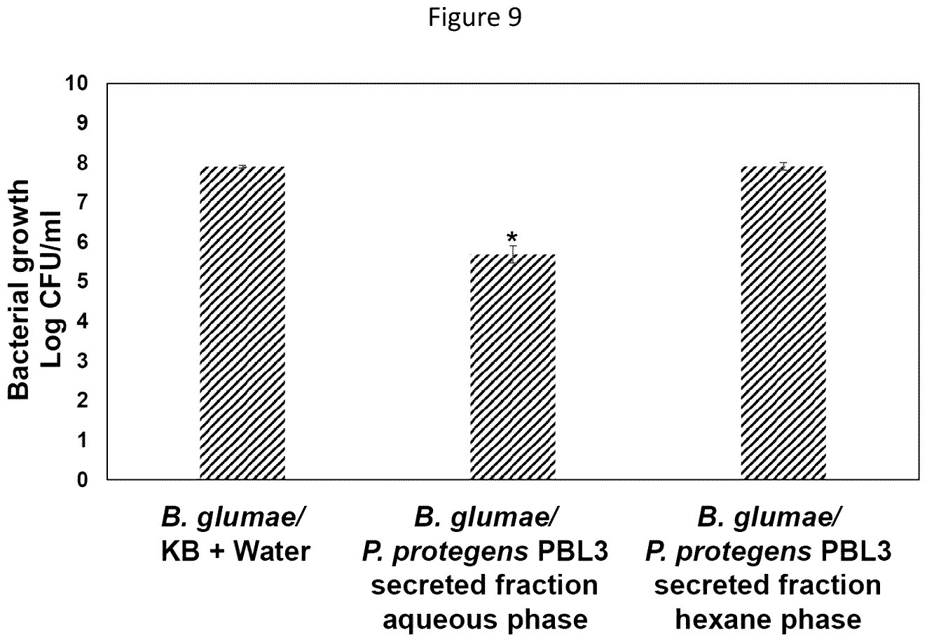

We hypothesized that the observed inhibitory effect of P. protegens PBL3 on the growth of B. glumae involves secreted compounds. To test this hypothesis, we obtained and lyophilized the culture supernatant from P. protegens PBL3, which contains bacterial secretions. The lyophilized secreted fraction was reconstituted and filter-sterilized to completely remove residual bacteria and ensure that the results obtained were exclusively associated with the secreted fraction and not with residual P. protegens PBL3. To test the effect of the lyophilized and resuspended secreted fraction of P. protegens PBL3 on the growth of B. glumae, we added this lyophilized and resuspended secreted fraction of P. protegens PBL3 to the culture media (KB broth), and used this amended KB to grow a single colony of B. glumae. Lyophilized and resuspended LB was also used to amend KB and used as control. After overnight incubation, we observed that the growth of B. glumae on KB amended with lyophilized and resuspended LB was dense, while its growth in KB amended with the lyophilized and resuspended secreted fraction from P. protegens was reduced, as shown by decreased turbidity (FIG. 4A). We further quantified the growth of B. glumae by assessing numbers of bacteria and found that the number of B. glumae in KB amended with lyophilized and resuspended LB reached 10⁹ CFU/ml, while the number of B. glumae in KB amended with the lyophilized secreted fraction from P. protegens PBL3 reached 10⁵ CFU/ml (FIG. 4B). Because growth inhibition was only observed in KB amended with the lyophilized and resuspended secreted fraction derived from P. protegens PBL3 and not in KB amended with lyophilized and resuspended LB, this growth inhibition was directly related to molecules present in the secreted fraction of P. protegens PBL3 and not an indirect effect of the increase in NaCl that resulted from the lyophilization. Independent experiments confirmed that B. glumae can grow in KB supplemented with NaCl in concentrations up to 1 M (FIG. 8). We focused on the water-soluble secreted fraction of P. protegens PBL3, because hexane-extracted fraction was devoid of antimicrobial activity (FIG. 9).

The Secreted Fraction From P. protegens PBL3 Reduces the Disease Symptoms Caused by B. glumae

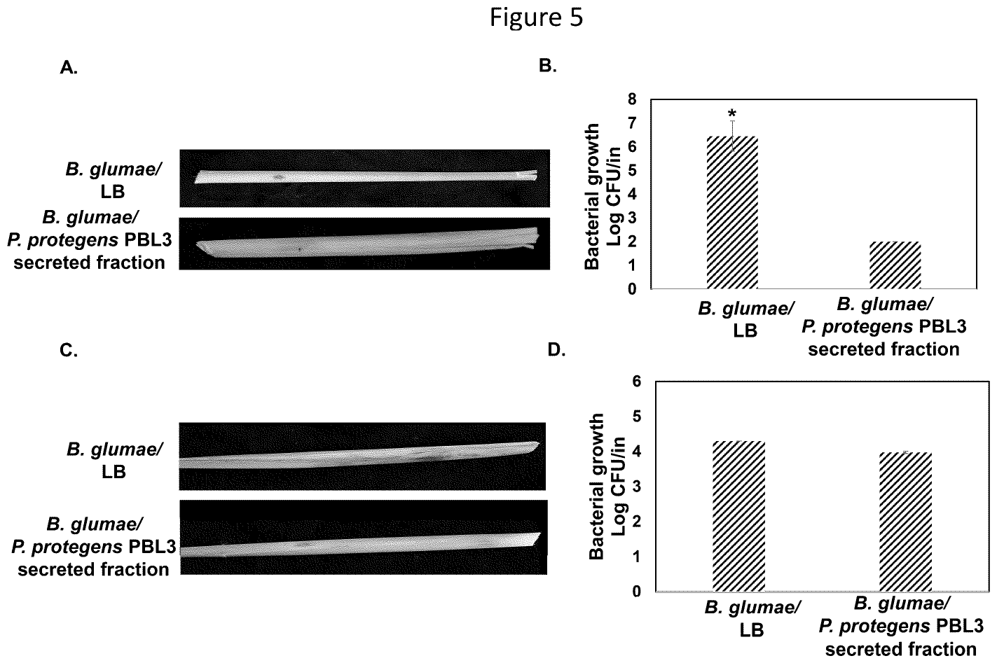

To evaluate the effect of the secreted fraction from P. protegens PBL3 on B. glumae and its ability to cause disease in rice, lyophilized and resuspended cell-free secreted fractions from P. protegens PBL3, or lyophilized and resuspended LB were added to B. glumae inoculum and injected into rice plants. Disease development and bacterial populations were investigated at 2 and 7 days post-injection (dpi). At 2 dpi, B. glumae mixed with the lyophilized and resuspended LB or with the lyophilized and resuspended secreted fraction of P. protegens PBL3 did not produce disease symptoms (FIG. 5A). Interestingly, at 2 dpi, we observed that plants inoculated with B. glumae mixed with the lyophilized and resuspended LB showed 5-log higher bacterial populations than plants inoculated with B. glumae containing the lyophilized and resuspended cell-free secreted fraction from P. protegens PBL3 (FIG. 5B). This result demonstrates that the secreted fraction from P. protegens PBL3 contains antagonistic activity against B. glumae. At 7 dpi, plants inoculated with B. glumae mixed with the lyophilized and resuspended LB showed large lesions (6 cm) that extended beyond the inoculation site throughout the sheath, while plants inoculated with B. glumae containing the lyophilized and resuspended cell-free secreted fraction from P. protegens PBL3 had a very small lesion (0.48 cm) around the site of inoculation (FIG. 5C). Intriguingly, at 7 dpi, there was no difference in bacterial populations between plants inoculated with B. glumae mixed with the lyophilized and resuspended LB or B. glumae containing the lyophilized and resuspended cell-free secreted fraction from P. protegens PLB3 (FIG. 5D). Altogether, these results showed that the secreted fraction from P. protegens PBL3 can inhibit bacterial growth at 2 dpi and reduce the onset of disease symptoms at 7 dpi.

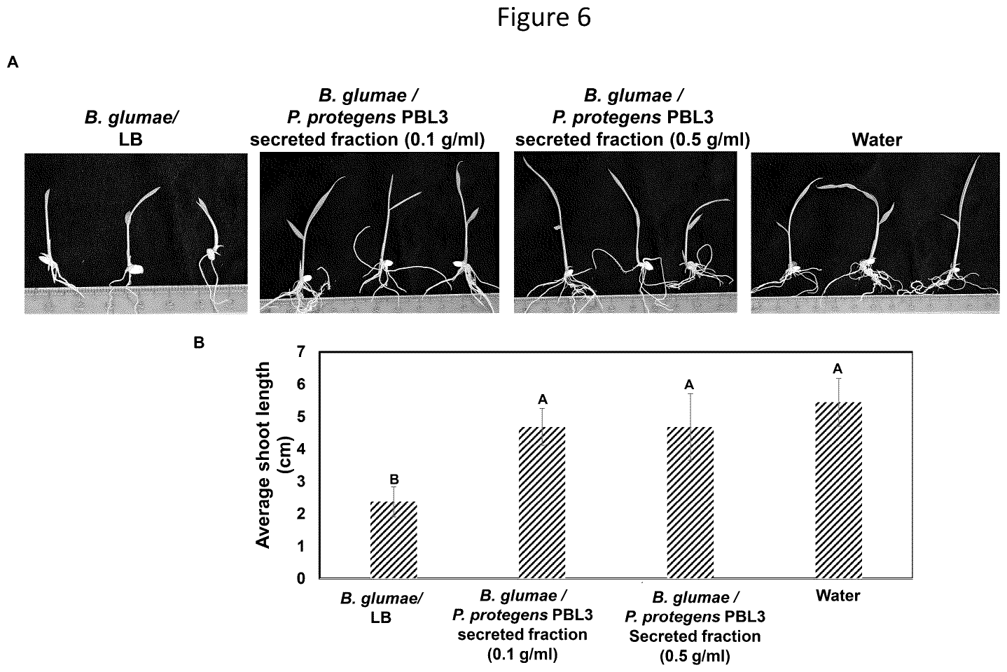

To evaluate other effects of the secreted fraction of P. protegens PLB3 on B. glumae pathogenesis, Nipponbare seeds were inoculated with B. glumae mixed with lyophilized and resuspended LB or with the lyophilized and resuspended cell-free secreted fraction from P. protegens PBL3. As shown in FIG. 6, B. glumae interferes with either seed germination or shoot growth as seeds inoculated with B. glumae mixed with lyophilized and resuspended LB showed reduced shoot length in comparison with water-treated seeds (FIGS. 6A and B). In contrast, seeds inoculated with B. glumae containing the lyophilized and resuspended cell-free secreted fraction from P. protegens PBL3 at two different concentrations developed normally (FIG. 6A), and their shoot length of approximately 2 cm was not significantly different from water-treated seeds (FIG. 6B).

The Antimicrobial Activities of P. fluorescens PBL3 Against B. glumae are Related to Molecules of Different Molecular Mass

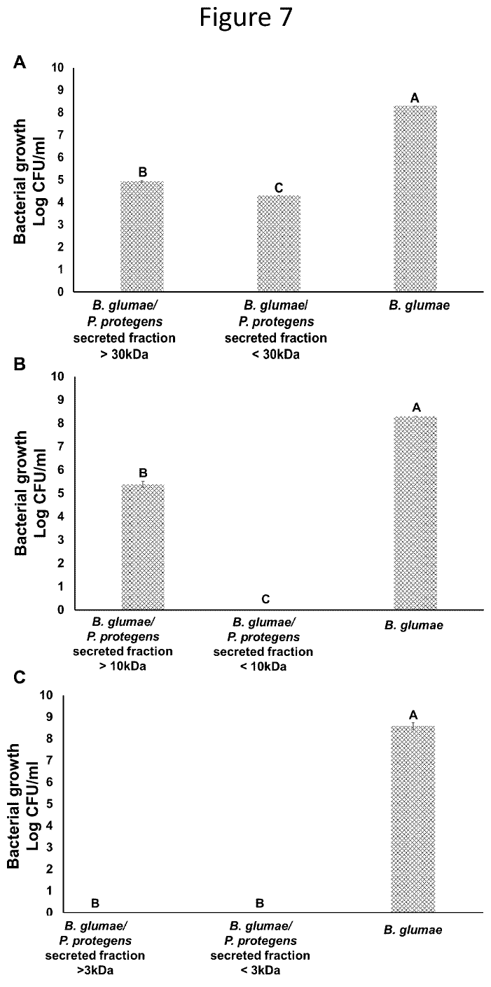

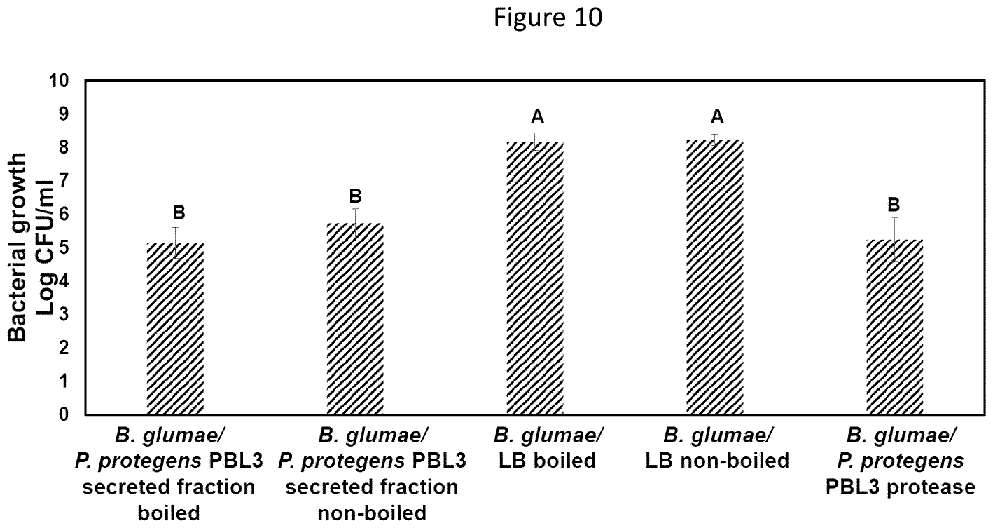

To start dissecting the antimicrobial activity in the P. protegens PBL3 secreted fraction, we decided to sub-fractionate it into molecular ranges by using an ultrafiltration centrifugal concentrator of 30 kDa cut-off that separated the P. protegens secreted fraction into sub-fractions higher and lower than 30 kDa. We used those fractions to amend KB broth, and that amended KB broth was then used to grow B. glumae. B. glumae without any secreted fraction grew to 10⁹ CFU/ml. However, sub-fractions higher than 30 kDa and lower than 30 kDa significantly reduced the growth of B. glumae to 10⁵ and 10⁴ CFU/ml, respectively (FIG. 7A). This result led us to hypothesize that the activity associated with large molecular weight fractions could be attributed to proteins. However, boiling and protease treatment did not reduce the antimicrobial activity of the P. protegens PBL3 secreted fraction (FIG. 10), demonstrating that the P. protegens PBL3 antimicrobial activity in the secreted fraction is not associated with proteins.

Similar assays using ultrafiltration centrifugal concentrators with molecular weight cut-off of 10 kDa, showed that the sub-fraction higher than 10 kDa reduced bacterial growth by ˜3 logs (FIG. 7B). Interestingly, the sub-fraction lower than 10 kDa completely inhibited the growth of B. glumae (FIG. 7B). Using the ultrafiltration centrifugal concentrators of 3 kDa showed that both sub-fractions, higher than 3 kDa and lower than 3 kDa completely inhibited the growth of B. glumae (FIG. 7C). The growth inhibition by these smaller molecular size fractions indicates that the activity could be attributed to peptides or secondary metabolites. More work is needed to distinguish between these and other possibilities. Nevertheless, the finding that the antimicrobial activity is found in sub-fractions of different molecular ranges indicates that the overall antimicrobial activity is due to several molecules of different chemical composition.

### Discussion

Bacterial plant diseases are difficult to control and usually require integrated management approaches incorporating cultural practices, plant host resistance, and chemical or biological control (Sundin et al., 2016). Chemical control has mostly relied on copper-containing products or antibiotics that, unfortunately, lose effectiveness over time because bacteria develop resistance, making their use unsustainable (Sundin & Bender, 1993, Manulis et al., 2003, Rodriguez et al., 2006, Barranquero et al., 2013). In the case of bacterial panicle blight of rice, an integrated management approach to control the disease is not feasible because completely resistant cultivars are not available and chemical or biological control methods have proven to be ineffective (Maeda et al., 2004, Shrestha et al., 2016). Considering that rice is a major food staple for the vast majority of people, this disease can have detrimental consequences globally. Thus, identifying sustainable methods to control it is an urgent need.

In this work, we identified one strain of P. protegens, strain PBL3, that inhibited the growth of B. glumae in vitro, and reduced or eliminated disease symptoms upon co-inoculation with the bacterial pathogen B. glumae under controlled conditions. We further showed that the secreted fraction of P. protegens PBL3 is biologically active against B. glumae. The antagonistic effect of P. protegens PBL3, or molecules derived from it, on B. glumae has not been reported before. This discovery is important, because although several Pseudomonas sp. have been commonly recognized as biological control agents (Haas & Defago, 2005), most studies have focused on their effect on fungal or oomycete pathogens and not in bacterial pathogens.

The biological control strategies that Pseudomonas sp. utilizes have been extensively characterized (Haas & Defago, 2005). These strategies mostly rely on competition for nutrients and antibiosis. It is well-known that Pseudomonas sp. competes with pathogens for iron through the production of the siderophore pseudobactin that chelates iron and by doing so, prevents pathogens from acquiring this essential element (Haas & Defago, 2005). Studies have demonstrated that this strategy has enabled Pseudomonas sp. to control diseases caused by the fungal pathogens Fusarium oxysporum and Gaeumannomyces graminis var. tritici (Kloepper et al., 1980, Lemanceau et al., 1992), and the bacterial pathogen Xanthomonas oryzae pv. oryzae (Yasmin et al., 2016). Several Pseudomonas sp. are also known for producing several antibiotics, defined as “low molecular weight secondary metabolites with antimicrobial activities” (Thomashow et al., 1997), including: phloroglucinols, phenazines, pyoluteorin, pyrrolnitrin, pyocyanine, hydrogen cyanide (HCN), and cyclic lipopeptides such as orfamides (Haas & Defago, 2005, Weller, 2007). Each one of these antibiotics has been associated with the control of specific plant diseases caused by fungal or oomycete pathogens especially soil-borne pathogens (Haas & Defago, 2005). For example, P. fluorescens produces 2,4-diacetylphloroglucinol that was demonstrated to be effective for suppressing the fungal pathogens Thielaviopsis basicola and Gaeumannomyces graminis var. tritici (Keel et al., 1992). Phenazines from P. fluorescens have activity against Gaeumannomyces graminis var. tritici, Fusarium oxysporum and Rhizoctonia solani (Thomashow and Weller, 1988, Chin et al., 2000, Mazurier et al., 2009, D'Aes et al., 2011). Pyoluteorin had activity against Pythium ultimum (Howell & Stipanovic, 1980, Maurhofer et al., 1994), while Pyrrolnitrin was effective against Rhizoctonia solani (Howell & Stipanovic, 1979). Moreover, analysis of wild-type and mutant strains of P. fluorescens under controlled conditions revealed the importance of HCN and cyclic lipopeptides in controlling Thielaviopsis basicola and Rhizoctonia solani, respectively (Voisard et al., 1989, D'Aes et al., 2011, Olorunleke et al., 2015).

Our initial experiments showing that co-inoculation of P. protegens PBL3 with B. glumae reduces disease symptoms suggests that P. protegens may be useful for controlling bacterial panicle blight of rice. However, understanding the mechanisms behind this observation is critical to properly design a biological control strategy that would be effective under field conditions. Our co-inoculation experiments did not allow us to distinguish between competition or antibiosis. However, analysis of the P. protegens PBL3 genome revealed that it contains a repertoire of gene clusters encoding secondary metabolites, similar to those present in other fluorescent Pseudomonas sp. with demonstrated antibiosis against plant pathogens. This information led us to hypothesize that our strain of P. protegens PBL3 use antibiosis to control B. glumae. Because secondary metabolites are secreted molecules, we tested that hypothesis by preparing a cell-free secreted fraction from P. protegens PBL3 and testing its activity in vitro and in planta. Our results showed that the cell-free secreted fraction from P. protegens PBL3 and sub-fractions of defined molecular mass significantly reduced the growth of B. glumae in vitro. Moreover, the addition of the cell-free secreted fraction to the B. glumae inoculum revealed important effects on the pathogenesis of B. glumae including: 1) reduction of bacterial numbers in planta at early stages of pathogenesis, 2) reduction of disease symptoms at later stages of pathogenesis and, 3) protection of seed germination. Our current data favors a model in which the secreted fraction of P. protegens PBL3 have a bacteriostatic effect inhibiting the growth of B. glumae but not killing it. Thus, at early stages of pathogenesis, secretions of P. protegens PBL3 interfere with bacterial growth as reflected in the difference in cell numbers between plants inoculated with B. glumae containing resuspended LB and plants inoculated with B. glumae containing the resuspended secreted fraction of P. protegens PBL3. However, those differences in bacterial populations had no effect in the onset of the disease. However, at later stages of pathogenesis, those initial differences in bacterial populations have an effect in the development of symptoms although indirectly, likely associated with the production of toxoflavin. Because the production of toxoflavin by B. glumae is regulated by quorum sensing (Kim et al., 2004), the initially larger populations of B. glumae in plants that were inoculated with B. glumae containing resuspended LB causes these plants to reach the population thresholds required for the production of toxoflavin earlier than plants that were inoculated with B. glumae containing the resuspended secreted fraction of P. protegens PBL3. It is also possible that molecules produced by P. protegens trigger plant defense responses at the expense of the pathogen, another facet of Pseudomonas sp, triggering induced systemic resistance (ISR) (Pieterse et al., 2014). More work is needed to distinguish between these two possibilities.

This work does not present direct evidence that P. protegens PBL3 actually produces the antibiotics that its genomic sequence predicts, and does not pinpoint a particular molecule or molecules with biological activity against B. glumae. Rather, it provides evidence that the secreted fraction of P. protegens PBL3, containing a complex mixture of potentially bioactive molecules, has activity against B. glumae, likely due to the cumulative effect of several molecules with diverse mode of actions. This finding is important, as it will pave the way towards using the cell-free secreted fraction of P. protegens PBL3 to combat bacterial panicle blight of rice instead of using live P. protegens.

The use of living organisms to control plant diseases is challenging because the production of bioactive molecules by such organisms depends on multiple conditions (Jamali et al., 2009, Raaijmakers & Mazzola, 2012), that are not necessarily easy to control, especially in the field. As a result, several commercial products containing living organisms, including bacteria such as P. fluorescens have not been effective (Nicot et al., 2012, Kohl et al., 2019). It has been argued that products derived from microorganisms (biopesticides) may be more effective that living microorganisms because they are easier to formulate and control (Glare et al., 2012). In our case, the potential use of the secreted fraction of P. protegens as a biopesticide would be even more desirable, because it is likely that this preparation contains several bioactive molecules with diverse modes of action. Using a preparation with multiple modes of action can reduce the probability that bacteria develop resistance, which had previously hampered efforts to control bacterial diseases in plants (Sundin & Wang, 2018).

### REFERENCES

Bankevich A, Nurk S, Antipov D, et al., 2012. SPAdes: a new genome assembly algorithm and its applications to single-cell sequencing. J. Comput. Biol. 19, 455-77.
Bao E, Jiang T, Girke T, 2014. AlignGraph: algorithm for secondary de novo genome assembly guided by closely related references. Bioinformatics 30, 1319-128.
Baym M, Kryazhimskiy S, Lieberman TD, Chung H, Desai MM, Kishony R, 2015. Inexpensive multiplexed library preparation for megabase-sized genomes. PLOS One 10, e0128036.
Bertani G, 1951. Studies on lysogenesis. I. The mode of phage liberation by lysogenic Escherichia coli. J Bacteriol 62, 293-300.
Blin, K., Shaw, S., Steinke, K., Villebro, R., Ziemert, N., Lee, S.Y., Medema, M.H., and Weber, T. 2019. antiSMASH 5.0: updates to the secondary metabolite genome mining pipeline. Nucleic acids research 47: W81-W87.
Boetzer M, Henkel CV, Jansen HJ, Butler D, Pirovano W, 2011. Scaffolding pre-assembled contigs using SSPACE. Bioinformatics 27, 578-9.
Bolger AM, Lohse M, Usadel B, 2014. Trimmomatic: a flexible trimmer for Illumina sequence data. Bioinformatics 30, 2114-20.
Chin AWTF, Bloemberg GV, Mulders IH, Dekkers LC, Lugtenberg BJ, 2000. Root colonization by phenazine-1-carboxamide-producing bacterium Pseudomonas chlororaphis PCL1391 is essential for biocontrol of tomato foot and root rot. Mol Plant Microbe Interact 13, 1340-5.
Cole JR, Wang Q, Fish JA, et al., 2014. Ribosomal Database Project: data and tools for high throughput rRNA analysis. Nucleic Acids Res 42, D633-42.
D'aes J, Hua GK, De Maeyer K, et al., 2011. Biological control of Rhizoctonia root rot on bean by phenazine- and cyclic lipopeptide-producing Pseudomonas CMR12a. Phytopathology 101, 996-1004.
Degrassi G, Devescovi G, Kim J, Hwang I, Venturi V, 2008. Identification, characterization and regulation of two secreted polygalacturonases of the emerging rice pathogen Burkholderia glumae. FEMS Microbiol Ecol 65, 251-62.
Devescovi G, Bigirimana J, Degrassi G, et al., 2007. Involvement of a Quorum-Sensing-Regulated Lipase Secreted by a Clinical Isolate of Burkholderia glumae in Severe Disease Symptoms in Rice. Appl Environ Microbiol 73, 4950-8.
Dubouzet JG, Maeda S, Sugano S, et al., 2011. Screening for resistance against Pseudomonas syringae in rice-FOX Arabidopsis lines identified a putative receptor-like cytoplasmic kinase gene that confers resistance to major bacterial and fungal pathogens in Arabidopsis and rice. Plant Biotechnol J 9, 466-85.
Fory PA, Triplett L, Ballen C, et al., 2014. Comparative analysis of two emerging rice seed bacterial pathogens. Phytopathology 104, 436-44.
Glare T, Caradus J, Gelernter W, et al., 2012. Have biopesticides come of age? Trends Biotechnol 30, 250-8.
Gross, H., Stockwell, V.O., Henkels, M.D., Nowak-Thompson, B., Loper, J.E., and Gerwick, W.H. 2007. The genomisotopic approach: a systematic method to isolate products of orphan biosynthetic gene clusters. Chem Biol 14:53-63.
Gutierrez-Barranquero JA, De Vicente A, Carrion VJ, Sundin GW, Cazorla FM, 2013. Recruitment and rearrangement of three different genetic determinants into a conjugative plasmid increase copper resistance in Pseudomonas syringae. Appl Environ Microbiol 79, 1028-33.
Haas D, Defago G, 2005. Biological control of soil-borne pathogens by fluorescent pseudomonads. Nat Rev Microbiol 3, 307-19.
Ham JH, Melanson RA, Rush MC, 2011. Burkholderia glumae: next major pathogen of rice? Molecular Plant Pathology 12, 329-39.
Hanahan D, 1983. Studies on transformation of Escherichia coli with plasmids. Journal of Molecular Biology 166, 557-80.
Hikichi Y, 1993. Antibacterial activity of oxolinic acid on Pseudomonas glumae. Japanese Journal Phytopath 59, 369-74.
Howell CR, Stipanovic RD, 1979. Control of Rhizoctonia solani on cotton seedlings with Pseudomonas fluorescens and with an antibiotic produced by the bacterium. Phytopathology 69, 480-2.
Howell CR, Stipanovic RD, 1980. Suppression of Pythium ultimum-induced damping-off of cotton seedlings by Pseudomonas fluorescens and its antibiotic, pyoluteorin. Phytopathology 70, 712-15.
Iiyama K, Furuya N, Takanami Y, Noraki M, 1995. A role of phytotoxin in virulence of Pseudomonas glumae Kurita et Tabeti. Japanese Journal Phytopath 61, 470-6.
Jamali F, Sharifi-Tehrani A, Lutz MP, Maurhofer M, 2009. Influence of host plant genotype, presence of a pathogen, and coinoculation with Pseudomonas fluorescens strains on the rhizosphere expression of hydrogen cyanide- and 2,4-diacetylphloroglucinol biosynthetic genes in P. fluorescens biocontrol strain CHA0. Microb Ecol 57, 267-75.
Jolley KA, Bliss CM, Bennett JS, et al., 2012. Ribosomal multilocus sequence typing: universal characterization of bacteria from domain to strain. Microbiology (Reading, England) 158, 1005-15.
Kang Y, Kim J, Kim S, et al., 2008. Proteomic analysis of the proteins regulated by HrpB from the plant pathogenic bacterium Burkholderia glumae. Proteomics 8, 106-21.
Karki HS, Shrestha BK, Han JW, et al., 2012b. Diversities in virulence, antifungal activity, pigmentation and DNA fingerprint among strains of Burkholderia glumae. PLOS One 7, e45376.
Kawaradani M, Okada K, Kusakari SI, 2000. New selective medium for isolation of Burkholderia glumae from rice seeds. J. Gen. Plant Pathol 66, 234-7.
Keel C, Schnider U, Maurhofer M, et al., 1992. Suppression of root diseases by Pseudomonas fluorescens CHAO: importance of the bacterial secondary metabolite 2,4-diaceylphloroglucinol. Molecular Plant-Microbe Interactions 5, 4-13.
Kim J, Kim JG, Kang Y, et al., 2004. Quorum sensing and the LysR-type transcriptional activator ToxR regulate toxoflavin biosynthesis and transport in Burkholderia glumae. Mol Microbiol 54, 921-34.
King EO, Ward MK, Raney DE, 1954. Two simple media for the demonstration of pyocyanin and fluorescin. Journal of Laboratory and Clinical Medicine 44, 301-7.
Kloepper JW, Leong J, Teintze M, Schroth MN, 1980. Pseudomonas siderophores: a mechanism explaining disease-suppressive soils. Current microbiology 4, 317-20.
Knapp A, Voget S, Gao R, et al., 2016. Mutations improving production and secretion of extracellular lipase by Burkholderia glumae PG1. Appl Microbiol Biotechnol 100, 1265-73.
Kohl J, Kolnaar R, Ravensberg WJ, 2019. Mode of Action of Microbial Biological Control Agents Against Plant Diseases: Relevance Beyond Efficacy. Front Plant Sci 10, 845.
Lee YH, Ko SJ, Cha KH, Park EW, 2015. BGRcast: A Disease Forecast Model to Support Decision-making for Chemical Sprays to Control Bacterial Grain Rot of Rice. Plant Pathol J 31, 350-62.
Lane, D. J. 1991. 16S/23S rRNA sequencing, p. 115-175. In E. Stackebrandt and M. Goodfellow (ed.), Nucleic acid techniques in bacterial systematics. John Wiley and Sons, Chichester, United Kingdom.
Lemanceau P, Bakker PA, De Kogel WJ, Alabouvette C, Schippers B, 1992. Effect of pseudobactin 358 production by Pseudomonas putida WCS358 on suppression of Fusarium wilt of carnations by nonpathogenic Fusarium oxysporum Fo47. Appl Environ Microbiol 58, 2978-82.
Lim J, Lee TH, Nahm BH, Do Choi Y, Kim M, Hwang I, 2009. Complete Genome Sequence of Burkholderia glumae BGR1. Journal of Bacteriology 191, 3758-9.
Maeda S, Hayashi N, Sasaya T, Mori M, 2016. Overexpression of BSR1 confers broad-spectrum resistance against two bacterial diseases and two major fungal diseases in rice. Breed Sci 66, 396-406.
Maeda Y, Kiba A, Ohnishi K, Hikichi Y, 2004. Implications of amino acid substitutions in GyrA at position 83 in terms of oxolinic acid resistance in field isolates of Burkholderia glumae, a causal agent of bacterial seedling rot and grain rot of rice. Appl Environ Microbiol 70, 5613-20.
Magoč T, Salzberg SL, 2011. FLASH: fast length adjustment of short reads to improve genome assemblies. Bioinformatics 27, 2957-63.
Manulis S, Kleitman F, Shtienberg D, et al., 2003. Changes in the Sensitivity of Erwinia amylovora Populations to Streptomycin and Oxolinic Acid in Israel. Plant Dis 87, 650-4.
Maurhofer M, Keel C, Defago G, 1994. Pyoluteorin production by Pseudomonas fluorescens strain CHA0 is involved in the suppression of Phythium damping-off of cress but not of cucumber. Eur J Plant Pathol 15, 221-32.
Mazurier S, Corberand T, Lemanceau P, Raaijmakers JM, 2009. Phenazine antibiotics produced by fluorescent pseudomonads contribute to natural soil suppressiveness to Fusarium wilt. ISME J 3, 977-91.
Milus EA, Rothrock CS, 1997. Efficacy of Bacterial Seed Treatments for Controlling Pythium Root Rot of Winter Wheat. Plant Dis 81, 180-4.
Mizobuchi R, Sato H, Fukuoka S, et al., 2013. Mapping a quantitative trait locus for resistance to bacterial grain rot in rice. Rice (N Y) 6, 13.
Mizobuchi R, Fukuoka S, Tsushima S, Yano M, Sato H, 2016. QTLs for Resistance to Major Rice Diseases Exacerbated by Global Warming: Brown Spot, Bacterial Seedling Rot, and Bacterial Grain Rot. Rice (N Y) 9, 23.
Mizobuchi R, Fukuoka S, Tsuiki C, Tsushima S, Sato H, 2018. Evaluation of major Japanese rice cultivars for resistance to bacterial grain rot caused by Burkholderia glumae and identification of standard cultivars for resistance. Breed Sci 68, 413-9.
Mondal KK, Mani C, Verma G, 2015. Emergence of Bacterial Panicle Blight caused by Burkholderia glumae in North India. Plant Disease 99, 1268.
Mulaw T, Wamishe Y, Jia Y, 2018. Characterization and in Plant Detection of Bacteria that Cause Bacterial Panicle Blight of Rice. Research Journal of Plant Pathology 1, 1-7.
Nandakumar R, Rush MC, Correa F, 2007. Association of Burkholderia glumae and B. gladioili with Panicle Blight Symptoms on Rice in Panama. Plant Dis 91, 767.
Nandakumar R, Shahjahan AKM, Yuan XL, et al., 2009. Burkholderia glumae and B. gladioli cause bacterial panicle blight in rice in the Southern United States. Plant Dis 93, 896-905.
Nicot PC, Alabouvette C, Bardin M, Blum B, Kohl J, Ruocco M, 2012. Review of factors influencing the success or failure of biocontrol: technical, industrial and socio-economic perspectives. In. IOBC-WPRS Bulletin. 78, 95-98.
Olorunleke, F.E., Hua, G.K., Kieu, N.P., Ma, Z., and Hofte, M. 2015. Interplay between orfamides, sessilins and phenazines in the control of Rhizoctonia diseases by Pseudomonas sp. CMR12a. Environ Microbiol Rep 7:774-781.
Pieterse CM, Zamioudis C, Berendsen RL, Weller DM, Van Wees SC, Bakker PA, 2014. Induced systemic resistance by beneficial microbes. Annu Rev Phytopathol 52, 347-75.
Rasband, W.S., ImageJ, U.S. National Institutes of Health, Bethesda, Maryland, USA, https://imagej.nih.gov/ij/, 1997-2018.
Raaijmakers JM, Mazzola M, 2012. Diversity and natural functions of antibiotics produced by beneficial and plant pathogenic bacteria. Annu Rev Phytopathol 50, 403-24.
Rodriguez C, Lang L, Wang A, Altendorf K, Garcia F, Lipski A, 2006. Lettuce for human consumption collected in Costa Rica contains complex communities of culturable oxytetracycline- and gentamicin-resistant bacteria. Appl Environ Microbiol 72, 5870-6.
Seemann T, 2014. Prokka: rapid prokaryotic genome annotation. Bioinformatics 30, 2068-9.
Shew AM, Durand-Morat A, Nalley LL, Zhou XG, Rojas C, Thoma G, 2019. Warming increases Bacterial Panicle Blight (Burkholderia glumae) occurrences and impacts on USA rice production. PLOS One 14, e0219199.
Shrestha BK, Karki HS, Groth DE, Jungkhun N, Ham JH, 2016. Biological Control Activities of Rice-Associated Bacillus sp. Strains against Sheath Blight and Bacterial Panicle Blight of Rice. PLOS One 11, e0146764.
Song L, Florea L, Langmead B, 2014. Lighter: fast and memory-efficient sequencing error correction without counting. Genome Biol. 15, 509.
Suarez-Moreno ZR, Vinchira-Villarraga DM, Vergara-Morales DI, et al., 2019. Plant-Growth Promotion and Biocontrol Properties of Three Streptomyces spp. Isolates to Control Bacterial Rice Pathogens. Front Microbiol 10, 290.
Sundin GW, Bender CL, 1993. Ecological and genetic analysis of copper and streptomycin resistance in Pseudomonas syringae pv. syringae. Appl Environ Microbiol 59, 1018-24.
Sundin GW, Castiblanco LF, Yuan X, Zeng Q, Yang CH, 2016. Bacterial disease management: challenges, experience, innovation and future prospects: Challenges in Bacterial Molecular Plant Pathology. Mol Plant Pathol 17, 1506-18.
Sundin GW, Wang N, 2018. Antibiotic Resistance in Plant-Pathogenic Bacteria. Annu Rev Phytopathol 56, 161-80.
Temesgen M, Wamishe Y, Jia Y, 2018. Characterization and plant detection of bacteria that cause Bacterial Panicle Blight of rice. American Journal of Plant Sciences 9, 667-84.
Thomashow LS, Bonsall RE, Weller DM, 1997. Antibiotic production by soil and rhizosphere microbes in situ. In: Hurst CJ, Knudsen GR, Mcinerney MJ, Stetzenbach LD, Walter MV, eds. Manual of Environmental Microbiology, ed. Washington, DC: ASM Press, 493-99.
Thomashow LS, Weller DM, 1988. Role of a phenazine antibiotic from Pseudomonas fluorescens in biological control of Gaeumannomyces graminis var. tritici. J Bacteriol 170, 3499-508.
Voisard C, Keel C, Haas D, Defago G, 1989. Cyanide production by Pseudomonas fluorescens helps suppress black root rot of tobacco under gnotobiotic conditions. EMBO J 8, 351-8.
Walker BJ, Abeel T, Shea T, et al., 2014. Pilon: an integrated tool for comprehensive microbial variant detection and genome assembly improvement. PLOS One 9, e112963.
Wamishe Y, Kelsey C, Belmar SB, Gerbremariam T, Mccarty D, 2015. Bacterial Panicle Blight of Rice in Arkansas. University of Arkansas Division of Agriculture Research and Extension: Agriculture and Natural Resources.
Weller DM, 2007. Pseudomonas biocontrol agents of soilborne pathogens: looking back over 30 years. Phytopathology 97, 250-6.
Yasmin S, Zaka A, Imran A, et al., 2016. Plant Growth Promotion and Suppression of Bacterial Leaf Blight in Rice by Inoculated Bacteria. PLOS One 11, e0160688.
Zhou XG, 2014. First Report of Bacterial Panicle Blight of Rice Caused by Burkholderia glumae in South Africa. Plant Dis 98, 566.

### DEPOSIT INFORMATION

A deposit of the Board of Trustees of the University of Arkansas proprietary Pseudomonas protegens strain designated as PBL3 disclosed above and recited in the appended claims has been made with the ARS Culture Collection (NRRL), 1815 N. University Street, Peoria, IL 61604, and has been accepted under the terms of the Budapest Treaty. The date of deposit was Nov. 17, 2021. The deposit comprises 5 liquid nitrogen stocks of the strain, which were found viable on Nov. 18, 2021. All restrictions will be irrevocably removed upon granting of a patent, and the deposit is intended to meet all of the requirements of 37 C.F.R. §§ 1.801-1.809. The NRRL Accession Number is B-68083. The deposit will be maintained in the depository for a period of thirty years, or five years after the last request, or for the enforceable life of the patent, whichever is longer, and will be replaced as necessary during that period.

1.-5. (canceled)

6. A method of inhibiting the growth of a microorganism, the method comprising: contacting the microorganism with the antimicrobial composition of claim 21, thereby inhibiting its growth.

7. The method of claim 6, wherein the growth of the microorganism is inhibited on a plant.

8. The method of claim 7, wherein the plant is a rice plant.

9. The method of claim 7, wherein the contacting is carried out by spraying or dusting the plant or a portion of the plant with the antimicrobial composition.

10. The method of claim 7, wherein the contacting is carried out before flowering or during panicle formation.

11. The method of claim 6, wherein the microorganism is a bacterium or a fungus.

12. The method of claim 11, wherein the microorganism is Burkholderia glumae.

13.-19. (canceled)

20. The method of claim 12, wherein the growth of the microorganism is inhibited on a plant, and wherein the plant is a rice plant and the method reduces symptoms of bacterial panicle blight.

21. An antimicrobial composition comprising pyoverdin, 2,4-diacetylphloroglucinol (2,4-DAPG), pyochelin, pyoluteorin, or a combination thereof.

22. The antimicrobial composition of claim 21, wherein the composition comprises two or more of pyoverdin, 2,4-DAPG, pyochelin, and pyoluteorin.

23. The antimicrobial composition of claim 22, wherein the composition comprises three or more of pyoverdin, 2,4-DAPG, pyochelin, and pyoluteorin.

24. The antimicrobial composition of claim 23, wherein the composition comprises pyoverdin, 2,4-DAPG, pyochelin, and pyoluteorin.

25. The antimicrobial composition of claim 21, wherein the composition is an agricultural composition.
